(19) United States
(12) Patent Application Publication
(10) Pub. No.: US 2020/0392005 A1
(43) Pub. Date: Dec. 17, 2020

(54) PARTICULATE MATERIAL PRODUCTION PROCESS

(71) Applicant: N4 Pharma UK Limited, Derby, Derbyshire (GB)

(72) Inventors: Chengzhong (Michael) Yu, Brisbane (AU); Hao Song, Brisbane (AU); Graham Worrall, Wilton (GB); Lynn Donlon, Wilton (GB)

(73) Assignee: N4 Pharma UK Limited, Derby, Derbyshire (GB)

(21) Appl. No.: 16/764,267

(22) PCT Filed: Nov. 14, 2018
(86) PCT No.: PCT/GB2018/053298

§ 371 Date: May 14, 2020

(30) Foreign Application Priority Data
Nov. 14, 2017 (GB) 1718817.8

(51) Int. Cl. C01B 33/18; B01J 13/04; B01J 13/20; C12N 15/88; B29B 9/12

(52) U.S. Cl. CPC C01B 33/18; B01J 13/04; B01J 13/203; C12N 15/88; B29B 2009/163; C01P 2004/64; C01P 2004/34; C01P 2004/03; C01P 2002/88; B29B 9/12

(57) ABSTRACT

The present invention relates to a process for producing a plurality of hollow inorganic nanoparticles, which process comprises: (a) contacting a first monomer and a second monomer in a solvent to produce a composition comprising the solvent and a plurality of polymer nanoparticles; (b) adding an inorganic compound precursor to the composition comprising the solvent and the plurality of polymer nanoparticles to produce a composition comprising the solvent and a plurality of inorganic compound-coated polymer nanoparticles; (c) adding an additional amount of the first and second monomers to the composition comprising the solvent and the plurality of inorganic compound-coated polymer nanoparticles to produce a composition comprising the solvent and a plurality of composite nanoparticles; and (d) heating the plurality of composite nanoparticles to produce the plurality of hollow inorganic nanoparticles, wherein in step (a) the first monomer and the second monomer are contacted in the solvent at a temperature of at least 30° C. The present invention also relates to plurality of hollow inorganic nanoparticles and uses thereof.

### FIELD OF THE INVENTION

The present invention relates to a process for producing a plurality of hollow inorganic nanoparticles. The invention also relates to a plurality of hollow inorganic nanoparticles, compositions comprising the nanoparticles and uses of those compositions.

### BACKGROUND OF THE INVENTION

Hollow nanoparticles comprising inorganic materials have been found to have a wide range of applications. WO 2015/089590 A1 describes silica vesicles and their use as vehicles for delivery of active agents.

A process for producing rough mesoporous hollow silica nanoparticles is described in WO 2016/164987 A1. The process proceeds via the initial formation of polymer nanoparticles which are subsequently coated with silica before the introduction of further polymer. The process of WO 2016/164987 A1 involves a lengthy synthetic process followed by calcination.

It is desirable to provide a more efficient process for the production of hollow inorganic nanoparticles. It is also desirable to provide a process which produces nanoparticles having improved morphology and/or particle size distribution.

### SUMMARY OF THE INVENTION

The inventors have surprisingly found that the efficiency of a process for producing a plurality of hollow inorganic nanoparticles may be significantly improved by increasing the temperature at which initial formation of the polymer nanoparticles is carried out. This change can allow for a dramatic reduction in the time taken to produce the hollow inorganic nanoparticles and has been found not to negatively affect the morphology of the nanoparticles. It has also been surprisingly found that the improved process can lead to the production of nanoparticles having improved surface morphology. An increase in the monodispersity of the hollow inorganic nanoparticles may also be observed. The hollow inorganic nanoparticles according to the invention have also been found to have an adjuvant effect when used in therapy.

The invention provides a process for producing a plurality of hollow inorganic nanoparticles, which process comprises: (a) contacting a first monomer and a second monomer in a solvent to produce a composition comprising the solvent and a plurality of polymer nanoparticles; (b) adding an inorganic compound precursor to the composition comprising the solvent and the plurality of polymer nanoparticles to produce a composition comprising the solvent and a plurality of inorganic compound-coated polymer nanoparticles; (c) adding an additional amount of the first and second monomers to the composition comprising the solvent and the plurality of inorganic compound-coated polymer nanoparticles to produce a composition comprising the solvent and a plurality of composite nanoparticles; and (d) heating the plurality of composite nanoparticles to produce the plurality of hollow inorganic nanoparticles, wherein in step (a) the first monomer and the second monomer are contacted in the solvent at a temperature of at least 30° C.

The invention also provides a plurality of hollow inorganic nanoparticles obtainable by a process according to the invention.

Further provided by the invention is a plurality of hollow inorganic nanoparticles, wherein each of the hollow inorganic nanoparticles comprises: a shell comprising an inorganic compound; a volume within the shell which does not comprise the inorganic compound; and disposed on the exterior of the shell, a plurality of protrusions comprising the inorganic compound. The particle size of the plurality of hollow inorganic nanoparticles is typically from 100 to 500 nm. The hollow inorganic nanoparticles may further comprise a plurality of acidic groups bound to the inorganic compound.

The invention further provides a composition comprising a plurality of hollow inorganic nanoparticles according to the invention and an active agent.

Also provided by the invention is a composition according to the inventio or use in the treatment of the human or animal body by therapy.

Also provided by the invention is a plurality of hollow inorganic nanoparticles according to the invention for use as an adjuvant in the treatment of the human or animal body by therapy.

The invention also provides a method for controlling pests at a locus, which method comprises exposing the locus to a composition according to the invention.

### BRIEF DESCRIPTION OF THE FIGURES

FIG. 1: SEM images of SiNP produced during Synthesis SiNP001. Upper images: coated particles, Lower images: uncoated particles.

FIG. 2: SEM images of SiNP produced during synthesis SiNP002. Upper images: coated particles, Lower images: uncoated particles.

FIG. 3: On-line monitoring of reaction temperature, pH and stirrerspeed showing consistency throughout the synthesis.

FIG. 4: Evolution of SiNP particle size measured using dynamic light scattering.

FIG. 5: SEM images of uncoated SiNP produced during synthesis SiNP003.

FIG. 6: SEM images of uncoated SiNP produced during synthesis SiNP004.

FIG. 7: TGA analysis of the calcination process for SiNP produced during synthesis SiNP004.

FIG. 8: SEM images of uncoated SiNP produced during synthesis SiNP004, 14 hour calcination regime.

FIG. 9: SEM images of SiNP prepared during synthesis SiNP005. Upper images and lower right image: uncoated particles; lower left image: coated particles.

FIG. 10: SEM images of uncoated SiNP prepared during synthesis SiNP005 V2.

FIG. 11: SEM images of uncoated SiNP prepared during synthesis SiNP006.

FIG. 12: SEM ages of uncoated SiNP prepared during synthesis SiNP006

FIG. 13: SEM images of uncoated SiNP prepared during synthesis SiNP006 III FIG. 14: SEM images of uncoated SiNP prepared during synthesis SiNP006 IV

FIG. 15: SEM images of uncoated SiNP prepared during synthesis SiNP007 in which the initial monomer concentration was reduced by 25%. Note particle size has been reduced and morphology retained.

FIG. 16: SEM images of uncoated SiNP prepared during synthesis SiNP007 II in which the initial monomer concentration was reduced by 25% and cool down time increased by 30 minutes. Note particle size has increased however desired morphology is retained.

FIG. 17: SEM images of uncoated SiNP prepared during synthesis SiNP007 V in which the initial monomer concentration was reduced by 25%. Note correct particle size and morphology.

FIG. 18: TEM images of SiNPs.

FIG. 19: SEM images of uncoated SiNP prepared during synthesis SiNP008. Note monomodal dispersion of particles, correct particle size and ‘spiky’ morphology.

FIG. 20: SEM images of uncoated SiNP prepared during synthesis SiNP008. Note monomodal dispersion of particles, correct particle size and ‘spiky’ morphology.

FIG. 21: SEM images of uncoated SiNP prepared in SiNP0008 calcined using different ramp rates. Note monomodal dispersion of particles and correct particle size. Morphology appears less ‘spiky’ than using the standard 2° C./min ramp rate during calcination and some agglomeration is also observed.

FIG. 22: Thermogravimetric analysis of calcination process at different ramp rates for SiNP produced during synthesis of SiNP0008 II.

FIG. 23: SEM images of uncoated SiNP prepared in SiNP0009. Particle size and morphology appear to be correct, however significant agglomeration is observed.

FIG. 24: SEM images of uncoated SiNP prepared in SiNP0009 II. Particles show the desired ‘spiky’ morphology however note large particle size and agglomerations.

FIG. 25: SEM images of uncoated resorcinol formaldehyde particles prepared in SiNP0009 III. Note large particle size and agglomerations.

FIG. 26: SEM images of uncoated resorcinol formaldehyde particles prepared in SiNP0009 III. Note large particle size and agglomerations.

FIG. 27: SEM images of uncoated SiNP prepared in SiNP0010. Note that holes are observed in the walls of some of the particles.

FIG. 28: SEM images of uncoated SiNP prepared in SiNP0011. Note monomodal dispersion of particles, correct particle size and ‘spiky’ morphology

FIG. 29: SEM images of uncoated SiNP prepared in SiNP0011. Note monomodal dispersion of particles, correct particle size and ‘spiky’ morphology

FIG. 30: TEM images of SiNPs.

FIG. 31: SEM images of uncoated resorcinol formaldehyde particles prepared in SiNP0012. Note large particle sizes with particle distribution is monomodal.

FIG. 32: SEM images of uncoated resorcinol formaldehyde particles prepared in SiNP0012 II. Note large particle size.

FIG. 33: SEM images of uncoated resorcinol formaldehyde particles prepared in SiNP0012 III. Note large particle size.

FIG. 34: SEM images of uncoated resorcinol formaldehyde particles prepared in SiNP0012 IV.

FIG. 35: Evolution of the zeta potential on SNP008 coated and uncoated as a function of pH.

FIG. 36: Evolution of the zeta potential on PEI loaded SNP008 with different conditions as a function of pH.

FIG. 37: Evolution of zeta potential on phosphonate linked SNP008 as a function of pH.

FIG. 38: Evolution of carbon content during the phosphonate linking step.

FIG. 39: Evolution of the zeta potential on SNP008 at different times during the PEI loading as a function of pH.

FIG. 40: Evolution of EP as a function of time during PEI loading.

FIG. 41: Evolution of zeta potential on SNP011 as a function of pH after 30 min of PEI Loading.

FIG. 42: Evolution of zeta potential on SNP011 II as a function of pH after 5 min of PEI loading.

FIG. 43: Evolution of N content during PEI loading for two different particles treated in the same way.

FIG. 44: SEM image of SiNP NUMed silica nanoparticles.

FIG. 45: TEM image of SiNP NUMed silica nanoparticles.

FIG. 46: Effect of ovalbumin (OVA) DNA on splenocyte proliferation when administered using different vehicles.

FIG. 47: Transfection efficiency of SiNPs loaded with pDNA encoding luciferase.

FIG. 48: (a) Schematic illustration of synthesis of silica nanoparticles with smooth, raspberry and rambutan like surface topology, (b) TEM images of S-SNPs, (c) Ras-SNPs and (d) Ram-SNPs, (e) nitrogen sorption isotherms and (f) corresponding pore size distribution of these nanoparticles and (g) zeta potential of silica nanoparticles before and after PEI conjugation.

FIG. 49: PEI conjugation mode on silica nanoparticles: covalent binding using 3-GPS and strong electrostatic attraction using THPMP.

FIG. 50: Plasmid DNA loading capacity of silica nanoparticles covalently modified with PEI of different molecular weight.

FIG. 51: Fluorescent microscopy and flow cytometry analysis of eGFP-pcDNA transfection efficiency in HEK-293T cells using Ram-SNPs modified with 10 k PEI via different approaches.

### DETAILED DESCRIPTION OF THE INVENTION

The invention provides a process for producing a plurality of hollow inorganic nanoparticles, which process comprises: (a) contacting a first monomer and a second monomer in a solvent to produce a composition comprising the solvent and a plurality of polymer nanoparticles; (b) adding an inorganic compound precursor to the composition comprising the solvent and the plurality of polymer nanoparticles to produce a composition comprising the solvent and a plurality of inorganic compound-coated polymer nanoparticles; (c) adding an additional amount of the first and second monomers to the composition comprising the solvent and the plurality of inorganic compound-coated polymer nanoparticles to produce a composition comprising the solvent and a plurality of composite nanoparticles; and (d) heating the plurality of composite nanoparticles to produce the plurality of hollow inorganic nanoparticles, wherein in step (a) the first monomer and the second monomer are contacted in the solvent at a temperature of at least 30° C.

Contacting the first monomer and the second monomer typically comprises allowing the first and second monomers to react. For instance, the first and second monomers may both be dissolved in the solvent.

The process of the invention involves forming the plurality of polymer nanoparticles at a temperature above room temperature. The entirety of step (a) is typically carried out at a temperature of at least 30° C. The first monomer and the second monomer are typically contacted in the solvent at a temperature of from 30° C. to 70° C. Preferably, the first and second monomers may be contacted in the solvent at a temperature of from 40.0° C. to 50.0° C. For instance the temperature may be from 42.0° C. to 48.0° C. or the temperature may be about 45° C.

The first and second monomers are contacted at a temperature of at least 30° C. for typically no more than four hours (i.e. no more than 240 minutes) prior to addition of the inorganic compound precursor.

Typically, the first and second monomers are contacted for from 10 minutes to 180 minutes, for instance from 30 minutes to 150 minutes. The first and second monomers may be contacted for from 60 minutes to 120 minutes, for example from 80 minutes to 100 minutes. When the first and second monomers are contacted in the solvent for a specific amount of time, typically either: step (b) is initiated after that specific amount of time; the reaction is temperature is reduced after that specific amount of time; or the reaction is quenched after the specific amount of time (for instance by adding an additional amount of the solvent).

For instance, the first and second monomers may be contacted at a temperature of from 40.0° C. to 50.0° C. for from 30 minutes to 150 minutes before cooling the composition comprising the solvent and the first and second monomers to a temperature of less than 30° C.

The hollow inorganic nanoparticles are nanoparticles which are hollow (i.e, which comprise a shell comprising a material around a central volume which does not comprise the material) and which comprise an inorganic compound (which may also be referred to as an inorganic material). The inorganic compound may be any suitable inorganic compound. For instance, the inorganic compound may be an oxide. The inorganic compound is typically silica (i.e. SiO₂), titania (TiO₂) or alumina (Al₂O₃). The inorganic compound is preferably silica and the hollow inorganic nanoparticles are preferably hollow silica nanoparticles. The term “silica” should be understood to include oxides of silicon, typically silicon dioxide.

The hollow inorganic nanoparticles typically comprise at least 70% by weight of the inorganic compound relative to the total weight of the hollow inorganic nanoparticles. For instance, the hollow inorganic nanoparticles may comprise at least 90% by weight of the inorganic compound or at least 95% by weight of the inorganic compound. The plurality of hollow inorganic nanoparticles may consist of, or consist essentially of, the inorganic compounds. These weight percentages are prior to the loading of the plurality of hollow inorganic nanoparticles with an active agent.

A composition which consists essentially of a specified component comprises the specified component and any other component in an amount (for instance less than 0.5 wt %) which does not materially affect the function of the specified component.

Step (a) comprises contacting the first and second monomers in the solvent, for instance by mixing the first monomer and the second monomer in the solvent. The solvent may be any suitable solvent, for instance a solvent suitable for carrying out the Stöber process (Stöber et al, Journal of Colloid and Interface Science. 26 (1): 62-69; 1968). The solvent may comprise a polar solvent. The polar solvent may be a polar protic solvent such as water, an alcohol or a carboxylic acid, or a polar aprotic solvent such as a ketone (for instance acetone), a nitrile (for instance acetonitrile), a haloalkane (for instance chloromethane or dichloromethane) or a haloarene (for instance chlorobenzene). Typically the solvent comprises water and/or an alcohol, which alcohol may be methanol, ethanol, n-propanol or isopropanol. Typically the solvent comprises ethanol and water. The volume ratio ethanol:water is typically from 60:20 to 80:5, for instance about 70:10.

The solvent may typically comprise a base (i.e. the solvent may be a composition comprising inert liquids which act as a solvent and a base which acts as a catalyst). The base is typically a compound comprising nitrogen, for instance ammonia, ammonium hydroxide or an alkyl amine. The solvent typically comprises ammonia or ammonium hydroxide. The solvent may comprise from 0.0 to 10.0 vol % of 28-30 vol % ammonia solution.

The solvent preferably comprises water, an alcohol and ammonia. The pH of the solvent is typically at least 9.0, for instance from 10.0 to 12.0.

The reaction of the first and second monomers to form the plurality of polymer nanoparticles typically comprises stirring the composition comprising the first and second monomers and the solvent. The composition may be stirred at a rate of from 50 to 500 rpm, for instance from 200 to 400 rpm.

The first and second monomers may be any monomers suitable for forming the plurality or polymer nanoparticles. The first monomer is typically a compound comprising one or more hydroxyl groups and the second monomer is typically a compound comprising one or more aldehyde groups. More typically, the first monomer is a diol and the second compound is an aldehyde. Examples of diols include ethane-1,2-diol, propane-1,3-diol and benzenediol. For instance, the first monomer may be a compound of formula HO—Ar—OH and the second monomer may be a compound of formula HC(O)—R¹, where Ar is an substituted or unsubstituted aryl group and R¹ is H or substituted or unsubstituted C₁₋₆ alkyl.

A substituted group may comprise one or more substituents selected from C₁₋₆ alkyl, hydroxyl, oxo, halo, amino, nitro or carboxylate.

An C₁₋₆ alkyl group is a saturated hydrocarbon radical containing a linear or branched chain of from 1 to 6 carbon atoms. C₁₋₆ alkyl may be methyl, ethyl, n-propyl, iso-propyl, n-butyl, sec-butyl, tert-butyl, pentyl, neo-pentyl and hexyl. Typically R¹ is H or methyl. The first monomer may for instance be formaldehyde or ethanal.

Ar may be a substituted or unsubstituted phenyl group. For instance, Ar may be phenyl, methylphenyl, dimethylphenyl or chlorophenyl. The second monomer may be benzene diol, for instance resorcinol, catechol or hydroquinone.

Preferably the first onomer is resorcinol and the second monomer is formaldehyde.

The first onomer may alternatively be a C₁₋₆ alkylamine, for instance methylamine.

The concentration of the first monomer is typically from 1.0 mM to 0.1 M and the concentration of the second monomer is typically from 1.0 mM to 0.1 M. For instance, the concentration of the first monomer may be from 0.01 M to 0.03 M and the concentration of the second monomer may be from 0.3 to 0.05 M.

The concentration of the first monomer (for instance resorcinol) in the solvent may for instance be from 1.0 mg/ml to 3.0 mg/ml or from 1.2 mg/ml to 2.0 mg/ml. For instance, from 0.1 to 0.4 g of resorcinol may be added for each 80 ml of solvent.

The concentration of the second monomer (for instance formaldehyde) in the solvent may for instance be from 0.001 to 0.005 ml of a solution comprising from 20 to 50 wt % of the second monomer/ml of solvent. For instance, from 0.1 to 0.4 ml of 37 wt % aqueous solution of formaldehyde may be added for each 80 ml of solvent.

The molar ratio (first monomer):(second monomer) is typically from 3.0:1.0 to 1.0:3.0 or from 2.0:1.0 to 1.0:2.0. There may for instance be a molar excess of the first monomer (e.g. resorcinol) and the molar ratio (first monomer):(second monomer) may be from 2.0:1.0 to 1.1:1.0.

Contacting of the first and second monomer produces a plurality of polymer nanoparticles, i.e. a plurality of nanoparticles comprising the polymer resulting from reaction of the first and second monomers. The polymer is typically a co-polymer of the first and second monomers. The polymer is typically a condensation polymer. For instance, the polymer may be a polyether, a polyester or a polyamide. The polymer is typically a cross-linked polymer (e.g. as opposed to a linear polymer).

Preferably the polymer comprises a resorcinol-formaldehyde co-polymer.

The average particle size (e.g. mean particle size) of the plurality of polymer nanoparticles is typically from 50 to 500 nm, for instance from 100 to 300 nm.

References to average particle size herein are typically references to average particle size as measured from a particle size distribution determined using dynamic light scattering. The dynamic light scattering may for instance be measured using a Horiba SZ-100 Nanoparticle Analyzer. The average particle size may be a Dv50 value or a Dn50 value. The particle size is typically a hydrodynamic diameter.

The average particle size may alternatively be measured by scanning electron microscopy (SEM). For instance, the average particle size may be as measured using image analysis of SEM images.

The inorganic compound precursor is a compound suitable for forming the inorganic compound, for instance when dissolved in the solvent. The inorganic compound precursor is typically a silica precursor, a titania precursor or a alumina precursor. The inorganic precursor compound is preferably a silica precursor.

A silica precursor is typically a compound which hydrolyses to produce silica. The silica precursor may for instance be a compound of formula Si(R²)ₓ(OR³)_{y}, where: each R² and each R³ are independently selected from H, C₁₋₆ alkyl, aryl and C₂₋₆ alkenyl; x is 0, 1 or 2; and y is 2, 3 or 4. The sum of x and y is typically 4. Each R² and each R³ is typically independently selected from C₁₋₆ alkyl, for instance from methyl, ethyl, n-propyl, iso-propyl and n-butyl.

An aryl group, as used herein, refers to a monocyclic, bicyclic or polycyclic aromatic ring which contains from 6 to 14 carbon atoms, typically from 6 to 10 carbon atoms, in the ring portion. Examples include phenyl, naphthyl, indenyl, indanyl, anthrecenyl and pyrenyl groups. An C₂₋₆ alkenyl group, as used herein, refers to a C₂₋₆ alkyl group in which one or more carbon-carbon single bonds has been replaced with a carbon-carbon double bonds. Examples include ethenyl, propenyl and butenyl.

The inorganic compound precursor is typically tetraethylorthosilicate (TEOS), tetramethylorthosilicate, tetrapropylorthosilicate or tetrabutylorthosilicate. Preferably the inorganic precursor compound is tetraethylorthosilicate.

In step (b), following addition of the inorganic compound precursor to the composition comprising the solvent and the plurality of polymer nanoparticles, the concentration of the inorganic compound precursor compound is typically from 1.0 mM to 0.1 M. For instance, the concentration of the inorganic compound precursor may be from 0.01 to 0.05 M. If the inorganic compound precursor is a silica precursor, for instance TEOS, the concentration of the silica precursor compound may be from 0.002 to 0.015 ml/ml of the composition comprising the solvent and the plurality of polymer nanoparticles.

In a process according to the invention, the following reagents may be used per 391 ml of solvent in step (a): (i) from 0.1 to 2.0 g resorcinol, preferably from 0.2 to 0.7 g resorcinol; and (ii) from 0.1 to 3.0 mL of 37 wt % formaldehyde in water, preferably from 0.5 to 1.0 mL of 37 wt % formaldehyde in water. In step (b), the concentration per 391 ml of solvent may be from 1.0 to 5.0 mL of tetraethyl orthosilicate, for instance from 2.0 to 4.0 mL of tetraethyl orthosilicate. In step (c), the following amounts of reagents may be used per 391 ml of solvent: (i) from 0.2 to 4.0 g resorcinol, preferably from 1.5 to 2.0 g resorcinol; and (ii) from 0.5 to 6.0 mL of 37 wt % formaldehyde in water, preferably from 2.0 to 3.0 mL of 37 wt % formaldehyde in water.

Addition of the inorganic compound precursor to the composition comprising the solvent and the plurality of polymer nanoparticles produces a composition comprising the solvent and a plurality of inorganic compound-coated polymer nanoparticles. Each inorganic compound-coated polymer nanoparticle typically comprises a core comprising the polymer and a shell comprising the inorganic compound. The average particle size of the plurality of inorganic compound-coated polymer nanoparticles is typically from 120 to 400 nm.

The conditions in step (b) for producing the plurality of inorganic compound-coated polymer nanoparticles (e.g. the silica-coated polymer nanoparticles) may be the same as those required for the Stöber process (Stöber et al, Journal of Colloid and Interface Science. 26 (1): 62-69; 1968).

Step (a) is carried out at a temperature of at least 30° C. The inventors have found that it is also advantageous to control the temperature of step (b), in which the inorganic compound-coated polymer nanoparticles are produced. In particular, it has been found that it is beneficial to cool the reaction mixture (i.e. solvent and polymer nanoparticles) between step (a) and step (b). This can lead to greater control over particle size. Controlling the temperature in step (b) also leads to a desirable “spiky” surface morphology for the hollow inorganic nanoparticles.

Typically, step (b) is carried out at a temperature of no more than 30° C., for instance at a temperature of from 10° C. to 30° C. The temperature may be from 18° C. to 28° C.

The process typically further comprises a step of cooling the composition comprising the solvent and the plurality of polymer nanoparticles in between step (a) and step (b). Typically, the composition comprising the solvent and the plurality of polymer nanoparticles is cooled at an average rate of from 0.5° C./min to 1.0° C./min. The composition comprising the solvent and the plurality of polymer nanoparticles is typically cooled for a time of from 10 minutes to 60 minutes, for instance from 20 to 50 minutes. For instance, the composition comprising the solvent and the plurality of polymer nanoparticles may be cooled from a temperature of from 40° C. to 50° C. to a temperature of from 10° C. to 30° C. over a time of from 20 to 50 minutes.

After addition of the inorganic compound precursor, the coating of the polymer nanoparticles with the inorganic compound is typically allowed to proceed for a time of from 1.0 to 30 minutes. After that period, step (c), addition of additional amounts of the first and second monomers is commenced. After addition of the additional amounts of the first and second monomers, the reaction mixture comprises the first and second monomers as well as the inorganic compound precursor. As a result, the polymer and the inorganic compound are deposited simultaneously on the inorganic compound-coated polymer nanoparticles which leads to the creation of a mesoporous layer of the inorganic compounds where mesopores in the inorganic compound are filled with the polymer. The term “mesoporous” refers to a material comprising mesopores, i.e. pores having widths (i.e. pore sizes) of from 2 nm to 50 nm.

Step (c), adding an additional amount of the first and second monomers to the composition comprising the solvent and the plurality of inorganic compound-coated polymer nanoparticles, is typically carried out from 1 to 30 minutes after step (b), adding a silica precursor compound to the composition comprising the solvent and the plurality of polymer nanoparticles. Preferably, step (c) is carried out from 2 to 10 minutes after step (b).

Step (c) is typically conducted at the same temperature as step (b). For instance, in step (c) the temperature of the composition comprising the solvent and the plurality of inorganic compound-coated polymer nanoparticles is typically no more than 30° C., for instance from 18° C. to 28° C.

Typically, following addition of an additional amount of the first and second monomers to the composition comprising the solvent and the plurality of inorganic compound-coated polymer nanoparticles, the concentration of first monomer is from 2.0 mM to 0.2 M and the concentration of the second monomer is from 2.0 mM to 0.2 M in the composition comprising the solvent, the plurality of inorganic compound-coated polymer nanoparticles and the first and second monomers. The mass of the first monomer (for instance resorcinol) added may for instance be from 1.5 mg/ml to 6.0 mg/ml or from 2.0 mg/ml to 4.0 mg/ml relative to the volume of the reaction mixture as a whole. The volume of the second monomer (for instance formaldehyde) added may for instance be from 0.02 to 0.1 ml of a solution comprising from 20 to 50 wt % of the second monomer/ml of the reaction mixture as a whole. For instance, from 0.2 to 0.6 g of resorcinol may be added for each 80 ml of solvent and from 0.2 to 0.8 ml of 37 wt % aqueous solution of formaldehyde may be added for each 80 ml of solvent.

Typically, the additional amount of first and second monomers are allowed to react for from 1.0 to 4.0 hours. This is the time for which the outer mesoporous layer of the inorganic compound is formed. The mesoporous layer of the inorganic compound forms a surface which may be described as rough or spiky after ultimate removal of the polymer.

Step (c) leads to the production of a plurality of composite nanoparticles. The composite nanoparticles typically comprise: a core comprising the polymer; a shell layer comprising the inorganic compound; and an outer layer comprising the polymer and the inorganic compound. The shell layer typically comprises some pores which, once the polymer has been removed, allow movement of materials from the exterior to the interior of the hollow inorganic nanoparticle.

It has been found that the process of the invention may be carried out at a large scale. For instance, the total volume of the solvent may be at least 500 mL or at least 5 L. Steps (a) to (c) may be conducted in a reaction vessel having a capacity of at least 500 mL or of at least 5 L. The reaction vessel may be a Radleys reactor.

Step (d) comprises heating the plurality of composite nanoparticles to remove the polymer component and thereby produce the plurality of hollow inorganic nanoparticles. Typically, step (d) comprises heating the plurality of composite nanoparticles at a temperature suitable to remove the polymer from composite nanoparticles. For instance, the plurality of composite nanoparticles may be heated at a temperature of from 400° C. to 700° C. or from 500° C. to 600° C. The ramp rate during the calcination step (i.e. the heating in step (d)) is typically from 1° C./min to 20° C./min. It has been found that the ramp rate may be increased without adversely affecting the surface morphology of the hollow inorganic nanoparticles. The ramp rate may be from 6° C./min to 15° C./min.

It has been found that the time required to heat (e.g. calcine) the nanoparticles is less than previously expected. Step (d) typically comprises heating the plurality of composite nanoparticles for a time of less than 4.0 hours. For instance, the plurality of composite nanoparticles may be heated for a time of from 1.0 to 3.0 hours or from 90 to 150 minutes.

Before step (d), the process typically comprises isolating the plurality of composite nanoparticles.

This typically comprises centrifuging the composition comprising the solvent and the composite nanoparticles, for instance at from 3000 to 5000 rpm for from 1 to 20 minutes at a temperature of from 5 to 20° C. During centrifugation, the supernatant is typically removed and additional solvent (e.g. ethanol) is added. Once the plurality of composite nanoparticles have been isolated, they may be dried in air, for instance at room temperature for from 12 to 48 hours.

The total yield of hollow inorganic nanoparticles is typically greater than or equal to 1.0 g per litre of solvent used in steps (a) to (c), for instance greater than or equal to 1.5 g/L.

The plurality of hollow inorganic nanoparticles are typically a plurality of mesoporous hollow inorganic nanoparticles. Each of the hollow inorganic nanoparticles may comprise: a shell comprising an inorganic compound; a volume within the shell which does not comprise the inorganic compound; and disposed on the exterior of the shell, a plurality of protrusions comprising the inorganic compound.

The hollow inorganic nanoparticles typically have a rough or “spiky” surface morphology which contains the plurality of protrusions comprising the inorganic compound. The protrusions of the inorganic compound are volumes of the inorganic compound which extend outwards from the shell comprising the inorganic compound. The protrusions typically increase the surface area of the hollow inorganic nanoparticle. The protrusions on the surface of the shell typically form a further layer of the nanoparticles, which layer is a mesoporous layer comprising the inorganic compound. The thickness of this mesoporous layer (i.e. the length of the protrusions) is typically from 10 nm to 200 nm, for instance from 50 nm to 150 urn. The porosity of the mesoporous layer comprising the inorganic compound typically increases going from the part of the mesoporous layer closest to the shell comprising the inorganic compound to the part of the mesoporous layer closest to the exterior surface of the hollow inorganic nanoparticle.

Typically, the hollow inorganic nanoparticles have an average particle size of from 100 nm to 600 nm, for instance from 120 nm to 400 nm or from 150 nm to 250 nm. The volume within the shell typically has an average diameter of from 50 nm to 500 nm, for instance from 100 to 300 nm. The shell comprising the inorganic compound typically has an average thickness of from 10 nm to 200 nm.

The hollow inorganic nanoparticles have an average particle size of from 150 nm to 250 nm and the volume within the shell may have an average diameter of from 50 nm to 150 nm.

The hollow inorganic nanoparticles are typically useful for formulating and delivering active agents. The process may accordingly further comprise step (e) of treating the plurality of hollow inorganic nanoparticles with an agent to produce a plurality of hollow inorganic nanoparticles loaded with the agent. The agent may be any suitable agent, and is typically an active agent, for instance a hydrophobic active agent. The hollow inorganic nanoparticles can enhance the transport of the active agents to certain locations within a cell or organism. For instance, the hollow inorganic nanoparticles can enhance the transport of nucleic acids to the nucleus of a cell by protecting the nucleic acids during transport through the cell.

Prior to treating the plurality of hollow inorganic nanoparticles with an active agent, it is often desirable to treat the hollow inorganic nanoparticles with a charge modifying agent. The charge modifying agent is typically an amine polymer, for instance a polyamine. Alternatively, the charge modifying agent may be chitosan or a derivative thereof in which the amino group in chitosan is trialkylated, e.g. alkylated with three C₁₋₆ alkyl groups, for instance with three methyl groups (trimethylated). Thus, trimethylchitosan may be employed. Chitosan and its derivatives have been used previously in nonviral gene delivery.

The surface of the hollow inorganic nanoparticles is typically negatively charged and the charge modifying agent is typically a cationic polymer. Use of a cationic polymer allows the hollow nanoparticles to be loaded with a negatively charged agent such as a nucleic acid. The cationic polymer is typically a polyamine, for instance polyethyleneimine (PEI), polymethyleneimine or polyprolyleneimine. The cationic polymer may be a polypeptide, for instance polyarginine, polylysine or polyhistidine. The cationic polymer may be polyainidoamine (PAMAM).

Typically, the charge modifying agent is polyethyleneimine. The polyethyleneimine is typically branched polyethyleneimine. The polyethyleneimine may be linear polyethyleneimine. The polyethyleneimine may have a molecular weight of from 5,000 MW to 40,000 MW, for instance from 10,000 MW to 25,000 MW. The polyethyleneimine typically has a molecular weight of from 5,000 MW to 15,000 MW, for instance about 10,000 MW, which molecular weight is typically a weight-average molecular weight.

The active agent may be a pesticide, a herbicide, a therapeutic agent, a vaccine, a transfection reagent, a nucleic acid or a dye. The pesticide may for instance be spinosad.

The therapeutic agent may be a nucleic acid, for instance a nucleic acid vaccine. The nucleic acid is typically DNA (for instance plasmid DNA) or RNA (for instance mRNA, siRNA, or sRNA). Thus, the nucleic acid may be a DNA vaccine or an RNA vaccine (for instance an mRNA vaccine, or an siRNA vaccine). The nucleic acid may for instance be ovalbumin pDNA, ovalbumin mRNA, HPV pDNA or HPV mRNA. The nucleic acid may be RNA or DNA which encodes luciferase. When the agent is a nucleic acid, treating the hollow inorganic nanoparticles with the nucleic acid is typically conducted in a buffered saline solution. Prior to treating the hollow inorganic nanoparticles with the nucleic acid, the resulting composition may be cooled to a temperature of from 2 to 10° C. for from 1 to 10 hours.

The therapeutic agent may be a small molecule, for instance an antiproliferative compound, an antibiotic compound or an immunotherapeutic compound.

The therapeutic agent may be a protein, for instance it may be a vaccine which comprises a protein.

It has been found that the hollow inorganic nanoparticles can enhance the activity of a therapeutic agent and accordingly that the hollow inorganic nanoparticles have an adjuvant effect. For instance, the hollow inorganic particles can act as an adjuvant by enhancing an immune response following delivery of a vaccine and thereby reducing the amount of vaccine required.

As mentioned above, the surface of the hollow inorganic nanoparticles is typically negatively charged. It can be desirable to enhance the negative charge on the surface of the hollow inorganic nanoparticles by treating the nanoparticles with a acidity modifying component which adds (typically deprotonated) acid groups to the surface of the nanoparticles and thereby increases the negative charge on the surface of the hollow inorganic nanoparticles. This can improve binding of cationic charge modifying agents such as polyethyleneimine to the surface of the nanoparticles. “Binding” includes covalent and non-covalent binding, for instance ionic binding. Typically, the charge modifying agent binds to the acidity-modified surface of the hollow inorganic nanoparticles by an ionic interaction or a van der Waals interaction.

The process may therefore comprise a step of treating the plurality of hollow inorganic nanoparticles with an acidity modifying component (which may also be referred to as an acidic linker) prior to treating the plurality of hollow inorganic nanoparticles with an agent (for instance the charge modifying agent).

The acidity modifying component typically comprises an acidic group having a pKa of less than silica (i.e. a pKa of less than about 4.5). The acidic group may be protonated or deprotonated. Preferably the acidic group is deprotonated as this increases the negative charge on the surface of the hollow nanoparticles. Typically, the acidity modifying component comprises an acidic group which has as a pKa of less than or equal to 3.5. For instance, the acidity modifying component may comprise a phosphonate group, a phosphate group, a sulfate group, a carboxylate group, or an alpha-keto carboxylate group (—C(O)—COO⁻). The acidity modifying component may comprise pyruvate.

The acidity modifying component may be a compound of formula S-R-A where S is a group comprising silicon, R is a divalent organic moiety and A is an acidic group. S is typically a group of formula —Si(alk)ₙ(OH)ₘ where alk is a C₁₋₆ alkyl group, n is from 0 to 3 and OH is from 0 to 3. For instance, S may be —Si(OH)₃. R is typically a C₁₋₆ alkylene group, for instance —(CH₂)ₚ—, where p is an integer from 1 to 6. A is typically a phosphonate group (e.g. —O—P(R^{p})(═O)O⁻, where R^{p} is H or a C₁₋₆ alkyl group), a phosphate group, a sulfate group, a carboxylate group, or an alpha-keto carboxylate group (—C(O)—COO⁻). A is preferably a phosphonate group, for instance methylphosphonate. A may be in the form of the salt of the acidic group, for instance methylphosphonate monosodium or pyruvate monosodium. For instance, S may be trihydroxysilyl, R may be —(CH₂)₃— and A may be a phosphonate group. The silicon-containing group can react with the inorganic material (for instance silica) in the hollow inorganic nanoparticle and add the acidic group to the surface of the hollow inorganic nanoparticle.

The hollow inorganic nanoparticles are typically treated with the acidity modifying agent at a concentration of from 0.005 g/mL to 0.1 g/mL. The temperature of reaction between the acidity modifying agent and the hollow inorganic nanoparticles is typically from 20 to 50° C., for instance from 35 to 45° C. The reaction time is typically from 1 to 5 hours.

The process may comprise a step of treating the plurality of hollow inorganic nanoparticles with a phosphonate linker prior to treating the plurality of hollow inorganic nanoparticles with the agent. In that case, the acidity modifying component is a phosphonate acidity modifying component. Often, for instance, the process comprises a step of treating the plurality of hollow inorganic nanoparticles with a phosphonate linker prior to treating the plurality of hollow inorganic nanoparticles with a charge modifying agent (for instance, polyethyleneimine). The phosphonate linker is typically 1,3-(trihydroxysilyl) propylmethylphosphonate monosodium salt (THPMP).

Often, for instance, the process (i.e. step (e) thereof) comprises: (e1) treating the hollow inorganic nanoparticles with a charge modifying agent; and (e2) treating the hollow inorganic nanoparticles with an active agent. The process, i.e. step (e) thereof, may for instance comprise: (e1) treating the hollow inorganic nanoparticles with an acidity modifying component; (e2) treating the hollow inorganic nanoparticles with a charge modifying agent; and (e3) treating the hollow inorganic nanoparticles with an active agent. The process, i.e. step (e) thereof, may for instance comprise: (e1) treating the hollow inorganic nanoparticles with a phosphonate linker; (e2) treating the hollow inorganic nanoparticles with a charge modifying agent; and (e3) treating the hollow inorganic nanoparticles with an active agent. The hollow inorganic nanoparticles may for instance be hollow silica nanoparticles. The phosphonate linker may for instance be THPMP. The charge modifying agent may for instance be as further defined above, for instance a polyamine, e.g. PEI, or chitosan or a derivative thereof. The active agent may also be as further defined above, for instance it may be a nucleic acid, protein or small molecule, and may for instance be a nucleic acid (e.g. DNA or RNA) vaccine, or a protein or peptide vaccine.

It has been found that the hallow inorganic nanoparticles can be loaded with the agent, for instance the charge modifying agent, quickly. The plurality of hollow inorganic nanoparticles may therefore be treated with the agent, e.g. the charge modifying agent, for less than 60 minutes or less than 15 minutes, for instance from 30 seconds to 15 minutes. For instance, phosphonate linked hollow inorganic nanoparticles may be treated with polyethyleneimine for from 1 to 10 minutes. Often, however, it is preferable to treat the hollow inorganic nanoparticles with the charge modifying agent for at least one hour, for instance from 2 to 10 hours. The hollow inorganic nanoparticles may be treated with the charge modifying agent at a temperature of from 20 to 30° C.

The invention also provides a plurality of hollow inorganic nanoparticles obtainable by a process according to the invention.

The invention further provides a plurality of hollow inorganic nanoparticles, wherein each of the hollow inorganic nanoparticles comprises: a shell comprising an inorganic compound; a volume within the shell which does not comprise the inorganic compound; and disposed on the exterior of the shell, a plurality of protrusions comprising the inorganic compound. The particle size of the plurality of hollow inorganic nanoparticles is typically from 100 to 500 nm. The hollow inorganic nanoparticles may be as described above. The hollow inorganic nanoparticles are typically hollow silica nanoparticles.

The invention further provides a plurality of hollow inorganic nanoparticles, wherein each of the hollow inorganic nanoparticles comprises: a shell comprising an inorganic compound; a volume within the shell which does not comprise the inorganic compound; and disposed on the exterior of the shell, a plurality of protrusions comprising the inorganic compound, and wherein the hollow inorganic nanoparticles further comprise a plurality of acidic groups bound to the inorganic compound. The acidic group is typically a phosphonate group (—O—P(R^{p})(═O)O⁻, where R^{p} is H or a C₁₋₆ alkyl group), a phosphate group, a sulfate group, a carboxylate group, or an alpha-keto carboxylate group (—C(O)—COO⁻). The acid groups may for instance be a methylphosphonate group. The hollow inorganic nanoparticles comprising acidic groups bound to the surface may be obtainable by treating the hollow inorganic nanoparticles with a compound of formula S-R-A as defined above. The acidic groups are typically negatively charged. For instance, the acidic groups may be in the forms of salts, where the counterion is typically an alkali metal cation such as sodium.

As discussed above, the presence of acidic groups such as phosphonate on the surface of the inorganic nanoparticle advantageously increases the negative the charge at the surface of the hollow inorganic nanoparticle which can in turn improve binding of a charge modifying agent such as polyethyleneimine to the nanoparticle.

The average particle size of the plurality of hollow inorganic nanoparticles is typically from 150 to 350 nm, for instance from 160 to 250 nm. The average particle size of the plurality of hollow inorganic nanoparticles may be from 160 to 200 nm. The particle sizes are typically as measured using dynamic light scattering, as discussed above. The particle sizes may be as measured by image analysis of SEM images.

The plurality of hollow inorganic nanoparticles according to the invention may be highly monodisperse. Typically, the polydispersity index (PDI, also known as the dispersity index) of the plurality of hollow inorganic nanoparticles is less than or equal to 0.3, less than or equal to 0.15, less than or equal to 0.1 or less than or equal to 0.05. The dispersity index can be calculated as the ratio of the quadratic average (i.e., average value of squares of measured diameters, d), and square of arithmetic average of measured diameters. The calculations for the dispersity index may be as defined in the ISO standard document 13321:1996 E and ISO 22412:2008.

For instance, the hollow inorganic nanoparticles according to the invention or produced by the process of the invention may have an average particle size (for instance as measured by SEM) of from 150 to 200 nm and a polydispersity index of no more than 0.15. The hollow inorganic nanoparticles may have an average particle size of from 150 to 250 nm and a polydispersity index of no more than 0.25.

The hollow inorganic nanoparticles typically have high surface areas. For instance, the plurality of the hollow inorganic nanoparticles may have a BET surface area of at least 120 cm²/g, for instance at least 150 cm²/g. The inorganic nanoparticles may have a BET surface area of at least 140 cm²/g. The plurality of hollow inorganic nanoparticles may have a mean particle size of from 160 to 250 nm and a BET surface area of at least 120 cm²/g. The BET surface area may for instance be measured using the ISO 9277 standard. The BET surface area may be measured based on adsorption and desorption of nitrogen.

The invention also provides a composition comprising a plurality of hollow inorganic nanoparticles according to the invention and an agent. The agent may be as defined herein. The agent is typically bound to the hollow inorganic nanoparticles, for instance by a phosphonate linker; this is particularly the case when the agent comprises a charge modifying agent such as polyethyleneimine. The phosphonate linker may be 1,3-(trihydroxysilyl) propylmethylphosphonate monosodium salt (THPMP).

The agent is typically a hydrophobic active agent. For instance the agent may be a pesticide, a herbicide, a therapeutic agent, a vaccine, a charge modifying agent, a transfection reagent, an agent comprising DNA, or a dye.

Typically, the agent is a change modifying agent which is polyethyleneimine. The polyethyleneimine is typically branched polyethyleneimine. The polyethyleneimine may be liner polyethyleneimine. The polyethyleneimine may have a molecular weight of from 5,000 MW to 40,000 MW, for instance from 10,000 MW to 25,000 MW. The polyethyleneimine typically has a molecular weight of from 5,000 MW to 15,000 MW, for instance about 10,000 MW, which molecular weight is typically a weight-average molecular weight.

Typically, the plurality of hollow inorganic nanoparticles comprises at least 1.0% by weight of the charge modifying agent. For instance, the plurality of hollow inorganic nanoparticles may comprise at least 2.0% by weight or at least 5.0% by weight of the charge modifying agent. The plurality of hollow inorganic nanoparticles may comprise from 6.0 to 15% by weight of the charge modifying agent, for instance polyethyleneimine.

The plurality of hollow inorganic nanoparticles may be functionalised with a phosphonate linker, e.g. THPMP.

Preferably, the composition comprises a charge modifying agent and an active agent. The charge modifying agent is typically bound to the hollow inorganic nanoparticles. For instance, it may be bound to the hollow inorganic nanoparticles by a phosphonate linker, e.g. THPMP. The charge modifying agent may be as further defined herein and is typically an amine polymer, for instance a polyamine. Alternatively, the charge modifying agent may be chitosan or a derivative thereof in which the amino group in chitosan is trialkylated, e.g. alkylated with three C₁₋₆ alkyl groups, for instance with three methyl groups (trimethylated). Thus, trimethylchitosan may be employed.

Chitosan and its derivatives have been used previously in nonviral gene delivery. The charge modifying agent may be a polypeptide such as polyhistidine, polylysine or polyarginine.

The surface of the hollow inorganic nanoparticles is typically negatively charged and the charge modifying agent is typically a cationic polymer. Use of a cationic polymer allows the hollow nanoparticles to be loaded with a negatively charged agent such as a nucleic acid. The cationic polymer is typically a polyamine, for instance polyethyleneimine (PEI), polymethyleneimine or polyprolyleneimine. Typically, the charge modifying agent is polyethyleneimine. The polyethyleneimine is typically branched polyethyleneimine. The polyethyleneimine typically has a molecular weight of from 5,000 MW to 15,000 MW, for instance about 10,000 MW, which molecular weight is typically a weight-average molecular weight. The active agent may be a pesticide, a herbicide, a therapeutic agent, a vaccine, a transfection reagent, a nucleic acid or a dye. The pesticide may for instance be spinosad. The active agent is typically bound to the charge modifying agent, e.g. electrostatically (an example of this being negatively charged nucleic acid bound to cationic polyamine, e.g. PEI, or to chitosan or a derivative of chitosan). Thus the therapeutic agent may be a nucleic acid, for instance a nucleic acid vaccine. The nucleic acid is typically DNA (for instance plasmid DNA) or RNA (for instance mRNA, siRNA, or sRNA). Thus, the nucleic acid may be a DNA vaccine or an RNA vaccine (for instance an mRNA vaccine, or an siRNA vaccine). The therapeutic agent may be a small molecule, for instance an antiproliferative compound, an antibiotic compound or an immunotherapeutic compound. The therapeutic agent may be a protein, for instance it may be a vaccine which comprises a protein.

The weight ratio of the active agent (for instance DNA or RNA) to the hollow inorganic nanoparticles is typically from 1:2 to 1:100 (active agent:nanoparticles), for instance from 1:5 to 1:50 or from 1:20 to 1:50.

Preferably, the composition comprises a charge modifying agent and a nucleic acid. The nucleic acid is typically DNA (for instance plasmid DNA) or RNA (for instance mRNA, siRNA, or sRNA). For instance, the composition may comprise polyethyleneimine (PEI) and the nucleic acid, for instance PEI and plasmid DNA.

The charge modifying agent, e.g. PEI, is typically bound to the hollow inorganic nanoparticles by a phosphonate linker, for instance by 1,3-(trihydroxysilyl) propylmethylphosphonate monosodium salt (THPMP). The binding between PEI and the phosphonate group added to the surface of the hollow inorganic nanoparticles by the THPMP is typically ionic bonding.

The composition of the invention may comprise the hollow inorganic nanoparticles at a concentration of greater than or equal to 10 μg/mL, greater than or equal to 40 μg/mL or greater than or equal to 60 μg/mL.

The composition of the invention is generally a pharmaceutical composition. Preferred pharmaceutical compositions are sterile and pyrogen free. The composition of the invention often, therefore, further comprises a pharmaceutically acceptable carrier or diluent. For example, a solution for injection or infusion may contain as carrier, for example, sterile water or may for instance be in the form of a sterile, aqueous, isotonic saline solution. A solid oral form, on the other hand, may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non toxic and pharmacologically inactive substances used in pharmaceutical formulations. Such pharmaceutical preparations may be manufactured in known manner, for example, by means of mixing, granulating, tableting, sugar coating, or film coating processes. Liquid dispersions for oral administration may be syrups, emulsions and suspensions. The syrups may contain as carriers, for example, saccharose or saccharose with glycerine and/or mannitol and/or sorbitol. Suspensions and emulsions may contain as carrier, for example a natural gum, agar, sodium alginate, pectin, methylcellulose, carboxymethylcellulose, or polyvinyl alcohol. The suspension or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

The invention also provides a composition as defined herein for use in the treatment of the human or animal body by therapy. In this context treatment includes the amelioration and prevention of a disease. The active agent may for instance be a vaccine and the composition may be for use in the prevention of a disease in a patient by immunising the patient against the disease using the vaccine.

The invention also provides a method for the treatment of a disease, which method comprises administering a therapeutically effective amount of a composition as defined herein to a subject in need thereof. The subject may be a mammal, and is typically a human patient. Again, the term “treatment” here, includes amelioration or prevention of the disease. The active agent in the composition may for instance be a vaccine. The treatment may for example comprise prevention of the disease in the subject by immunising the subject against the disease using the vaccine. A therapeutically effective amount of a composition of the invention is administered to the subject, and this amount may readily be determined by the skilled person, according to the activity of the particular agent being employed in the composition, and the age, weight and conditions of the subject to be treated, the type and severity of the disease and the frequency and route of administration.

The diseases which may be treated by the nanoparticles include cancer, bacterial infection, viral infection and immune disorders. The treatment may for instance comprise immunotherapy, for instance the treatment of cancer by immunotherapy.

The invention also provides a plurality of hollow inorganic nanoparticles according to the invention for use as an adjuvant in the treatment of the human or animal body by therapy. Accordingly, the plurality of hollow inorganic nanoparticles may be used in a method of increasing the effect of a therapeutic agent. For instance, the invention may provide a method of increasing the effect of a therapeutic agent by co-administering the therapeutic agent with a plurality of the hollow inorganic nanoparticles.

The therapeutic agent is typically a vaccine, a nucleic acid or a chemotherapeutic agent. The plurality of hollow inorganic nanoparticles may accordingly act as a vaccine adjuvant. The plurality of hollow inorganic nanoparticles may be used in a method of increasing an immune response to a vaccine. The invention may provide a composition comprising the plurality of hollow inorganic nanoparticles as an adjuvant and a therapeutic agent such as a vaccine. The plurality of hollow inorganic nanoparticles may cause an immune response when administered without an active agent. The invention accordingly provides the plurality of hollow inorganic nanoparticles for use in a method of causing an immune response.

The plurality of hollow inorganic nanoparticles may be for use as an adjuvant in the treatment of cancer, for instance in the treatment of cancer by immunotherapy. The plurality of hollow inorganic nanoparticles may be for use in a method of treating cancer by co-administering a chemotherapeutic agent with a plurality of the hollow inorganic nanoparticles.

The invention also provides a method of transfecting a nucleic acid into a cell, the method comprising treating the cell with a composition according to the invention. The composition may comprise the plurality of hollow inorganic nanoparticles and a nucleic acid. The cell may be a human or non-human cell. The cell may be a cell from the CT26, HCT116 or HEK293 cell lines. The method of transfecting a nucleic acid into a cell may be conducted in vitro for instance in the cell lines mentioned. The composition according to the invention may alternatively be use for transfecting a nucleic acid into a cell in the human or animal body.

It has unexpectedly been found that the hollow inorganic nanoparticles of the invention not only transfect cells very successfully but at the same time “wake up” the immune system (i.e. act as an adjuvant) to stimulate an advantageous immune response. Accordingly, the invention also provides a method of transfecting a nucleic acid into a cell, the method comprising treating the cell with a composition according to the invention, which composition comprises the plurality of hollow inorganic nanoparticles and a nucleic acid, and thereby transfecting the cell with the nucleic acid and stimulating an immune response. Advantageously, this allows the active agent-loaded SiNP to act as both a vehicle for delivering the active agent (e.g. a vaccine) and an adjuvant. This allows for simplified vaccine compositions comprising adjuvants.

The invention also provides a method for controlling pests at a locus, which method comprises exposing the locus to a composition as defined herein. The locus is typically a crop or a plant. The pest may for instance be an insect.

The invention is described in more detail by the following Examples.

### EXAMPLES

### Example 1—Synthesis of Hollow Silica Nanoparticles (SiNPs)

Materials

The materials used for the silica nanoparticle synthesis are given in Table

TABLE 1
Reagents used in silica nanoparticle synthesis

| Reagent | Grade |
|---|---|
| Ethanol | 98% |
| Water | De-ionised |
| Ammonium hydroxide | 28-30% NH₃ basis |
| Resorcinol | BioXtra > 99% |
| Formaldehyde | 37 wt % in H₂O |
| Tetraethyl orthosilicate (TEOS) | Reagent grade 98% |

Methods

Protocol: Silica Nanoparticle Synthesis, 100 mL Scale

The following protocol was used as the basis for the experiments set out below. It should be noted that in this protocol the two monomers are contacted at ambient temperature.

A 500 mL Duran bottle was treated with ethanol (70 mL), water (10 mL) and ammonium hydroxide (3 mL) and stirred (lid on) at ˜350 rpm on a stirrer hotplate for 15 minutes. Resorcinol (0.2 g) and formaldehyde (0.28 mL) were added and the solution stirred (lid on) for 6 hours at ˜350 rpm at ambient temperature. Tetraethyl orthosilicate (0.6 mL) was added and the mixture stirred (lid on) for 6 minutes. Additional resorcinol (0.4 g) and formaldehyde (0.56 mL) were added and the solution stirred (lid on) for a further 2 hours.

The reaction mixture was transferred to 2 centrifuge tubes and centrifugation carried out at 4700 RPM for 5 minutes at 10° C. Supernatant was removed, fresh ethanol added to each tube and centrifugation repeated using 2×40 mL of ethanol. Supernatant as removed and the crude sample transferred into a ceramic dish. Ethanol (5 mL) was used to aid the transfer. The crude sample was dried in air at ambient temperature for 36 hours. Finally the sample was calcined, start temperature: 33° C., ramping temperature: 2° C./min, target temperature: 550° C., holding time: 2 hours. The final silica nanoparticles were obtained as a white or off-white solid.

Silica Nanoparticle Synthesis, 500 mL and 5 L Scale

Reactions were carried out in either a 500 mL or 5 L Radley's reactor equipped with an angled 4-bladed propeller and data logging capability for temperature, pH and stirrer speed. The procedure used was as described for “Protocol: Silica nanoparticle synthesis, 100 mL scale”, but reagent quantities and reaction conditions were scaled and varied, as described in Tables 2 and 3.

Silica Nanoparticle Synthesis, 10 L Scale

Reactions were out in 20 L Radley's reactor equipped with an angled 4-bladed propeller and date logging capability for temperature, pH, conductivity, stirrer speed and torque. The procedure used was as described for “Protocol: Silica nanoparticle synthesis, 100 mL scale”, but reagent quantities and reaction conditions were scaled and varied accordingly, as described in tables 2 and 3. For the 10 L scale reaction addition safety measures were applied to the process as described below.

Analysis of Solution Turbidity Using UV Vis Spectroscopy

Samples were taken periodically and analysed without dilution for solution turbidity between 200 and 700 nm, using an Avantes UV-Vis spectrometer, 1 cm path length cell. Following analysis the sample was returned to the reactor vessel.

Analysis of Particle Size Using Dynamic Light Scattering

Samples were taken periodically and analysed without dilution to observe particle growth during the process. Particle size was measured using dynamic light scattering with a Horiba SZ-100 Nanoparticle analyser. Following analysis the sample was returned to the reactor vessel.

Scanning Electron Microscopy

Scanning electron microscopy was used to image all batches using a Hitachi SU8230. When using the scanning electron microscope, initially particles were coated with a 20 nm chromium layer prior to imaging. However later analysis of uncoated particles, carried out at low voltage to prevent charging, provided a more representative indication of surface morphology.

Analysis of Silica Nanoparticle Calcination Using Thermogravimetric Analysis

Thermogravimetric analysis was used to study mass loss from an example batch of silica nanoparticles. A ramp rate of 2° C./min from ambient temperature to 550° C. (in air) was used followed by a hold at 550° C. for 5 hours. Variations to the calcination process were also studied, as described below.

TABLE 2
Reagent quantities for silica nanoparticle synthesis

| | | | | | | SNP | | SNP | SNP | SNP |
|---|---|---|---|---|---|---|---|---|---|---|
| | SNP | SNP | SNP | SNP | SNP | 0005 | SNP | 0006 | 0006 | 0006 |
| Reagents | 0001 | 0002 | 0003* | 0004* | 0005* | v2 | 0006 | II | III | IV |
| Ethanol (mL) | 70 | 70 | 330 | 330 | 330 | 330 | 330 | 330 | 330 | 330 |
| Water (mL) | 10 | 10 | 47 | 47 | 47 | 47 | 47 | 47 | 47 | 47 |
| Ammonium | 3 | 3 | 14 | 14 | 14 | 14 | 14 | 14 | 14 | 14 |
| hydroxide | | | | | | | | | | |
| (mL) | | | | | | | | | | |
| Resorcinol (g) | 0.2018 | 0.2030 | 0.8662 | 0.8869 | 0.8658 | 0.7075 | 0.7081 | 0.7081 | 0.7070 | 0.7072 |
| [1^{st} addition] | | | | | | | | | | |
| Formaldehyde (mL) | 0.2814 | 0.2816 | 1.3 | 1 | 1 | 1 | 1 | 1 | 1 | 1 |
| [1^{st} addition] | | | | | | | | | | |
| Tetraethyl | 0.6004 | 0.6003 | 2.8 | 2.8 | 2.8 | 2.8 | 2.8 | 2.8 | 2.8 | 2.8 |
| orthasilicate | | | | | | | | | | |
| (mL) | | | | | | | | | | |
| Resorcinol (g) | 0.3995 | 0.4020 | 1.7286^{E} | 1.7886^{E} | 1.7290^{E} | 1.8868^{E} | 1.8871^{E} | 1.8876^{E} | 1.8877^{E} | 1.8869^{E} |
| [2^{nd} addition] | | | | | | | | | | |
| Formaldehyde (mL) | 0.5610 | 0.5604 | 2.6 | 2.6 | 2.6 | 2.6 | 2.6 | 2.6 | 2.6 | 2.6 |
| [2^{nd} addition] | | | | | | | | | | |
| SiNP yield (g) | Not | 0.1298 | 0.2209 | 0.5578 | 0.5404 | 0.5164 | 0.3531 | 0.5508 | 0.4465 | 0.5773 |
| | measured | | | | | | | | | |
| Solid product | NA | 1.5639 | 0.5650 | 1.4266 | 1.3821 | 1.3207 | 0.9031 | 1.4087 | 1.1419 | 1.4765 |
| (g/litre) | | | | | | | | | | |
| Solid yield | NA | 80 | 29 | 74 | 72 | 69 | 47 | 73 | 59 | 77 |
| (%) | | | | | | | | | | |

| | | SNP | SNP | | SNP | | SNP | | | SNP |
|---|---|---|---|---|---|---|---|---|---|---|
| | SNP | 0007 | 0007 | SNP | 0008 | SNP | 0009 | SNP | SNP | 0011 |
| Reagents | 0007 | II | V | 0008 | II | 0009 | II | 0010 | 0011 | II |
| Ethanol (mL) | 330 | 330 | 330 | 3000 | 3300 | 330 | 330 | 330 | 8230 | 8230 |
| Water (mL) | 47 | 47 | 47 | 430 | 470 | 47 | 47 | 47 | 1178 | 1178 |
| Ammonium | 14 | 14 | 14 | 129 | 140 | 14 | 14 | 14 | 350 | 350 |
| hydroxide (mL) | | | | | | | | | | |
| Resorcinol (g) | 0.5136 | 0.5140 | 0.5142 | 4.6738 | 5.1357 | 0.3421 | 0.3421 | 0.5136 | 12.8732 | 12.8713 |
| [1^{st} addition] | | | | | | | | | | |
| Formaldehyde (mL) | 0.7258 | 0.7254 | 0.7268 | 6.6 | 7.2 | 0.4773 | 0.4773 | 0.7260 | 18 | 18 |
| [1^{st} addition] | | | | | | | | | | |
| Tetraethyl | 2.8 | 2.8 | 2.8 | 25 | 28 | 2.8 | 2.8 | 2.8 | 70 | 70 |
| orthosilicate | | | | | | | | | | |
| (mL) | | | | | | | | | | |
| Resorcinol (g) | 1.8873^{E} | 1.8868 | 1.8860 | 17.1693 | 18.8686 | 1.8859 | 0.3420 | 1.8872 | 47.2967 | 47.2842 |
| [2^{nd} addition] | | | | | | | | | | |
| Formaldehyde (mL) | 2.6 | 2.6 | 2.6 | 26 | 29 | 2.6 | 0.4770 | 2.6 | 66 | 66 |
| [2^{nd} addition] | | | | | | | | | | |
| SiNP yield (g) | 0.4739 | 0.5400 | 0.565 | 5.6407 | 5.5956 | 0.4927 | 0.5298 | 0.6078 | 16.9818 | 16.9547 |
| Solid product | 1.2120 | 1.3812 | 1.4450 | 1.5849 | 1.4311 | 1.2601 | 1.3550 | 1.5545 | 1.7403 | 1.7375 |
| (g/litre) | | | | | | | | | | |
| Solid yield (%) | 63 | 72 | 75 | 84 | 74 | 65 | 70 | 81 | 90 | 90 |

| | SNP | SNP | | SNP | SNP | SNP |
|---|---|---|---|---|---|---|
| | 0009 | 0009 | SNP | 0012 | 0012 | 0012 |
| Reagents | III | IV | 0012 | II | III | IV |
| Ethanol (mL) | 330 | 330 | 330 | 330 | 330 | 330 |
| Water (mL) | 47 | 47 | 47 | 47 | 47 | 47 |
| Ammonium hydroxide | 14 | 14 | 14 | 14 | 14 | 14 |
| (mL) | | | | | | |
| Resorcinol (g) | 0.3425 | 0.5874 | 3.5374 | 3.5378 | 3.5360 | 0.5127 |
| [1^{st} addition] | | | | | | |
| Formaldehyde (mL) | 0.4779 | 0.7650 | 5 | 5 | 5 | 0.7230 |
| [1^{st} addition] | | | | | | |

*Higher amount of resorcinol added in the experiment
^{E}Ethanol (40 mL) used to aid transfer to the reactor

TABLE 3
Process conditions for silica nanoparticle synthesis

| | | | | | Spike | | Spike |
|---|---|---|---|---|---|---|---|
| | Recipe | Scale of | Stirring | Polymerisation | Growth | | Growth |
| | Used | Reaction | Rate | Temperature | Temperature | Polymerisation | Time |
| Experiment | (nm) | (mL) | (RPM) | (° C.) | (° C.) | Time (min) | (min) |
| SNP 0006 | 180 | 400 | 350 | 45 | 25 | 153 | 107 |
| SNP 0006 | 180 | 400 | 350 | 45 | 25 | 123 | 153 |
| II | | | | | | | |
| SNP 0006 | 180 | 400 | 250 | 45 | 25 | 117 | 128 |
| III | | | | | | | |
| SNP 0006 | 180 | 400 | 350 | 45 | 25 | 69 | 137 |
| IV | | | | | | | |
| SNP 0007 | 180 | 400 | 350 | 45 | 25 | 133 | 128 |
| SNP 0007 | 180 | 400 | 350 | 45 | 25 | 174 | 130 |
| II | | | | | | | |
| SNP 0007 | 180 | 400 | 350 | 45 | 25 | 126 | 122 |
| V | | | | | | | |
| SNP 0008 | 180 | 4000 | 250 | 45 | 25 | 141 | 136 |
| SNP 0008 | 180 | 4000 | 250 | 45 | 25 | 160 | 125 |
| II | | | | | | | |
| SNP 0009 | 130 | 400 | 350 | 45 | 25 | 260 | 125 |
| SNP 0009 | 130 | 400 | 350 | 45 | 25 | 200 | 145 |
| II | | | | | | | |
| SNP 0010 | 130 | 400 | 350 | 45 | 25 | 320 | 65 |
| SNP 0011 | 180 | 10000 | 160 | 45 | 25 | 134 | 143 |
| SNP 0011 | 180 | 10000 | 160 | 45 | 25 | 133 | 125 |
| II | | | | | | | |

| | | Scale of | | | |
|---|---|---|---|---|---|
| | Recipe | Reaction | Stirring | Polymerisation | Polymerisation |
| Experiment | Used (nm) | (mL) | Rate (RPM) | Temperature (° C.) | Time (min) |
| SNP 0009 | 130 | 400 | 350 | 45 | 120 |
| III | | | | | |
| SNP 0009 | 150 | 400 | 350 | 45 | 105 |
| IV | | | | | |
| SNP 0012 | 180 | 400 | 350 | 45 | 136 |
| SNP 0012 | 180 | 400 | 350 | 45 | 231 |
| II | | | | | |
| SNP 0012 | 180 | 400 | 350 | 45 | 59 |
| III | | | | | |
| SNP 0012 | 180 | 400 | 350 | 45 | 110 |
| IV | | | | | |

Results and Discussion

SiNP001 and SiNP002 (Reference Examples)

Two preparations were carried out at 100 mL scale, targeting a SiNP particle size of 330 nm. Both reactions were monitored and images taken throughout the process. Turbidity measurements were also performed during synthesis SiNP001 using a UV-vis spectrometer, with the increase turbidity observed correlating well with visual observations.

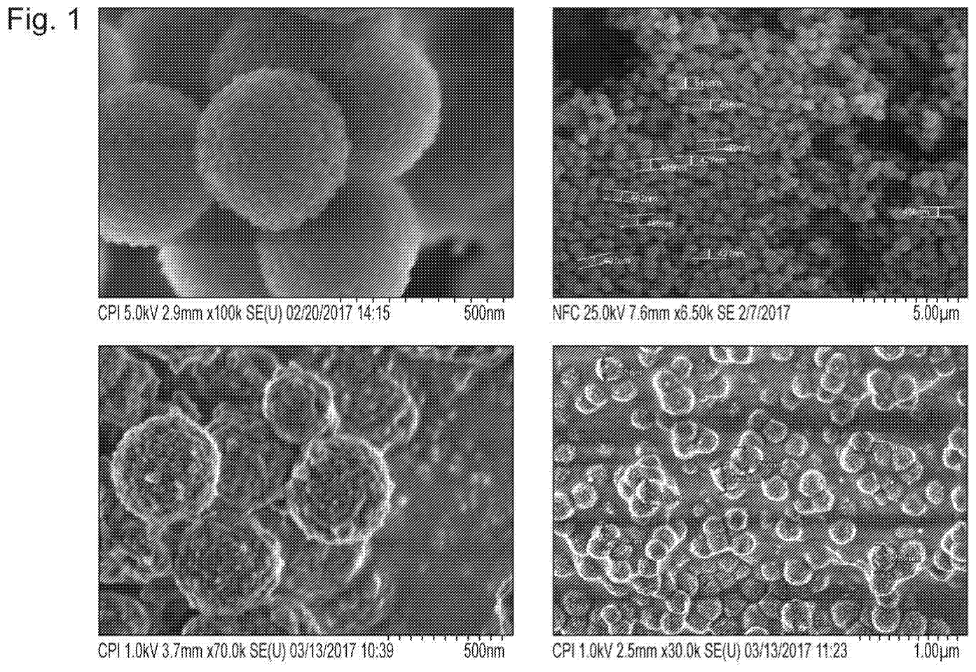

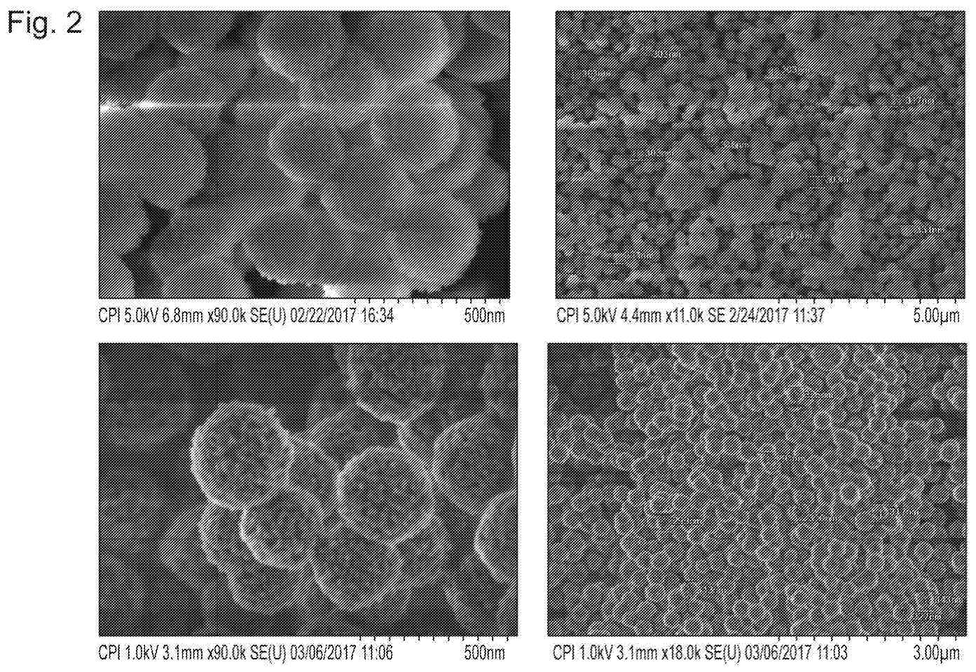

Scanning electron microscopy was used to image both chromium-coated and uncoated particles. Imaging of uncoated particles, at lower voltage to prevent charging, provides a better representation of surface morphology. Application of a chromium layer can lead to masking of surface structure, false augmentation of particle size and in many cases lead to particle agglomeration. Images for particles produced in SiNP001 and SiNP002 preps are shown in FIGS. 1 and 2, respectively.

The particles produced in preps SiNP001 and SiNP002 have a mean particle size of 242 and 300 nm, respectively. Surface morphology is spiky in both cases.

Following successful replication of the prior art synthesis in prep SiNP002, the reaction was scaled-up to 500 mL using a Radley's reactor. In addition to scale the Radley's set up offers a number of advantages over a stirrer hotplate arrangement, including precise control of temperature and stirrer speed, and the ability to monitor process conditions (stirrer speed, temperature and pH). A number of syntheses have been carried out to date and these are summarised below. The outcome of each synthesis is discussed in detail in the following sections.

SiNP003-180 nm Target Particle Size, 25° C. Throughout, 350 rpm Stirrer Speed (Reference Examples)

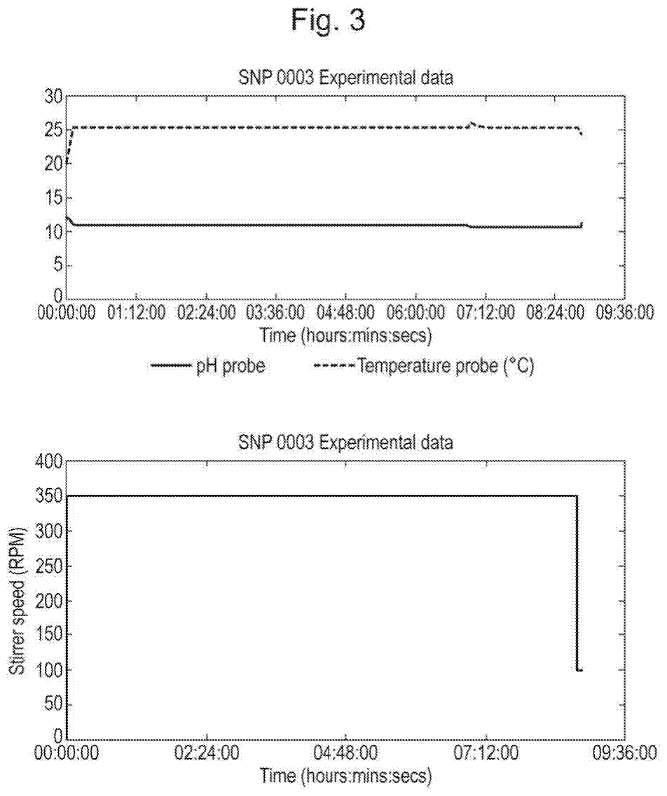

The target particle size for the current programme of work is 180 nm, hence the first scale up prep targeted a particle size in this area. Monitoring of the reaction showed consistency in reaction temperature, pH and stirrer speed, FIG. 3.

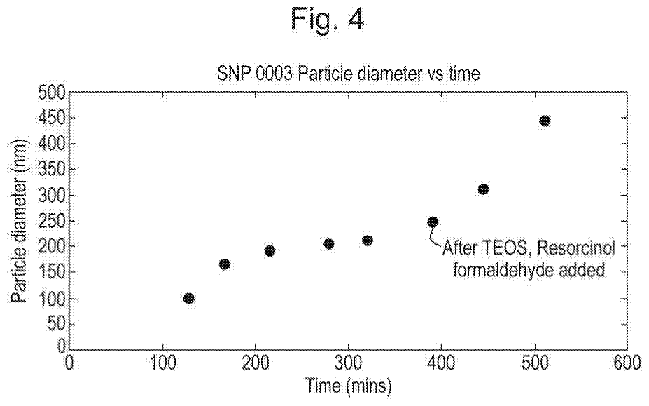

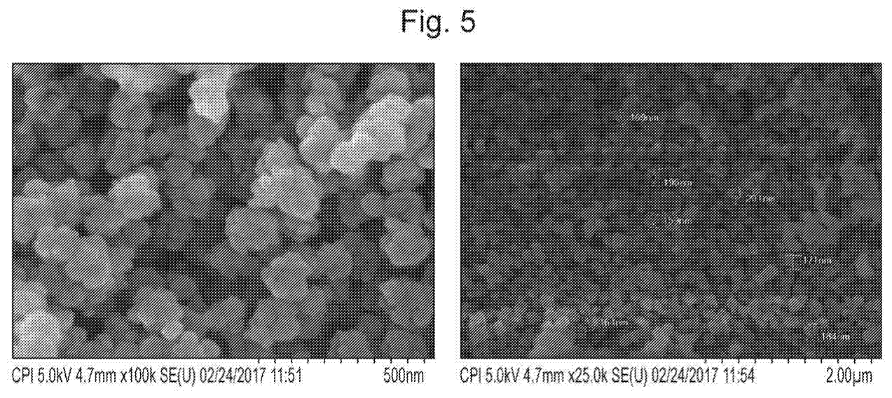

During the course of the synthesis samples of the reaction mixture were taken for particle size analysis using dynamic light scattering. FIG. 4 shows evolution of particle size to a plateau of ˜200 nm after approximately 4 hours, followed by rapid increase in particle size upon addition of the silica shell. However, SEM imaging of the final, calcined SiNP shows a difference in particle size between the techniques. The difference may be due to changes in the refractive index of the particle upon addition of the silica shell, leading to anomalously high values of particle size using DLS. Images of coated and uncoated particles are shown in FIG. 5. Some agglomeration of particles is observed and the surface topology is difficult to determine. Prep SiNP003 resulted in a slight under dose of resorcinol during synthesis of the resorcinol formaldehyde core, which may explain the slightly smaller than expected mean particle size (168 nm) obtained versus the desired 180 nm. This small deviation from the target weight of resorcinol has an effect on particle size which is likely to be more pronounced at this small scale and will become less significant as the scale is increased. Other factors which are likely to have an influence on particle size, and also the agglomeration observed are stirrer speed and type. Stirring using propeller blades general results in significantly better mixing than magnetic stirrers, hence smaller particle size may be favoured by a slower stirrer speed resulting in fewer reagent and particle collisions.

SiNP004—180 nm Target Particle Size, 35° C. Throughout, 350 rpm Stirrer Speed

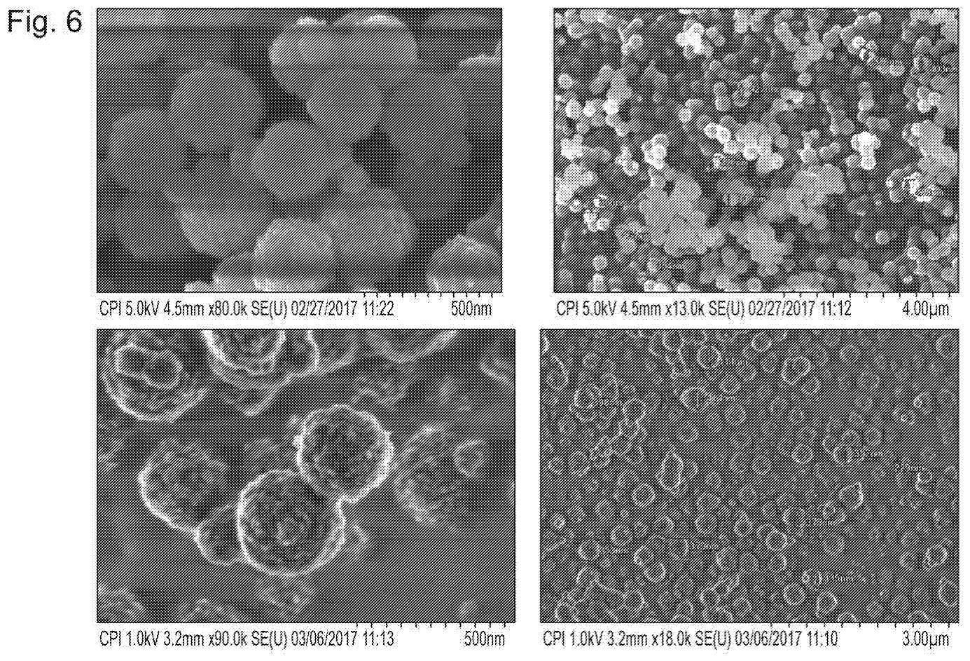

In order to shorten the time required for synthesis of 180 nm SiNP the temperature for SiNP synthesis was increased from 25 to 35° C. Resorcinol was slightly overdosed during formation of the RF (resorcinol-formaldehyde) core. The result of increasing reaction temperature is two-fold. Firstly the particles obtained are significantly larger (mean particle diameter 367 nm), which likely results from faster reaction kinetics in formation of both the core and shell of the particle. The distribution of particles is also bi-modal, with the smaller particles likely attributed to self-condensation of silica in addition to the desired addition of silica to the RF core to generate the spiky structure, FIG. 6.

However, the surface morphology of the particles appears to be ‘spikier’ than in previous preps suggesting use of higher temperature for core, but not shell formation could be promising.

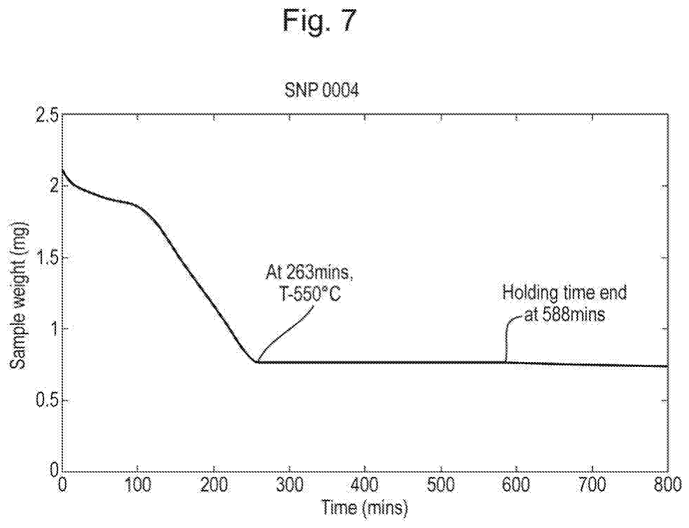

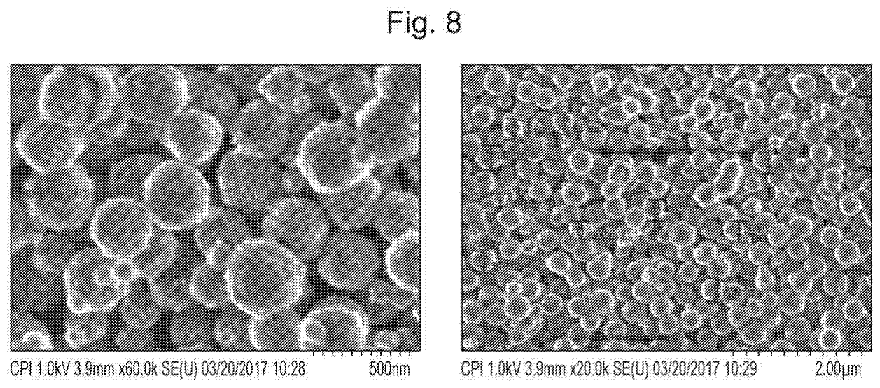

In addition the process of calcination was investigated using TGA. No significant mass loss was observed upon holding at 550° C., FIG. 7. In addition calcination of particles was carried out for 14 hours and compared to a shortened calcination regime (ramp to 550° C. followed by 2 hour hold). No obvious difference in surface morphology was observed hence duration of the calcination regime can be shortened without adversely affecting particle morphology (FIG. 8).

SiNP005—180 nm Target Particle Size, 35° C. RF Core Polymerisation, 25° C. Shell Formation, 350 rpm Stirrer Speed

The bimodal particle size distribution observed in SiNP004 is believed to result from formation of solid silica nanospheres in addition to hollow spiky nanoparticles. In order to circumvent formation of the undesirable silica particles, a prep was carried out using a polymerisation temperature of 35° C. to form the particle core, then a lower temperature (25° C.) to form the spiky silica shell. Resorcinol was slightly overdosed during formation of the RF core.

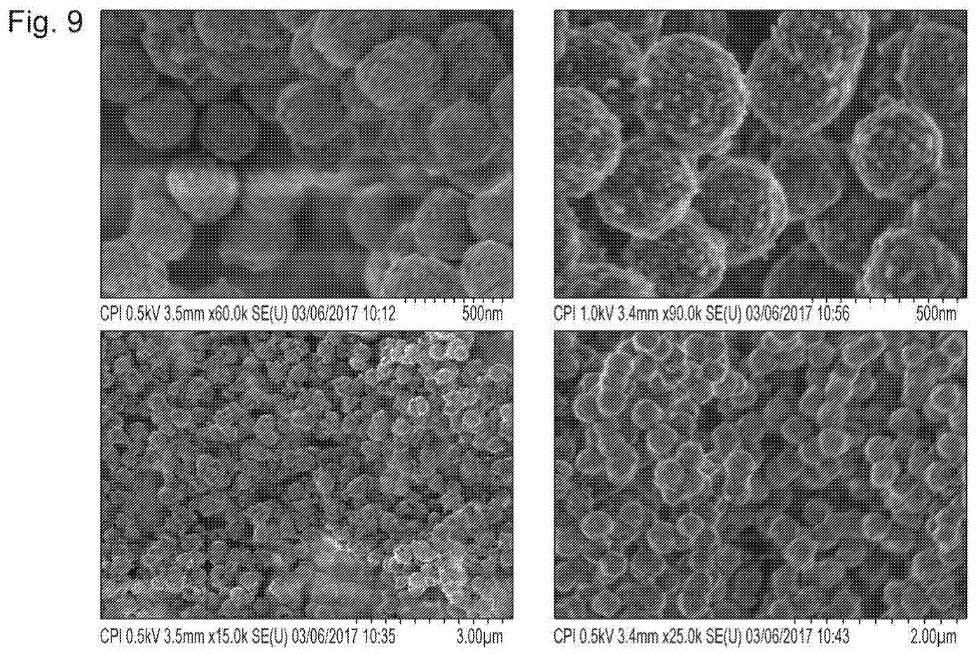

SEM images of the resultant calcined particles are shown in FIG. 9. Lowering of the temperature during shell formation results in a monomodal particle size distribution, with a mean particle size of 336 nm. This result confirms that increasing polymerisation temperature during core foi illation does not adversely affect particle size distribution or surface morphology. However, cooling to 25° C. prior to addition of TEOS assists in eliminating side reactions, particularly formation of solid silica particles. The larger than expected particle size may be attributed to faster polymerisation kinetics during formation of the core. Lowering the polymerisation time during this step should result in reduced particle size. It is also likely the slight overdose of resorcinol will contribute, further increasing particle size.

SiNP005 V2—180 nm Target Particle Size, 35° C. RF Core Polymerisation, 25° C. Shell Formation, 350 rpm Stirrer Speed

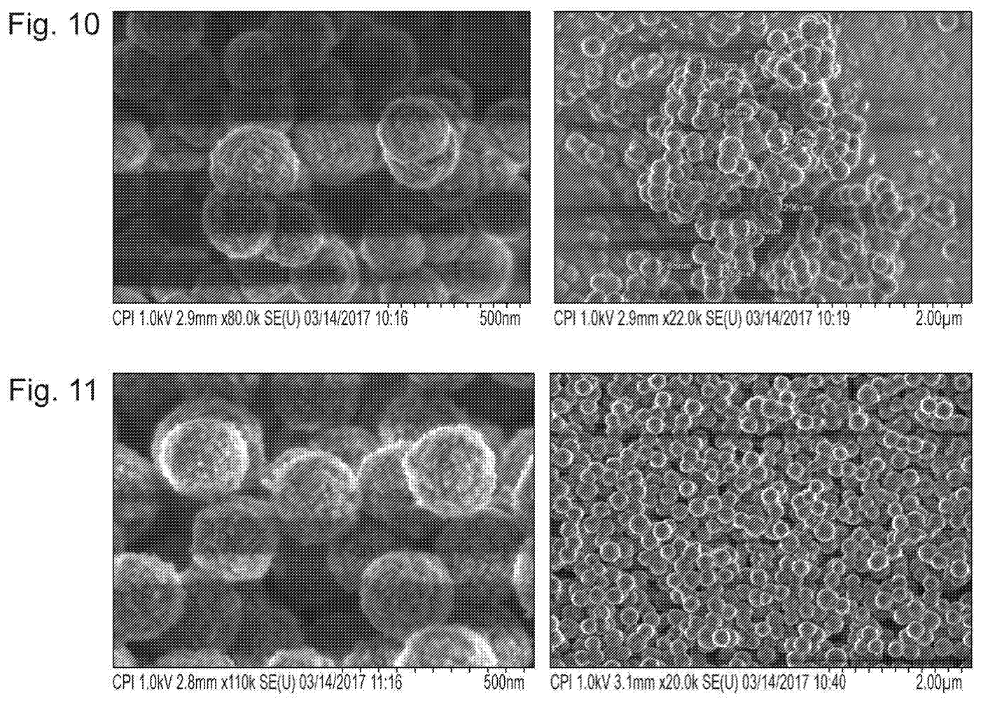

A repeat of the SiNP005 synthesis was carried out using a lower amount of resorcinol to decouple effects of reagent concentration from core polymerisation temperature. SEM images of calcined particles, FIG. 10, show a reduction in mean particle size of ˜80 nm when compared to particles produced during prep SiNP005 (reduction from 336 to 258 nm) suggesting SiNP particle size is sensitive to reagent concentrations during synthesis. Particle size is still larger than the desired 180 nm. However, this is likely attributable to faster polymerisation kinetics in the core and is expected to be adjusted via shortening of core polymerisation time.

SiNP006—180 nm Target Particle Size, 45° C. RF Core Polymerisation, 25° C. Shell Formation, 350 rpm Stirrer Speed

In order to further reduce polymerisation time, polymerisation of the resorcinol formaldehyde core was carried out at 45° C., followed by lowering of the reaction temperature to 25° C. prior to addition of TEOS. Compared to the analogous 35/25° C. prep (SiNP 0005 II), polymerisation occurred at a faster rate, as evidenced by an earlier onset of solution turbidity. SEM images of the resulting calcined particles, without conductive coating, show a mean particle size of 257 nm, FIG. 11. Note that the surface structure of the particles appears to be ‘spikier’ than in previous experiments, which may be result of increased polymerisation temperature. The larger than expected particle size may be explained by the increased reaction temperature; shortening of reaction time during formation of the core may serve to decrease particle size.

SiNP006 II—180 nm Target Particle Size, 45° C. RF Core Polymerisation, 25° C. Shell Formation, 350 rpm Stirrer Speed, Shortened Core Polymerisation Time

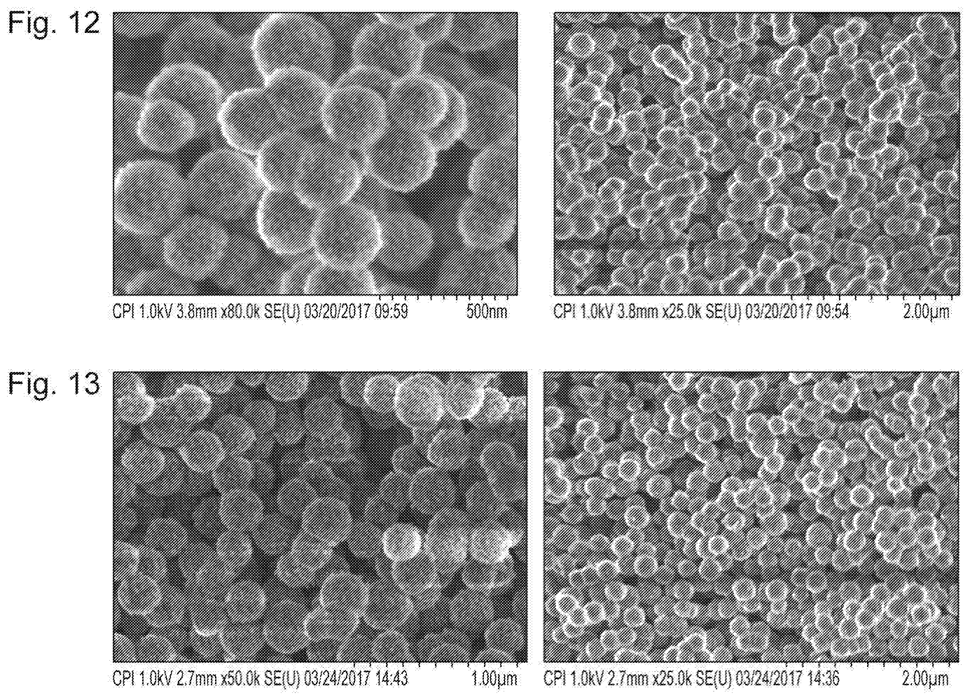

In order to explore the effect of reaction time on particle size a repeat of the SiNP006 prep was carried out in which polymerisation time at 45° C. was reduced to approximately 90 minutes. Consistent with SiNP006 reaction temperature was then lowered to 25° C. prior to addition of TEOS. SEM images of calcined particles, without conductive coating, show a mean particle size of 260 nm, consistent with particles prepared in SiNP006. Surface structure is also consistent with particles prepared in SiNP006, which is ‘spikier’ than in previous experiments, FIG. 12.

SiNP006—180 nm Target Particle Size, 45° C. RF Core Polymerisation, 25° C. Shell Formation, 250 rpm Stirrer Speed, Shortened Core Polymerisation Time

A further repeat of SiNP006 was carried out in which stirrer speed was reduced from 350 rpm to 250 rpm. SEM images of uncoated particles, mean particle size 248 nm, are shown in FIG. 13. Both particle size and surface morphology are consistent with previous SiNP006 syntheses, illustrating that reduced stirrer speed does not result in any significant difference.

SiNP006 IV-180 nm Target Particle Size, 45° C. RF Core Polymerisation, 25° C. Shell Formation, 350 rpm Stirrer Speed, Further Shortened Core Polymerisation Time

An image showing the effect of shortening reaction time for polymerisation of the core from 90 mins to 60 mins can be seen in FIG. 14. The average particle size was determined to be 248 nm, identical to that obtained for SiNP006 III. Particles are larger than target of 180 nm. Subsequent experiment have thus focussed on reducing the monomer concentration during formation of the polymer core in order to evaluate the effect on overall particle size and morphology.

SiNP0007—180 nm Target Particle Size, 45° C. RF Core Polymerisation, 25° C. Shell Formation, 350 rpm Stirrer Speed, Reduced Reagent Concentrations

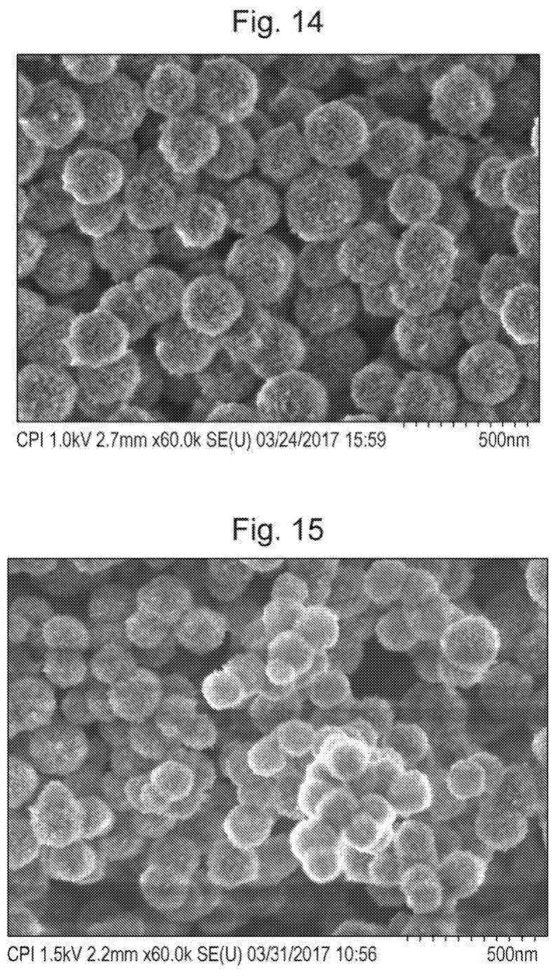

In this experiment the concentration of resorcinol and formaldehyde monomer used in the preparation of the polymer core was reduced by 25%. As seen in FIG. 15 average particle size has been reduced to 188 nm. Also apparent is that this was achieved without adversely affecting the surface morphology of the particles. Careful examination of the image also shows a small amount of particle agglomeration although at this stage it is not known if this is an artefact of the measurement. Further work in which the particles are dispersed into a buffered aqueous solution might be informative to establish if the particles are truly aggregated.

SiNP0007 II—180 nm Target Particle Size, 45° C. RF Core Polymerisation, 25° C. Shell Formation, 350 rpm Stirrer Speed, Reduced Reagent Concentrations, Extended Duration During Cool Down

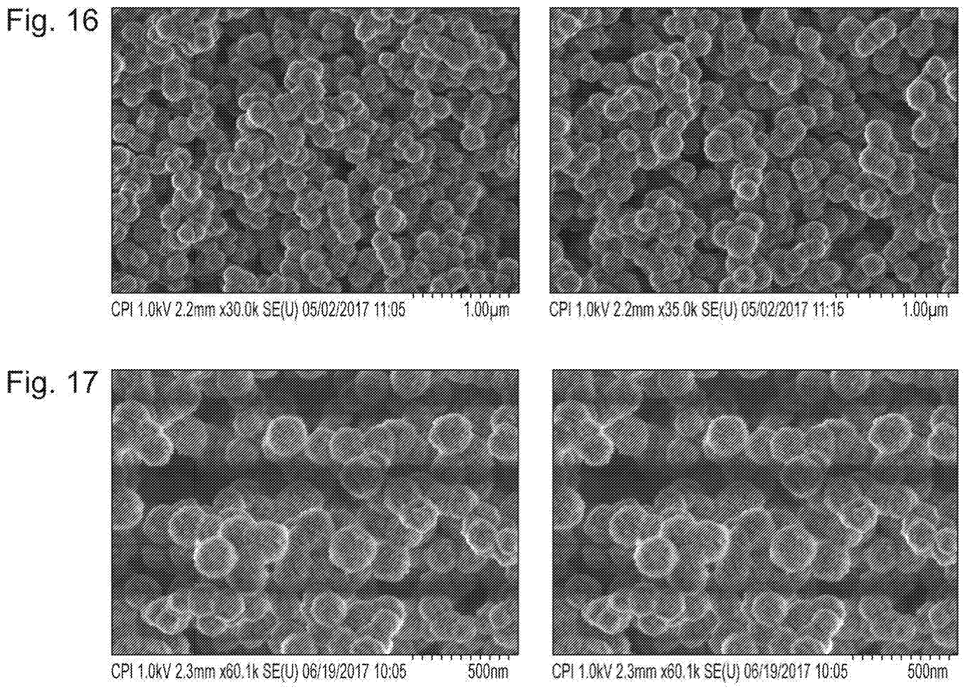

In order to probe the effect of cool down time on particle size and morphology a repeat of SiNP0007 was carried out in which the cool down period was extended by 30 minutes. SEM images of uncoated particles, FIG. 16, show an average particle size of 205 nm. As expected particle size is increased due to the longer reaction time. However, the desired ‘spiky’ surface morphology is still achievable when an extended cool down period is incorporated into the process.

SiNP0007 V—180 nm Target Particle Size, 45° C. RF Core Polymerisation, 25° C. Shell Formation, 350 rpm Stirrer Speed, Reduced Reagent Concentrations

A final prep of SiNP0007 was carried out, resulting in an average particle size of 189 nm, and the correct surface morphology, FIG. 17. It is clear that particles of the correct size and morphology can be prepared at 500 mL scale, and as will be shown in subsequent sections also provide excellent capacity for DNA loading and transfection. Upon scaling, any variations present due to variations in reagent concentration is expected to be much less of an issue and at 5 L scale the process is reproducible and the particles highly consistent.

Characterisation of Nanoparticles

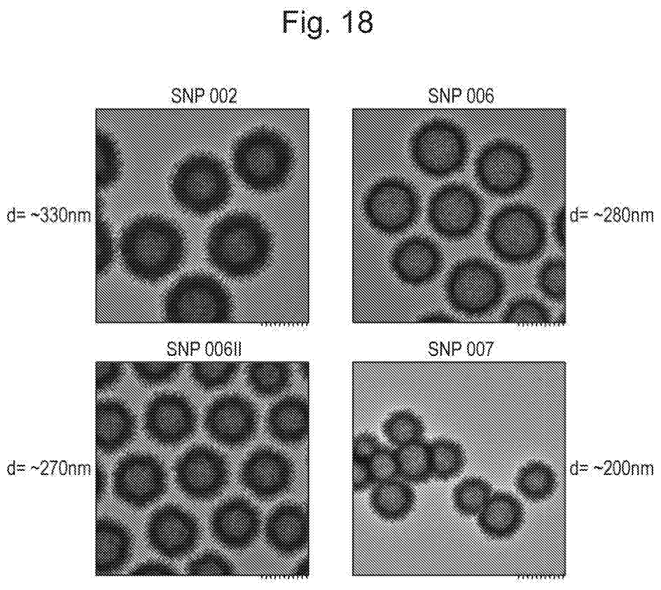

A series of particles were tested via transmission electron microscopy, TEM. Particle size and surface morphology is consistent between SEM analysis carried out, FIG. 18. The particles also show the desired hollow structure.

Based on the characterisation data it can be confirmed that the 180 urn silica nanoparticles prepared by the process of the invention are the correct size and morphology, are fit for purpose and the process should progress to 5 L scale up.

Process Development at 5 L Scale

Following successful demonstration of the synthesis of 180 nm particles with the correct surface morphology and transfection efficacy, the process was scaled to 5 L using a Radley's reactor. A number of syntheses have been carried out to date and these are summarised in Tables 2 and 3. The outcome of each synthesis is discussed in detail in the following sections.

SiNP0008—180 nm Target Particle Size, 45° C. RF Core Polymerisation, 25° C. Shell Formation, 250 rpm Stirrer Speed

In this experiment reaction conditions were maintained as per experiment SiNP0007 with quantities of reagents scale accordingly, Table 2. Due to the increased volume the stirrer speed was reduced from 350 to 250 rpm; based upon results from SiNP006 III a reduction in stirrer speed was shown not to adversely affect particle size or morphology.

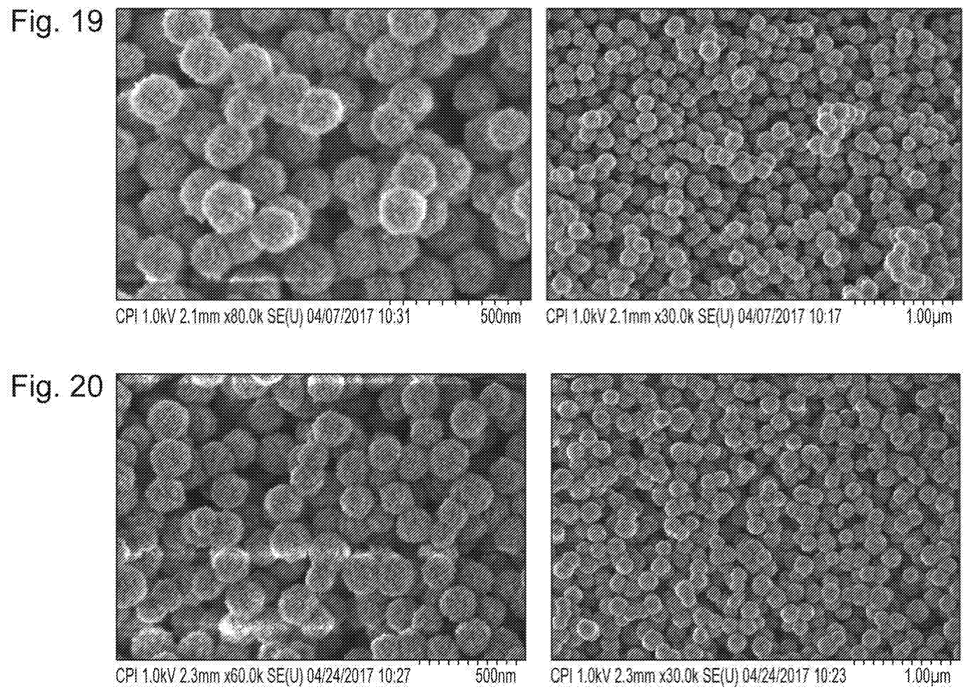

SEM images of uncoated particles are shown in FIG. 19. The particles appear to be highly monodisperse (PDI 0.11), the average particle size is 183 nm, and the surface morphology is exactly as required, illustrating successful scale up.

SiNP0008 II—180 nm Target Particle Size, 45° C. RF Core Polymerisation, 25° C. Shell Formation, 250 rpm Stirrer Speed

In order to check reproducibility experiment SiNP0008 was repeated under identical conditions. SEM imaging of uncoated particles, FIG. 20, shows an average particle size is 184 nm, a monomodal particle distribution (PDI 0.10) and the desired surface morphology, indicating that the process is very reproducible at 5 L scale.

Effect of Calcination Ramp Rate on Morphology

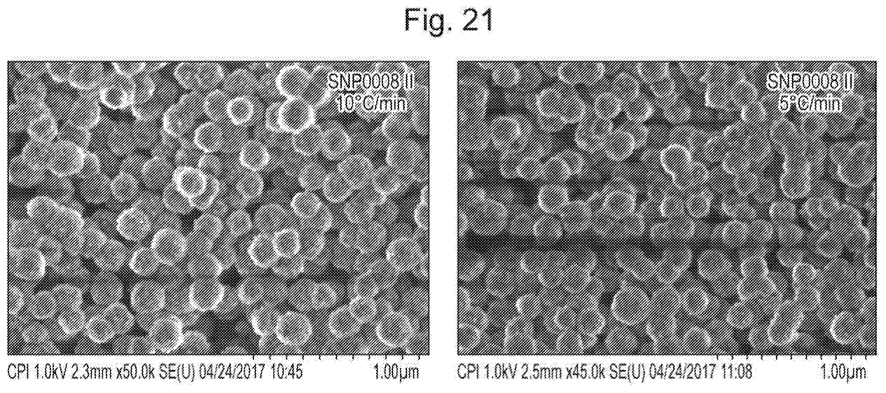

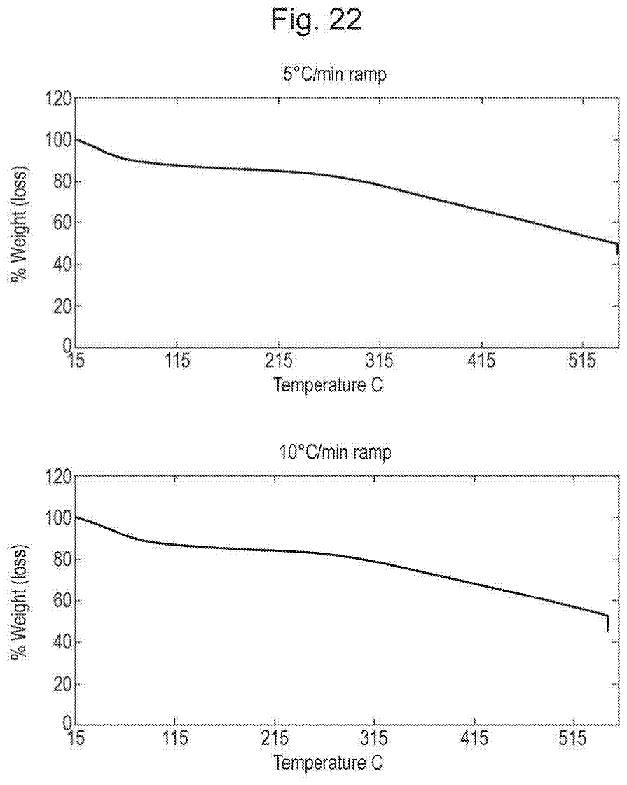

The aim of this experiment was to investigate the effect of different ramp rates in the calcination step on surface morphology in order to potentially shorten the time required for the calcination step. In this experiment SNP 0008 II crude product was used. SEM imaging of uncoated particles, FIG. 21, shows a mean particle size of 183 nm and 186 nm for 5 and 10° C./min, respectively. The silica particles appear ‘spiky’, however compared to SiNP0008 II the “spikes” are less defined and some agglomeration was observed. Thermogravimetric analysis under identical conditions, FIG. 22, shows that the weight loss obtained for SiNP 000811 is similar and not affected by changing ramp rate.

Process Development Targeting Sub 180 nm Particles

Following successful synthesis of 180 nm particles slight modifications to the process were trialled in order to target smaller silica nanoparticles. A number of syntheses have been carried out, the outcome of which is detailed in the following sections.

SiNP0009—130 nm Target Particle Size, 45° C. RF Core Polymerisation, 25° C. Shell Formation, 350 rpm Stirrer Speed

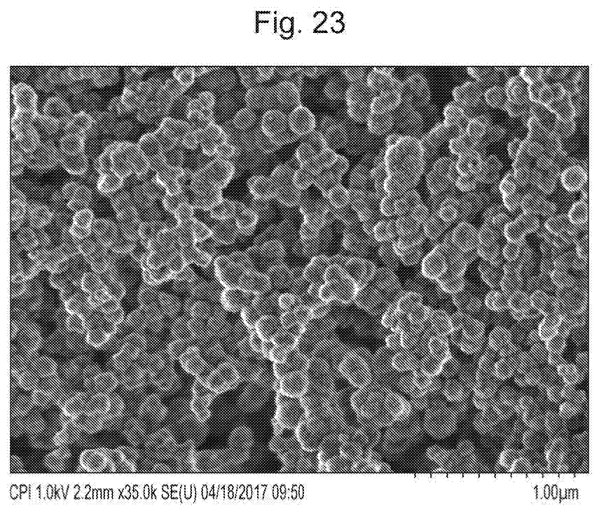

The aim of this experiment was to synthesize 130 nm silica nanoparticles. In this experiment the quantity of resorcinol and formaldehyde was reduced by 52%, all other reaction conditions were maintained as for experiment SiNP 0007. FIG. 23 illustrated unloaded SiNP with an average size of 135 nm. A spiky surface morphology is maintained, although agglomeration is observed. Furthermore, the time required for formation of the polymer core increased from 85 to 175 minutes. This increase in time is due to the reduction in resorcinol and formaldehyde concentrations and hence particle collisions, reducing the rate of the polymerisation reaction.

SiNP0009 II—130 nm Target Particle Size, 45° C. RF Core Polymerisation, 25° C. Shell Formation, 350 rpm Stirrer Speed, Reduced Polymerisation Growth Time

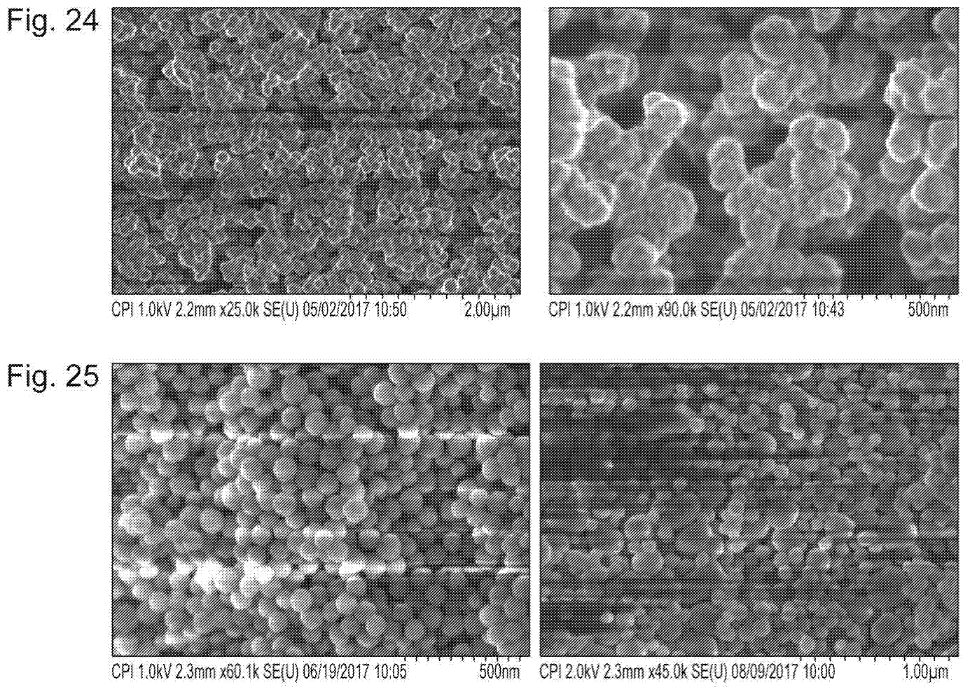

The aim of this experiment was to investigate if polymer reduced growth time is beneficial in the synthesis of 130 nm particles. SEM images of uncoated particles, FIG. 24, show an average particle size of 162 nm with the desired ‘spiky’ surface morphology. The particle size shows a bimodal distribution and agglomeration of particles is observed.

SiNP0009 III—Repeat of SNP0009 (RF Core Formation Only) with Further Reduced Polymer Growth Time

In this experiment the polymerisation growth time was reduced from 200 mins (SiNP 0009 II) to 120 mins at 45° C. and stopped before TEOS addition. Polymerisation occurred at approximately 120 mins. SEM images of the resulting uncoated particles are shown in FIG. 25. The resorcinol formaldehyde particles have an average size of 95 nm.

SiNP 0009 IV 500 mL Radley's Reactor (150 nm Particle Recipe, 45° C. RF Core Polymerisation Only, ˜16% Reduction in R & F)

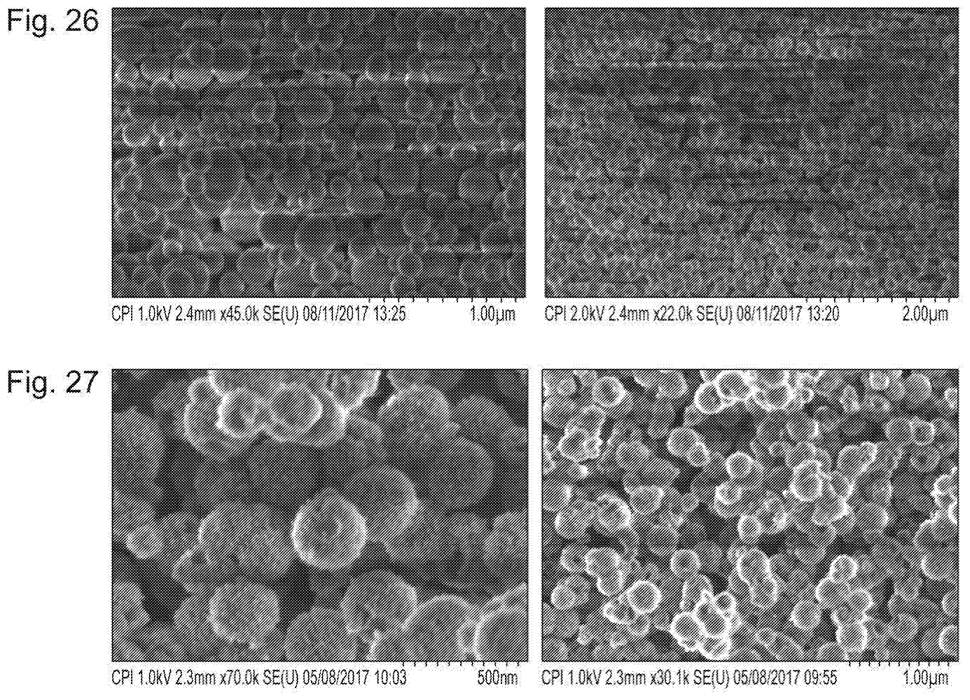

The aim of this experiment was to synthesise nanoparticles of 150 nm size. The quantity of resorcinol and formaldehyde was reduced by 16% compared to SiNP0007, and polymerisation reaction was carried out at 45° C. for 105 mins. Polymerisation, and subsequent precipitation occurred at approximately 125 mins. SEM images of the resulting uncoated RF particles are shown in FIG. 26. The RF particles have an average size of 170 nm.

SiNP0010—180 nm Target Particle Size, 45° C. RF Core Polymerisation, 25° C. Shell Formation, 350 rpm Stirrer Speed

The aim of this experiment was to investigate the role of ammonium hydroxide in this process. In the polymerisation step, no ammonium hydroxide was used (concerns around loss of ammonia at elevated temperature). The key observation from this experiment was that no polymerisation occurred after 195 minutes reaction run time at 45° C. and 350 rpm. With the addition of ammonium hydroxide (14 mL) precipitation occurred as normal after 95 mins.

Reaction temperature was also increased to 45° C. from 25° C. 20 mins after the second addition of resorcinol and formaldehyde. SEM images of the resulting uncoated particles are shown in FIG. 27. The silica particles have an average size of 305 nm, and show a bimodal distribution. Furthermore, holes are observed in some particles. The increased particle size may be a result of the elevated temperature in the 2^{nd} polymerisation step. In addition, this elevated temperature could have weakened the particle structure causing the particle to rupture during the calcination step.

Process Development at 10 L Scale

Following successful demonstration of the synthesis of 180 nm with the correct surface morphology, the process was scaled to 10 L using a Radley's reactor. A number of syntheses have been carried out to date and these are summarised in Table 2 and 3. The outcome of each synthesis is discussed in detail in the following sections. Furthermore, additional safety measures were applied to the process.

Safety Measures Introduced for 10 L Scale Up

A number of safety measures were applied to this process in order to achieve a safe operating envelope at 10 L scale.
1. Nitrogen purging system—eliminate oxygen in the reaction to minimise any chance of ignition.
2. Electrostatic discharge plug—minimise any chance of ignition.
3. Electrostatic discharge additive in the reactor jacket—minimise any chance of ignition.
4. Drager formaldehyde detection—testing for any sign of formaldehyde exposure.

SiNP0011—180 nm Target Particle Size, 45° C. RE Polymerisation, 25° C. Shell Formation, 160 rpm Stirrer Speed

In this experiment reaction conditions were maintained as per experiment SiNP0007 with quantities of reagents scaled accordingly, Table 2. Due to the increased volume the stirrer speed was reduced from 350 to 160 rpm; based upon results from SiNP006 III a reduction in stirrer speed was shown not to adversely affect particle size or morphology.

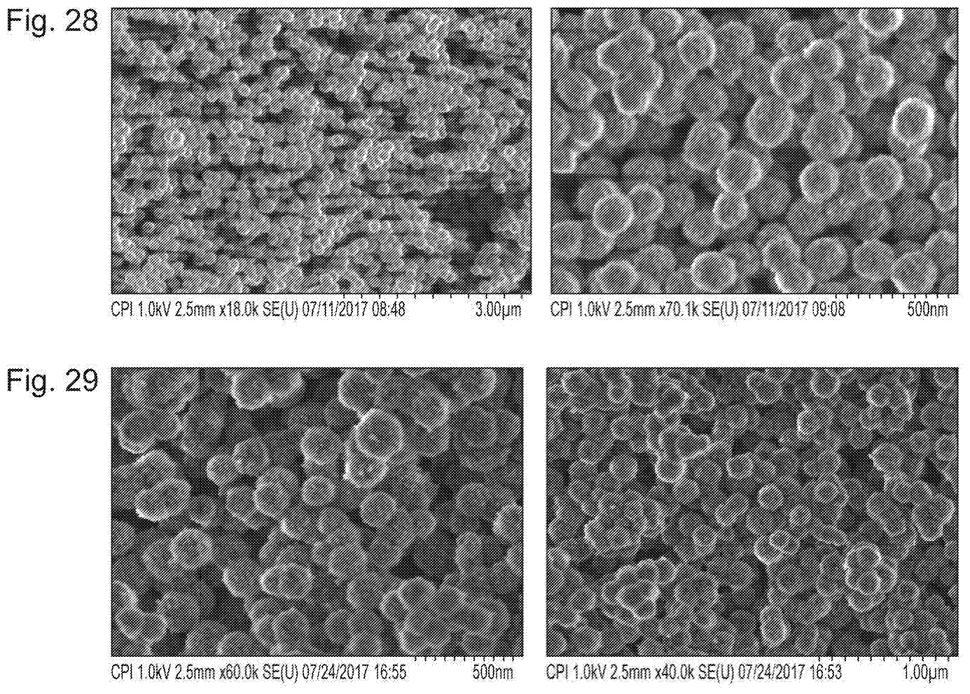

SEM images of uncoated particles are shown in FIG. 28. The particles appear to be highly monodispersed (PDI 0.11), the average particle size is 183 nm, and the surface morphology is exactly as required, illustrating successful scale up.

SiNP0011 II—Repeat of SiNP011

In order to examine reproducibility experiment SiNP0011 was repeated under identical conditions. SEM imaging of uncoated particles, FIG. 29, shows an average particle size of 181 nm (PDI 0.13) and desired surface morphology, indicating that the process is reproducible at 10 L scale. A small percentage of particles were observed have holes, this was due an unforeseen change in ramp rate in the calcination step, however the change was fixed at 298° C.

Analysis of Particle Structure and Morphology Via TEM

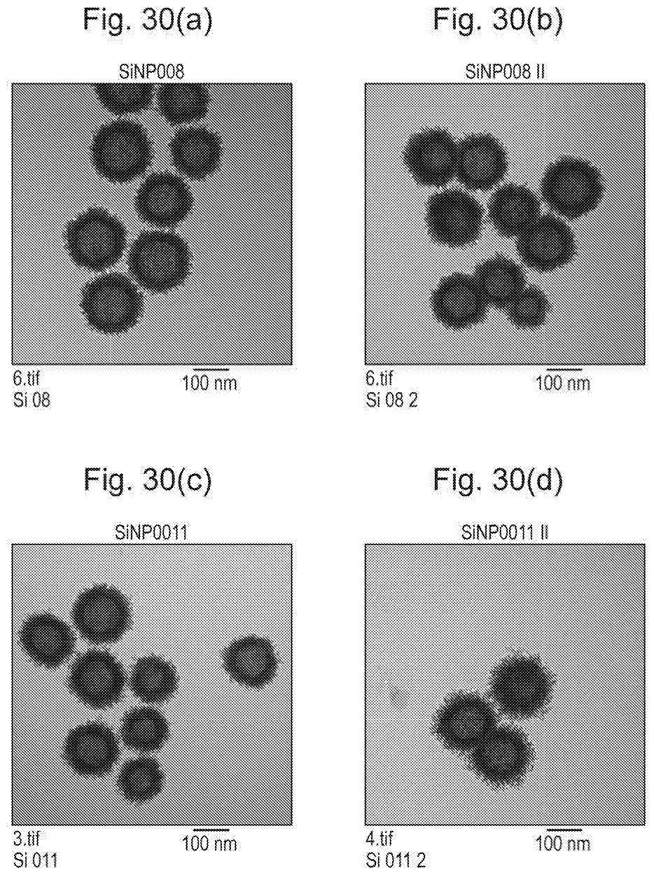

Samples of particles prepared in both 5 and 10 L scale up batches were analysed using TEM to confirm a hollow structure and also surface morphology. Images are shown in FIG. 30. In all cases the particles are hollow, have the desired spiky surface morphology and are ˜180 nm in size, confirming successful scale up of particle synthesis.

Increasing Process Yield

Following successful synthesis of 180 nm particles slight modifications to the process were trialled in order to increase the quantity of silica nanoparticle per volume of solvent. A number of syntheses have been carried out, the outcome of which is detailed in the following sections.

SiNP0012—500 mL Radley's Reactor (180 nm Particle Target Size, 45° C. for 90 mins, Stop Before TEOS Addition, 5 Times Concentration of R & F)

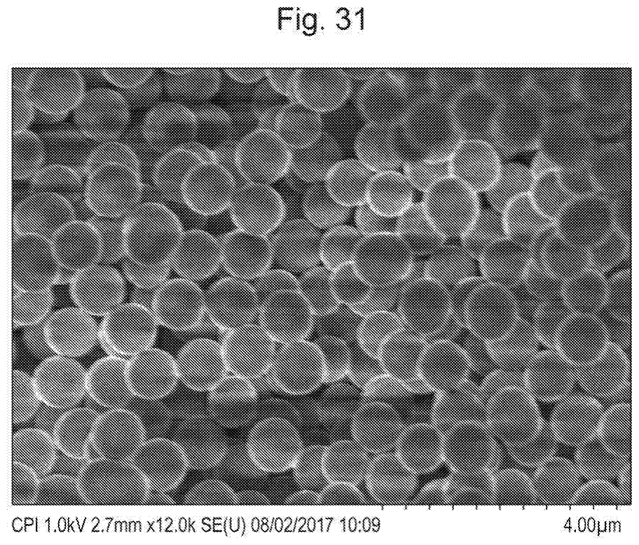

The aim of this experiment was to synthesise 180 nm silica nanoparticles at a higher concentration of reagents in solution. In this experiment the quantity of resorcinol and formaldehyde was increased by 5 times relative to experiment SiNP0007. The reaction was carried out at 45° C. for 90 mins and cooled to 25° C. before stopping the experiment. FIG. 31 illustrates unloaded SiNP with an average size of 890 nm. During the experiment polymerisation occurred in approximately 23 mins. The increased rate of polymerisation and increase of particle size was expected, due to the increase concentration of resorcinol and formaldehyde. Formation of larger particles, rather than a greater number of particles, suggests that the concentration of reagents is at supersaturation.

SiNP0012 II—500 mL Radley's Reactor (180 nm Particle Target Size, 10° C., Stop Before TEOS Addition, 5 Times Concentration of R & F) (Reference Example)

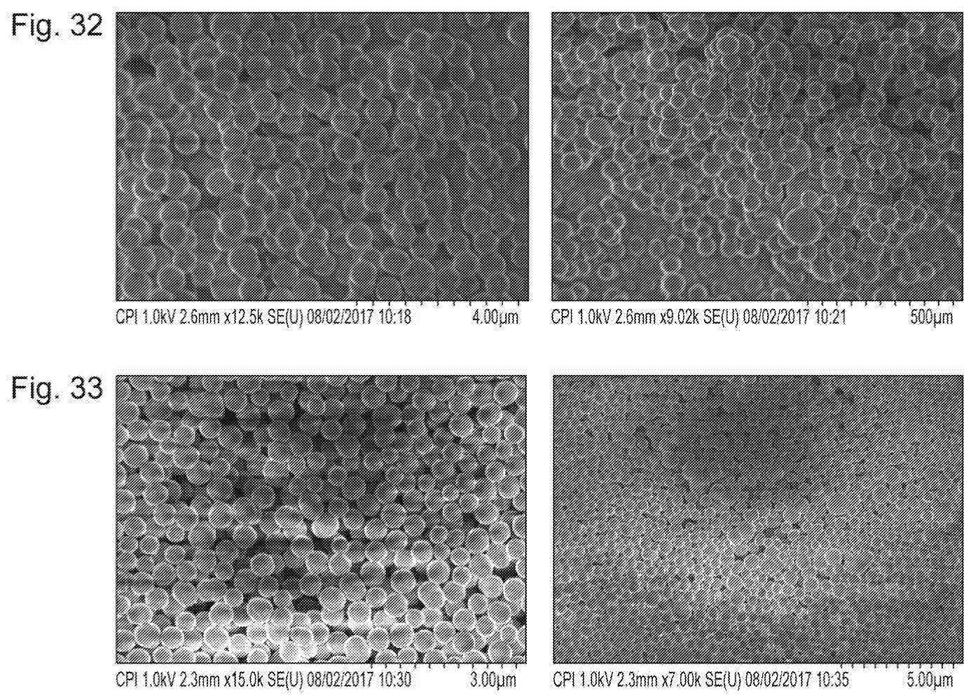

In this experiment the reaction conditions were similar to SiNP0012, quantity of resorcinol and formaldehyde remained unchanged, reaction stopped before TEOS addition however the reaction was carried out at 10° C. FIG. 32 illustrates unloaded SiNP with an average size of 644 nm. At 10° C. no polymerisation reaction occurred after 120 mins from the initial start, temperature was increased to 25° C. (11 mins), polymerisation occurred after 45 mins.

SiNP0012 III—Repeat of SiNP0012 with Reduced Polymerisation Time

The reaction was carried out at 45° C. for 7 mins and cooled down to 25° C. before stopping the reaction. Polymerisation occurred 23 mins into the cool down stage. FIG. 33 illustrates uncoated RF particles with an average size of 412 nm. Compared to SiNP0012, the reduce polymerisation time did result in reduction of particle size, however it is not possible to achieve an average particle size of 180 nm without higher cooling power.

SiNP0012 IV—500 mL Radley's Reactor, (180 nm Target Particle Size, Stop Before TEOS Addition, ˜24% Reduction in R & F)

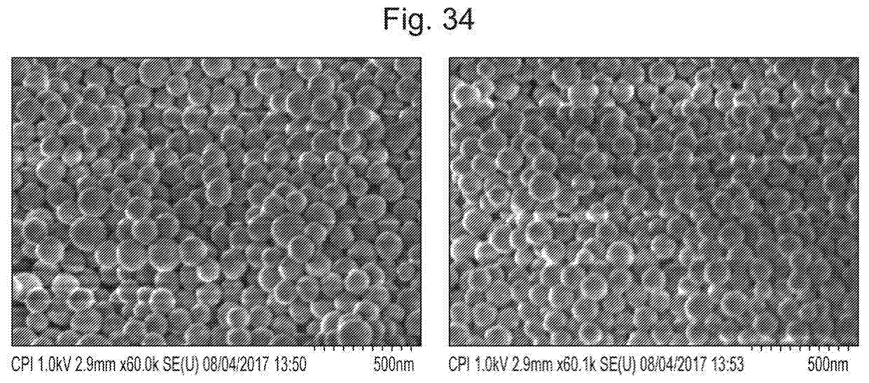

In order to confirm RF core size for a known synthetic procedure the SiNP0007 batch was repeated however the reaction was stopped prior to TEOS addition. FIG. 34 illustrates uncoated RF particles with an average size of 128 run, consistent with the core sizer observed for 80 nm core/shell spiky particles.

Comparison of Particle Size for SiNP Prepared

Table 4 shows the particle size obtained for each of the silica nanoparticle preps carried out at CPI. Most of the early samples show a mean particle size in the region of 250 nm which would indicate that reaction time falls within the plateau region in size development of the polymer core. Reducing monomer concentration leads to a corresponding reduction in the size of the core, as illustrated in SiNP0007 (500 mL scale), SiNP0008 (5 L scale) and SiNP011 (10 L scale).

TABLE 4
Mean particle size (uncoated particles) measured using SEM

| Experiment | Mean particle size (nm) |
|---|---|
| SiNP 0001 | 242 |
| SiNP 0002 | 300 |
| SiNP 0003 | 168 |
| SiNP 0004 | 367 |
| SiNP 0005 | 336 |
| SiNP 0005 II | 258 |
| SiNP 0006 | 249 |
| SiNP 0006 II | 263 |
| SiNP 0006 III | 248 |
| SiNP 0006 IV | 248 |
| SiNP 0007 | 188 |
| SiNP 0007 II | 205 |
| SiNP 0007 III | 205 |
| SiNP 0007 IV | 248 |
| SiNP 0007 V | 189 |
| SiNP 0008 | 183 |
| SiNP 0008 II | 184 |
| SiNP 0009 | 135 |
| SiNP 0009 II | 162 |
| SiNP 0009 III | NA |
| SiNP 0009 IV | 95 |
| SiNP 0010 | 305 |
| SiNP 0011 | 178 |
| SiNP 0011 II | 181 |
| S1NP 0012 | 890 |
| S1NP 0012 II | 644 |
| SiNP 0012 III | 412 |
| S1NP 0012 IV | 128 |

Conclusions

Particles having an average size of ˜300 nm were produced on a 100 mL scale. Work then focussed on scaling (500 mL, 5 L and 10 L) and process improvement for synthesis of 180 nm SiNP using a Radley's reactor for precise control of process parameters Significant progress was made in reducing the process time for formation of the resorcinol formaldehyde core. In addition it is now understood that polymerisation temperature may be increased during formation of the core without detrimental effect upon particle surface structure, in fact this is generally beneficial. Formation of the silica shell is preferably conducted at 25° C. to avoid formation of a bimodal particle distribution, likely to contain solid silica nanospheres in addition to the desired hollow, spiky particles.

The process has been successfully scaled to 500 mL, 5 L and subsequently 10 L, resulting in 180 nm particles with low polydispersity, the correct surface morphology, as evidenced by SEM and TEM, and porosity. Control of particle size improves significantly as the process is scaled. An increase in the concentration of particles obtained per litre of reaction solvent is also observed, increasing to a maximum of 1.7 g/litre at 10 L scale. Based on the characterisation data it can be confirmed that the 180 nm silica nanoparticles prepared at 10 L L scale are the correct size and morphology and are fit for purpose. Loading of SiNP produced using the modified synthetic process with polyethyleneimine is considered in Example 2.

### Example 2—Loading of SiNPs with PEI

Materials and Methods

Materials

The materials used within silica nanoparticle modification are given in Table 5.

TABLE 5
Materials used

| Reagent | Details |
|---|---|
| Silica Nanoparticles | CPI |
| Water | De-ionised |
| 1,3-(trihydroxysilylpropylmethylphosphonate | Sigma Aldrich, |
| monosodium salt) [THPMP] | Product Code |
| | 435716 |
| Polyethyleneimine, 10,000 MW, Branched | Alfa Aesar, |
| | Product Code |
| | 40331 |
| Sodium Carbonate | / |
| Sodium Bicarbonate | / |

Methods

PEI loading of silica nanoparticles produced in Example 1 was carried out. This includes 2 steps, the first is phosphonate linking which consists of mixing a phosphonate linker, the 3-(Trihdroxysilyl) propylmethyl phosphonate monosodium salt solution (THPMP) with the Silica Nanoparticles (SNP) for 2 hours at 40° C. The second step is the Polyethylenimine (PEI) loading i.e. mixing of phosphonate linked silica Nanoparticles with PEI, which is present at 5 times excess compared to silica. This process takes place over 4 hours at room temperature.

As described above the process is lengthy taking in excess of 4 hours to complete, a series of experiments were performed to improve the process by looking to reduce the mixing time of each step and also to examine the effect of increasing the temperature and following the reaction at different times for each step. The changes have been analysed in two different ways: Zeta potential and Carbon Hydrogen Nitrogen (CHN) analysis.

PEI Loading—Lab Scale

The PEI loading was carried out on nanoparticle batches SNP008, SNP008-II, SNP007, SNP007-VI,

SNP011, and SNP011-II produced in Example 1. Due to the low amount of product obtained to run CHN analysis a scale up of this process was done using 200 to 300 mg of silica instead. All the ratios between components have been kept at the same level.

PEI Loading—250 ml Scale and 100 mg of SNP008

Reactions were carried out in a 250 ml Radley's reactor equipped with an angled 4-bladed propeller and data logging capability for temperature, pH and stirrer speed. The quantity of SNP introduced was increased from 30 mg to 100 mg. The quantity of the other reactants was increased to maintain the same ratio of reactants. The volume of solvent used was adjusted to fit the reactor i.e. 100 mL of H₂O was used to dissolve and disperse the THPMP and the SNP. Moreover 100 mL and 50 mL of carbonate buffer (pH 9.8) were used to suspend respectively the PEI and the phosphonate linked SNP. A yield of 50% (53 mg) was obtained. The materials used are given in Table 6 below.

TABLE 6
Materials used

| Reactant | Quantity | |
|---|---|---|
| SNP | 100 | mg |
| THPMP | 710 | μL |
| PEI-10K | 500 | mg |

Study of the Phosphonate Linking—500 ml Scale, 500 mg of SNP008

Reactions were carried out in a 500 ml Radley's reactor equipped with an angled 4-bladed propeller and data logging capability for temperature, pH and stirrer speed. The focus was on the optimisation the reaction time of the phosphonate linking step. After the mixing of SNP and THPMP the particles were first centrifuged at 10 000 rpm for 10 minutes then the supernatant was removed and the particles were re-suspended in H₂O and centrifuged again using the same conditions. Finally the supernatant was removed again and the particles were dried at room temperature for two days.

During this adsorption study samples of 40 mL were taken every 30 minutes. The experiments were carried out at 3 different temperatures (40° C., 50° C., 60° C.) to explore the influence of the temperature on the reaction's speed.

The SNP amount was increased from 30 mg to 500 mg however the ratio between reactant remained the same. This was done to ensure that a sufficient amount of product was obtained in each sample.

The volume of solvent used was adjusted to fit the reactor i.e. 220 mL of H₂O was used to disperse the SNP and dissolve the THPMP. Materials used are given in Table 7 below.

TABLE 7
Materials used

| Reactant | Quantity | |
|---|---|---|
| SNP | 500 | mg |
| THPMP | 3.550 | mL |

Study of the PEI Loading—500 mL Scale, 500 mg of SNP008

Reactions were carried out in 500 ml Radley's reactor equipped with an angled 4-bladed propeller and data logging capability for temperature, pH and stirrer speed. The focus was on the optimisation of the reaction time during the PEI loading step. The decision to prepare first a solution of phosphonate linked particles was made. Consequently for the different experiments that follow the PEI was loaded from the same preparation solution.

The conditions of this solution were as follows:

TABLE 8
Materials used

| Reactant | Quantity | |
|---|---|---|
| SNP | 1500 | mg |
| THPMP | 10.650 | mL |

| | |
|---|---|
| H₂O | 220 mL × 2 |

After the mixing and stirring at 40° C. for 2 hours followed by centrifugation and washing, the particles were re-suspended in 480 mL of Carbonate buffer solution. A third of this solution (160 mL) was then used for the different experiments. In parallel to this step, 2.5 g of PEI-10K was suspended in 320 mL of Carbonate buffer solution.

During this study the experiments were carried out at 2 different temperatures 30° C. and 50° C. Samples were taken after 45 min, 1 h 45 min, 2 h 45 min, and 4 h for each temperature. The volume of sample collected at 30° C. was 40 mL and 80 mL at 50° C. This decision to double the volume collected was made because of the small amount of product obtained (˜15 mg) after the 30° C. experiment.

Final Scaled Up Modification Process

After completing several experiments a final scaled up modification process was adopted that gave good adsorption as indicated by nitrogen content. All of the quantities have been multiplied by 10. Phosphonate linking reactions were carried out at 40° C. for 2 hours in a 250 ml Radley's reactor equipped with an angled 4-bladed propeller and data logging capability for temperature, pH and stirrer speed. PEI loading was done inside glass bottles on hotplate stirrers at room temperature.

The conditions used were as follows:

TABLE 9
Materials used

| Reactant | Quantity | |
|---|---|---|
| SNP | 300 | mg |
| THPMP | 2.150 | ml |
| H₂O | 100 × 2 | |
| Carbonate buffer pH 9.8 | 100 + 50 | |
| PEI | 1.5 | g |

Analysis of the Surface Charge Using Dynamic Light Scattering Zeta Potential

Dynamic Light Scattering was used to characterise the surface charge and particularly the isoelectric point (IEP) of the particles. The Zeta potential as a function of pH was measured using a Horiba SZ-100 Nanoparticle analyser. Using this technique gives information about the surface charge of the particles. IEP is achieved when the Zeta potential reaches 0 mV. Knowing the IEP of unmodified and PEI saturated particles enable us to follow the evolution of the PEI Loading. When adsorption begins on a bare particle surface the IEP will move towards that of a fully saturated surface. Furthermore it should be noted that this techniques is quite inaccurate for pH<2 and pH >12.

The samples were prepared by dispersing the solid particles in acidic or basic solutions. This solutions were prepared by adding HCl or NaOH (10⁻²M) dropwise in 100 mL 10⁻³M KCL solution. The particles were dispersed in acid medium when a high IEP was expected and vice versa. Then drops of acid or base were added to change the pH of the solutions and monitor the evolution of the surface charge of the particles.

Analysis of the PEI Concentration Using the C:H:N Analysis

C:H:N analysis allowed us to check if PEI has been successfully loaded on to the particle and to quantify the amount adsorbed. The technique measures the percentage of Carbon, Nitrogen and Hydrogen on the particle surface. For the purpose of this study the focus of the analysis was on Nitrogen content, a major component of PEI. By analysing different samples at different times and different temperatures it provided information on the understanding of the reaction and its speed. This is a complementary technique to the Zeta potential analysis and allows correlation of both sets of results. A minimum of 30 mg of sample is required to run a single analysis.

Scanning Electronic Microscopy

Scanning electronic microscopy was used to check if phosphonate linking or PEI loading had induced any change on the morphology of the particles.

Results and Discussion

PEI Loading on SNP008

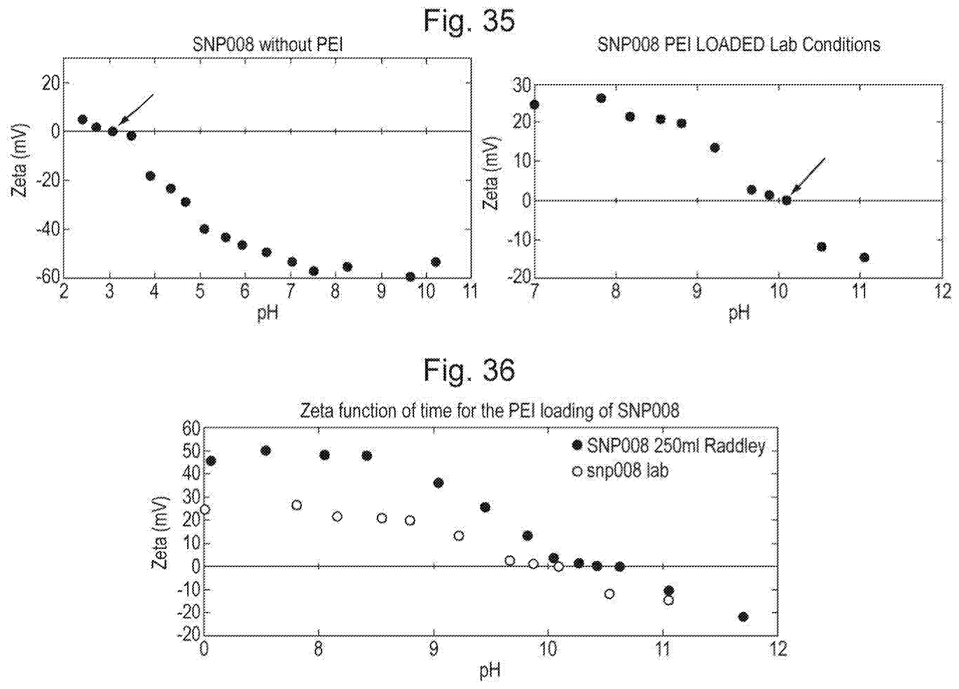

Zeta potential analysis was carried out on unmodified and modified silica nanoparticles. FIG. 35 shows results from zeta potential analysis using DLS. A huge difference in IEP is observed. The IEP of uncoated SNP008 is around pH3 whereas when PEI is loaded IEP increases to pH 10. This is consistent as it is expected that the silica surface is negatively charged without coating and positively charged once PEI is loaded. These results are expected as particles without coating contain hydroxy groups on their surface whilst PEI loaded particles contain dimethylamine groups, which have a pKa of around 10.5.

SNP 008—250 mL Scale—100 mg SNP

A first scale up was carried out by increasing the amount of particles to 100 mg (vs 30 mg) in 250 ml Radley reactors. FIG. 36 displays the results from zeta potential analysis of scaled up particles. A minor difference in the IEP is observed but due to the accuracy of the equipment the difference is not significant. However for lower pH when a plateau is reached we noticed that there is a difference of approximately 20 mV in the magnitude between the 2 samples. In general, the higher the magnitude of charge the more stable the particles are.

Phosphonate Linked SNP008

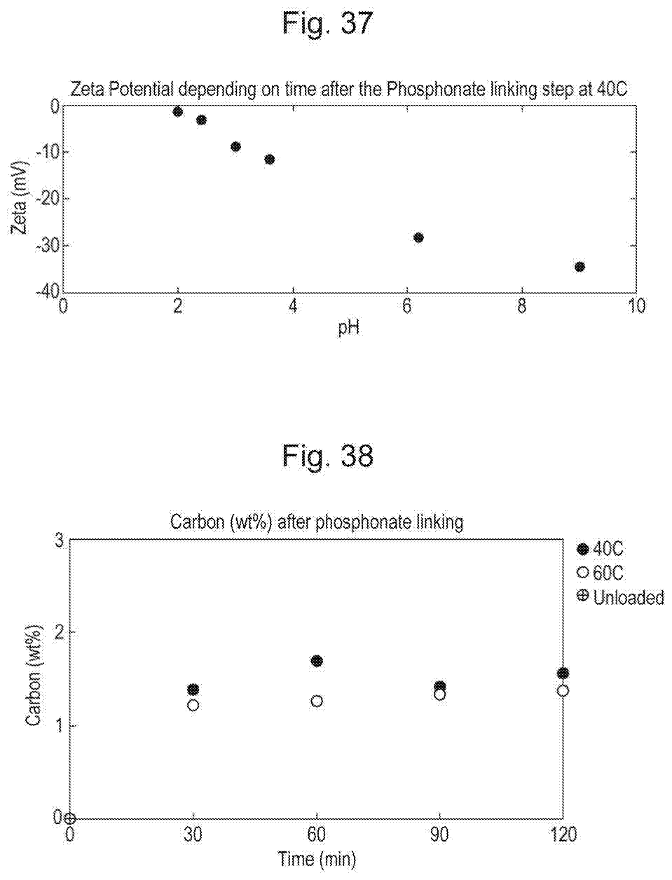

A series of experiment related to the optimization of the phosphonate linking step were carried out to establish if it was possible to stop the modification process earlier. FIG. 37 describes the evolution of Zeta potential as a function of pH for SNP obtained as described above.

As observed for unmodified SNP, the surface is negatively charged having an IEP of approximately pH3, but following treatment a slight decrease in IEP to less than pH2 is observed. Results below pH2 are inaccurate but it is assumed that IEP of these particles tends towards the pKa of phosphorous acid which is around 1.5. This result confirms that particles are more negatively charged during the PEI loading. Monitoring the phosphonate linking by collecting samples at different times to optimise the process is not possible using the Zeta potential analysis as the IEP is below the pH limits of the machine.

An attempt to optimize this step was done later, with the resulting particles analysed using C:H:N analysis. The phosphonate linker source contains propyl and methyl groups and it was thought that carbon content at surface could be a good indicator to monitor this step. However, the results obtained were quite random between batches indicating that contamination from the environmental was likely (some samples were observed with both a low C and a high N content).

However inside a same batch the carbon content is quite constant with time as displayed in FIG. 38. Through this result we think that the mixing time during this step can be dramatically reduced since after only 30 minutes of mixing (1^{st} point of measurement) a plateau is reached.

Optimisation of the PEI Loading Step—SNP008—500 mL Scale, 500 mg SNP, 30° C.

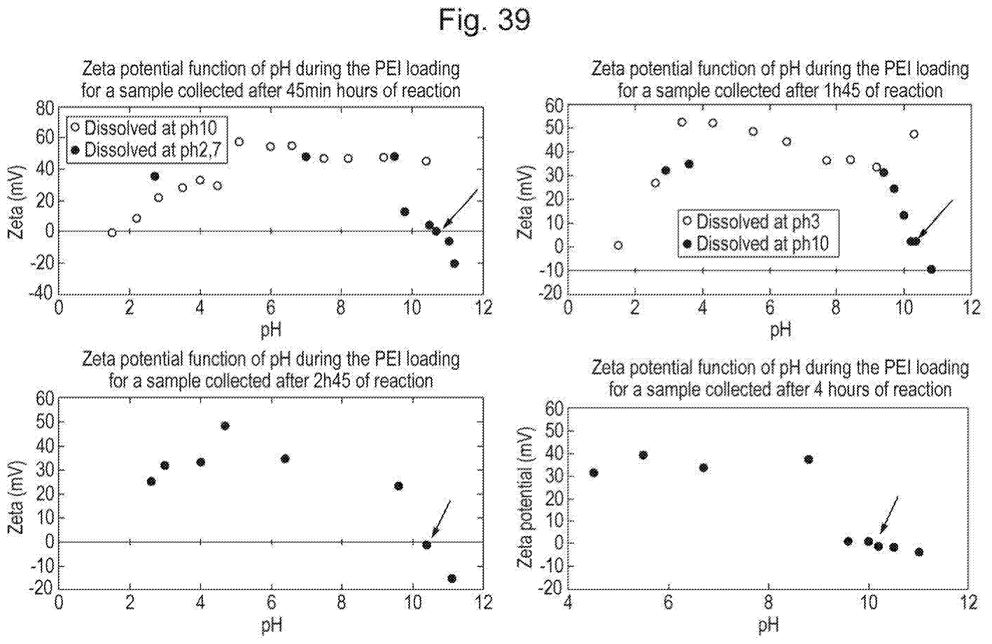

The graph above describes the evolution of the Zeta potential as a function of pH for SNP obtained as described above. FIG. 39 also describes the evolution of zeta potential as a function of pH at different times of the reaction. These experiments were carried out in order to reduce the initial 4 hours of PEI loading mixing step. Most of the samples were prepared by dissolving 2 mg of particles in 100 mL of acid solution. By doing this, it was expected to get a shift in the IEP from approximately 2 (phosphonate linked particles) to 10 (fully coated particles), but also probably reach a plateau which means that all the surface was saturated by PEI. However barely 45 minutes (i.e. the first measured point) after the reaction starts the plateau was reached as is shown in FIG. 38. On the basis of these results the hypothesis that the maximum PEI loading capacity of the particles was reached after 45 minutes of reaction therefore it is not necessary to run the reaction for 4 hours. This can be explained by the fact that PEI is introduced 5 times in excess compared to silica particles. Considering this, further experiment can be carried out to reduce the amount of PEI introduced therefore cut costs.

A second observation was made relating to the dispersion of the particles during the analysis. The two charts on the top of the FIG. 39 display the results when particles were dispersed in an acid and basic medium. Although not fully understood, it may be that the curve, starting as expected with a high positive charge for a coated surface at pH10, might be showing destabilisation of the PEI as the medium becomes more acidic.

Evaluation of the PEI Loading Rate

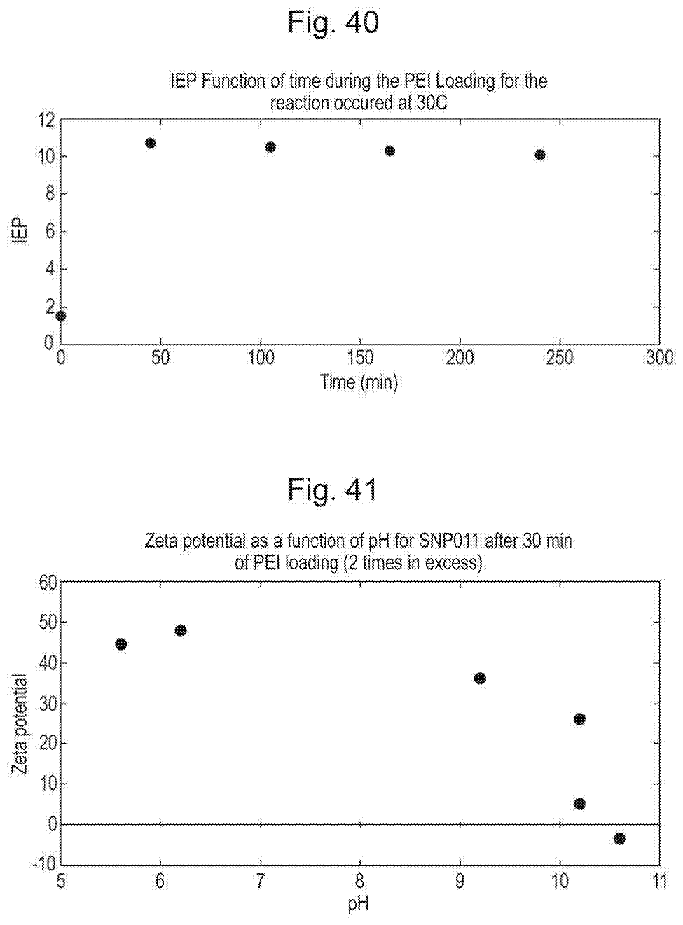

As shown in FIG. 40, the IEP reaches a plateau for the first point of measurement (45 min). On the basis of these results the hypothesis has been made that full loading capacity was reached after 45 minutes of reaction and it is not necessary to run the reaction for 4 hours. It is also thought that the quantity of PEI can be reduced as it is added in excess although an optimisation would have to be performed.

The following set of experiments were carried out to determine if the amount of PEI introduced can be reduced and also examine the rate of loading to the particle surface. To do that, experiments on SNP011 and SNP011-2 were carried out using 2.5 times lower amount of PEI than usual. The conditions were as shown in Table 10.

TABLE 10
Experimental conditions

| PEI loading | | | | Mass | Mass |
|---|---|---|---|---|---|
| Time | | Type | Equipment used | SNP | PEI |
| 5 | min | SNP011_II | Hotplate stirrer | 200 mg | 400 mg |
| 10 | min | SNP011_II | Hotplate stirrer | 200 mg | 400 mg |
| 15 | min | SNP011_II | Hotplate stirrer | 200 mg | 400 mg |
| 30 | min | SNP011 | Hotplate stirrer | 200 mg | 400 mg |
| 1 | h | SNP011 | Hotplate stirrer | 200 mg | 400 mg |
| 1 | h 30 | SNP011 | Hotplate stirrer | 200 mg | 400 mg |
| 2 | h | SNP011 | Hotplate stirrer | 200 mg | 400 mg |

First a zeta potential analysis was run after 30 min of reaction in order to check if an eventual s the IEP between 2 to 10 can be seen. The result is displayed below in FIG. 41.

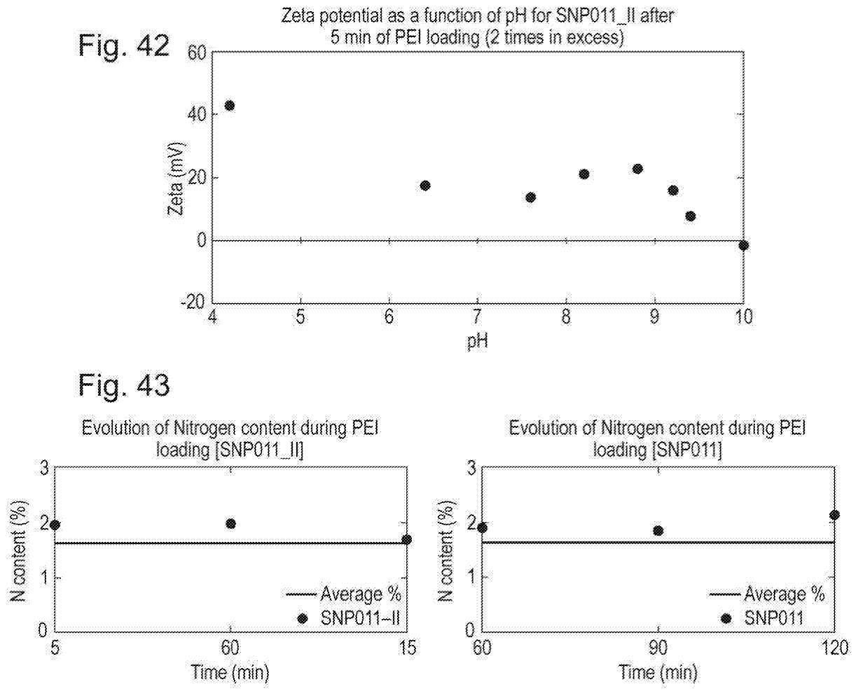

In FIG. 41, the IEP is around pH10.5. This value is quite similar to the IEP previously found although in this case the PEI was introduced in ×2 times excess (vs 5 times before) and the adsorption reaction was carried out for only 30 minutes. In order to have a better estimation of the loading rate, one more analysis was then run after only 5 minutes of PEI loading (2 times in excess), FIG. 42.

After 5 minutes of reaction the IEP is still quite similar to before. However the magnitude of the plateau is dramatically decreased as it was around 40 mV in FIG. 41 and it is just around 20 mV here.

It is assumed that PEI covers the particle surface very quickly and that is why surface of the particles is positively charged after just 5 minutes of reaction. Nevertheless a huge difference is observed for the overall zeta potential at pH <9 between FIG. 41 and FIG. 42. It is assumed that this difference is due to the lower amount of PEI loaded after 5 minutes of reaction but it can also come from the use of different sample type for each experiment. Moreover it was noticed that for results displayed in FIG. 41 (2.5 times lower amount of PEI than usual and only 30 min of reaction) the charge magnitude level and the IEP are the same as the particles modified with the usual conditions (PEI 5 times in excess—4 hours). In order to confirm these observations, C:H:N analysis was carried out on the set of experiments listed in Table 10. The results are shown in FIG. 43 below.

FIG. 43 shows the evolution of nitrogen content during PEI loading on two different batches of particles (10 L batches) and two different time scales. An average nitrogen content is depicted on both graphs.

The results shown in FIG. 43 were obtained using PEI 2 times in excess (vs 5 times UQ's process), which means 60% less polymer than usual. Firstly the overall Nitrogen content is constant for both batches with time and it is around 2% which is higher than the average content observed using the usual process. This higher value is explained in the next sections by the fact that here, phosphonate linking has been done in Radley reactors which have much better temperature control. Secondly it is observed that after just 5 minutes of mixing the value of 2% nitrogen is reached and it stays at the same level after. This is an interesting result as it may indicate that (i) the amount of PEI can be reduced dramatically and (ii) the initial 4 hours of mixing can be reduced to 5 minutes. However, in this instance, the transition between the end of the mixing and the centrifugation step that followed was not particularly controlled, hence the loading process could have continued by diffusion after mixing had finished. An additional experiment, in which transfer to the centrifuge was carried out immediately post mixing was carried out to confirm the loading level. A nitrogen content in the same range (1.82%) was obtained, illustrating that a 5 minute mix is sufficient for PEI loading to occur.

PEI Loading on SNP07/07_VI/08/08_II/011/011_II

TABLE 11
Nitrogen content (wt %) on Lab scaled particles

| | | N (wt %) | |
|---|---|---|---|
| | N (wt %) | Second | BET_{surface} |
| Sample | First process | process | (m²/g) |
| SNP-07-UQ | / | 1.64 | / |
| SNP-07-VI-UQ | 1.36 | 1.55 | 71 |
| SNP08-UQ | 1.56 | 1.68 | 121 |
| SNP08-II-UQ | 1.88 | 1.93 | 156 |
| SNP011 UQ | / | 1.76 | 113 |
| SNPO11-II-UQ | 2.37 | 1.23 | 159 |

In order to check that PEI was correctly loaded on the particles synthetized previously, C:H:N analysis was carried out. The results are shown in Table 11 above. PEI loading was carried out by a first process. Due to the low amount of product collected to run C:H:N analysis for some of the samples, PEI loading was then carried out by using a quantity increased variation the first process (second process). Reactions were done in glass bottles using a hotplate stirrer. The overall nitrogen content of these PEI loaded particles are lower than the expected level of 2.5-3.4% observed by University of Queensland. Moreover a slight difference between particles is observed. In order to understand the origin of the difference BET surface analysis were carried out on the different unloaded particles. The results in Table 7 show that 08_11 and 011_11 have the higher surface area and

PEI content. Furthermore, as mentioned before 250 ml glass bottles and hotplate stirrer were used for the modification of some of the samples (second process). It is assumed that the heating inside the solution was not well distributed and that explain anomalies like 1.23% nitrogen content observed on SNP011_2 for the second process and also an overall low nitrogen content.

Increase of PEI Loading Level

Following the results of the C:H:N analysis displayed in Table 11, several attempts to increase the PEI loading level were carried out. The percentage of nitrogen incorporated by the particles is below the 2.5-3.5% expected. In order to reach this value the focus was put on increasing the amount of phosphonate linked to the particles. First of all the phosphonate linking step has been carried out with Radley reactors instead of glass bottles on hotplate stirrer so that we had a better control of temperature. The phosphonate linking step was then performed by increasing the reaction temperature from 40° C. to 60° C. and 90° C.

In an additional experiment the pH of the carbonate buffer was increased from pH 9.8 to 10.96 as it was thought that that a more negatively charged linker could increase the loading of polymer, the quantity of PEI introduced was double that of the usual amount. Note that the decision to carry out these experiments on SNP008-2 was made because of their high rate of nitrogen incorporated shown in Table 12.

In the following table is displayed a summary of the conditions used:

TABLE 12
Experimental conditions

| Name | Heat | | Internal T | | pH | Details |
|---|---|---|---|---|---|---|
| 40C_PEI | 40 | C. | 37.5 | C. | 9.8 | Standard PEI |
| | | | | | | loading |
| 40C_PEI_× 2 | 40 | C. | 37.5 | C. | 9.8 | Doubled quantity |
| | | | | | | of PEI during |
| | | | | | | the PEI loading |
| 40C_PEI_pH + 1 | 40 | C. | 37.5 | C. | 9.8 | Carbonate buffer |
| | | | | | | pH + 1 (10.96 |
| | | | | | | instead of 9.8) |
| 60C_PEI | 60 | C. | 55 | C. | 9.3 | Standard PEI |
| | | | | | | Loading |
| 90C_PEI | 110 | C. | 90 | C. | ~9 | Standard PEI |
| | | | | | | loading |

C:H:N analysis results of this set of experiments are shown in table 13 below:

TABLE 13
Nitrogen and PEI content (wt %)

| Sample | N (wt %) | PEI content |
|---|---|---|
| 40C-PEI | 2.295 | 7.3% |
| 40C-PEI*2 | 1.973 | 6.3% |
| 40C-PH + 1 | 2.050 | 6.5% |
| 60C-PEI | 1.807 | 5.8% |
| 90C-PEI | 1.483 | 4.7% |

In general the nitrogen content increased compared to the results seen in table 11. It confirms that PEI loading is better with Radley reactors as heat is better distributed inside the solution. However as the table above shows, neither temperature increase, PET amount or carbonate pH increased the PEI content further. Actually when temperature increases the Nitrogen content is seen to decrease. This is explained by the fact that in the meantime pH decreases and it appears that reaction is more pH sensitive than temperature.

Conclusions

Work focused on process improvement using a Radley's reactor for precise control of process parameters. Significant progress has been made in reducing the process time and also the amount of materials used for the PEI loading. It was shown that the PEI amount can be reduced at least by 60% and the loading process need only last for 5 minutes. It was also determined that the time taken to perform the Phosphonate linking step might be reduced but further experiments would be required to confirm this finding. One other key observation relating to the temperature at which the process is carried out is that increasing the temperature decreases effectiveness of the process resulting in a lower polymer content. This parameter could be a useful control parameter if the PEI loading was needed to be set to a lower value if biocompatibility issues were of a concern.

### Example 3—Manufacture of SiNPs and Loading with Nucleic Acids

Methods

Synthesis and analysis of unloaded silica nanoparticles

Synthesis of unloaded silica nanoparticles—10 L scale

Reactions were carried out in 20 L Radley reactors equipped with an angled 4-bladed propeller and data logging capability for temperature, pH, conductivity, stirrer speed and torque.

The Radley Pilot reactor (20 L) was vacuumed down to approximately −0.75 bar and purged with nitrogen three times. Constant nitrogen gas was fed into the vessel at 0.1 mL/min. The vessel was charged with ethanol (8200 mL), water (1178 mL) and ammonium hydroxide (350 mL) and stirred (lid on) at 160 rpm. The reaction medium was then heated up to 45° C. Resorcinol (12.8702 g) was dissolved in ethanol (130 mL). Resorcinol and formaldehyde (18 mL) were added and the solution stirred (lid on) for 90 mins at 45° C. The temperature was lowered from 45° C. to 25° C. over a period of 35 mins. Tetraethyl orthosilicate (70 mL) was added and the mixture stirred (lid on) for 6 minutes.

Additional resorcinol (47.2829 g) was weighed out and dissolved in ethanol (100 mL). Resorcinol and formaldehyde (66 mL) were added and the solution stirred (lid on) for a further 2 hours.

The reaction mixture was transferred to a 15 L carboy. Four centrifuge bottles (Thermo Scientific Nalgene, 1 L) were filled with reaction mixture and centrifugation was carried out at 4700 rpm for 5 minutes at 10° C. Supernatant was removed, centrifuge bottles were filled up with more reaction mixture and centrifuged under the same conditions. Centrifuge steps were repeated until all reaction mixture had undergone the centrifugation process. Fresh ethanol (100 mL) was added to each bottle and centrifuged under the same conditions. Supernatant was removed and the crude sample was dried in air at ambient temperature for ˜17 hours.

Dried crude sample was transferred into a ceramic dish and placed into a furnace. The sample was heated from ambient temperature up to 550° C. at 2° C. per minute and the temperature held for 5 hours before cooling down naturally.

TABLE 14
Experimental amounts for synthesis of
silica nanoparticles - 10 L scale

| Reagent | SiNP NUMed (Batch 11(IV) |
|---|---|
| Ethanol/mL | 8230 |
| Water/mL | 1178 |
| Ammonium hydroxide/mL | 350 |
| Resorcinol/g [1^{st} addition] | 12.8702 |
| Formaldehyde/mL [1^{st} addition] | 18 |
| Tetraethyl orthosilicate/mL | 70 |
| Resorcinol/g [2^{nd} addition] | 47.2829 |
| Formaldehyde/mL [2^{nd} addition] | 66 |
| SiNP yield/g | 17.239 |

TABLE 15
Experimental process parameters for synthesis of silica nanoparticles - 10 L scale

| | | | | | Spike | | Spike |
|---|---|---|---|---|---|---|---|
| | Recipe | Scale of | Stirring | Polymerisation | growth | Polymerisation | growth |
| Experiment | used/nm | reaction/L | rate/rpm | temperature/° C. | temperature/° C. | time/min | time/min |
| SiNP | 180 | 10 | 160 | 45 | 25 | 132 | 120 |
| NUMed | | | | | | | |

SEW Analysis of Unloaded Silica Nanoparticles

SEM sample preparation: the sample was extracted from the vial and pressed onto a SEM stud with adhesive carbon tab, using the flat end of a spatula. SEM analysis: scanning electron microscopy was used to image all batches of SiNP using a Hitachi SU8230 instrument.

TEM analysis of unloaded silica nanoparticles

TEM analysis was completed using the following protocol: 10 μl of solution was dropped onto a carbon-coated 400 mesh copper grid. Excess solution was removed with a piece of filter paper and the grid was dried. The sample was viewed on a Philips CM100 TEM at 100 kV. Images were captured using a CCD camera Optronics 1824×1824 pixel with AMT40 version 5.42 image capture engine. The copper grids were supplied by Gilder grids and were carbon-coated using a Quorum Q150T ES coating unit.

BET Analysis of Unloaded Silica Nanoparticles

Analysis was carried out on Micromeritics TriStar II Plus and Micromeritics VacPrep 061 Sample Degas System using MicroActive for TriStar II Plus software.

The tubes were weighed when empty and sample was added to the tube using a metal funnel until the bulb was over half full. The tubes were weighed after filling. The tubes were put under vacuum at 80° C. for at least 12 hours. The tubes were weighed when degassed and set up for analysis.

Synthesis and Analysis of PEI Loaded Silica Nanoparticles

PEI Loading of Silica Nanoparticles—30 mg Scale

Silica nanoparticles (see mass in Table 16) were suspended in deionised water (10 mL) and sonicated for 10 minutes. 3-(Trihydroxysilyl propyl methyl phosphonate) (THPMP) (0.21 mL) was dissolved in deionised water (10 mL). The solutions were combined and stirred with a magnetic stirrer at 200 rpm, 40° C. for 2 hours. The resulting cloudy white solution was centrifuged at 10,000 rpm, 25° C. for 10 minutes. The supernatant was removed and the particles were suspended with deionised water (10 mL). The resulting solution was centrifuged at 10,000 rpm, 25° C. for 10 minutes. The supernatant was removed and the particles were suspended with carbonate buffer solution (5 mL, sodium carbonate (1.5926 g) and sodium bicarbonate (2.9333 g) in deionised water (1000 mL)).

Polyethylenimine (PEI) (see mass in Table 16) was dissolved in carbonate buffer solution (10 mL) by vigorous shaking. The solutions were combined and stirred with a magnetic stirrer at 200 rpm, 25° C. for 4 hours. The resulting cloudy white solution was centrifuged at 10,000 rpm, 25° C. for 10 minutes. The supernatant was removed and the particles were suspended with deionised water (10 mL). The resulting solution was centrifuged at 10,000 rpm, 25° C. for 10 minutes. The supernatant was removed and the particles were dried at room temperature, to give a solid white product.

TABLE 16
Experimental details for PEI loading
of silica nanoparticles - 30 mg scale

| | | | PEI SiNP |
|---|---|---|---|
| Sample | Mass/mg | PEI/mg | Product/mg |
| SiNP NUMed run 1 | 30.1 | 149.9 | 37.7 |
| SiNP NUMed run 2 | 30.0 | 149.7 | 31.5 |
| SiNP NUMed run 3 | 30.0 | 149.8 | 31.5 |

PEI Loading of Silica Nanoparticles—5 g Scale

Silica nanoparticles (see mass in Table 17) were suspended in deionised water (300 mL) and sonicated for 15 minutes. 3-(Trihydroxysilyl propyl methyl phosphonate) (THPMP) (12.8 mL) was dissolved in deionised water (300 mL). The solutions were combined and stirred with a magnetic stirrer at 500 rpm, 40° C. for 2 hours. The resulting cloudy white solution (with particles visible) was centrifuged at 10,000 rpm, 25° C. for 10 minutes. The supernatant was removed and the particles were suspended with deionised water (around 15 mL per tube). The resulting solution was centrifuged at 10,000 rpm, 25° C. for 10 minutes. The supernatant was removed and the particles were dried at room temperature, to give a solid white product.

A sample of the phosphonate loaded silica nanoparticles was taken and the remaining particles were suspended with carbonate buffer solution (200 mL). Polyethylenimine (PEI) (see mass in Table 17) was dissolved in carbonate buffer solution (300 mL) by vigorous shaking. The solutions were combined and stirred with a magnetic stirrer at 500 rpm, 25° C. for 4 hours. The resulting cloudy white solution with particles visible was centrifuged at 10,000 rpm, 25° C. for 10 minutes. The supernatant was removed and the particles were suspended with deionised water (around 15 mL per tube). The resulting solution was centrifuged at 10,000 rpm, 25° C. for 10 minutes. The supernatant was removed and the particles were dried at room temperature, to give a solid white product.

TABLE 17
Experimental details for PEI loading
of silica nanoparticles - 5 g scale

| | | | Phos SiNP | PEI SiNP |
|---|---|---|---|---|
| Sample | Mass/g | PEI/g | Product/g | Product/g |
| SiNP 0011 II run 1 | 5.9768 | 5.9932 | 0.2194 | 9.1942 |
| SiNP 0011 II run 2 | 6.0035 | 5.9991 | 0.1933 | 9.2820 |

DLS zeta potential analysis of PEI loaded silica nanoparticles

Analysis was carried out on a Horiba, Scientific Nanopartica, Nano Particle Analyzer, SZ 00 using Horiba SZ-100 software. Measurements were performed at 25° C., in water, in duplicate.

Silica nanoparticle samples (2 mg) were dispersed in deionised water (1 mL) to give white solid particles in a clear water solution. Samples were sonicated until there was a cloudy white solution with no solid white particles visible. 6 pipette drops of the sample were added to KCl solution (10⁻³ M, 100 mL). The electrode cell was filled with the resulting solution using a syringe (2 mL), ensuring no bubbles were visible in the cell and zeta potential was recorded.

Synthesis and Analysis of DNA/RNA and PEI Loaded Silica Nanoparticles

DNA Loading of PEI Loaded Silica Nanoparticles for DNA Quantification

A stock solution of PEI-SiNP particles (500 μg) in nuclease-free 10 mM phosphate buffered saline solution (1000 μL) was sonicated for 5 minutes, repipetted and then sonicated for a further 5 minutes to give a homogenous cloudy white solution. This solution was aliquoted (10 μL). DNA was made up into a stock solution to be able to aliquot 1 μL. DNA (1 μg) was added from the DNA stock solution and repipetted 3 times to mix. The solution was left static in a fridge at 5° C. for 4 hours. The solution was then microcentrifuged at 25° C. at 0,900 rpm for 13 minutes. The supernatant was pipetted out to be used for DNA quantification.

Positive control: DNA (1 μg) in nuclease-free 10 mM phosphate buffered saline solution (10 μL) was repipetted 3 times to mix. The solution was left static in a fridge at 5° C. for 4 hours. The solution was then microcentrifuged at 25° C. at 10,900 rpm for 13 minutes. The supernatant was pipetted out to be used for DNA quantification.

Negative control: Nuclease-free 10 mM phosphate buffered saline solution (10 μL).

UV-Vis Analysis of DNA and PEI Loaded Silica Nanoparticles for DNA Quantification

The DNA concentration in the supernatant was determined using a NanoDrop 8000 spectrophotometer and 2 μl of sample.

DLS Zeta Potential Analysis of DNA and PEI Loaded Silica Nanoparticles

Analysis was carried out on a Horiba, Scientific Nanopartica, Nano Particle Analyzer, SZ-100 using Horiba SZ-100 software. Measurements were performed at 25° C., in 10 mM phosphate buffered saline solution, in duplicate.

A solution of PEI-SiNP particles (100 μg) in nuclease-free 10 mM phosphate buffered saline solution (200 μL) was sonicated for 5 minutes, repipetted and then sonicated for a further 5 minutes to give a homogenous cloudy white solution. DNA was made up into a stock solution to be able to aliquot 1 μL. DNA (10 μg) was added from the DNA stock solution and repipetted 3 times to mix. The solution was left static in a fridge at 5° C. for 4 hours. The solution was then microcentrifuged at 25° C. at 10,900 rpm for 13 minutes. The resulting product was re-suspended in 10 mM phosphate buffered saline solution (5 mL). The electrode cell was filled with the resulting solution using a syringe (2 mL), ensuring no bubbles were visible in the cell and zeta potential was recorded.

It should be noted here that for all DNA and RNA loading zeta potential analysis, not all equipment can be ensured to be nuclease-free, however measures were taken to attempt to make the experiment as nuclease-free as possible.

RNA Loading of PEI Loaded Silica Nanoparticles for RNA Quantification

A stock solution of PEI-SiNP particles (500 μg) in nuclease-free 10 mM phosphate buffered saline solution (1000 μL) was sonicated for 5 minutes, repipetted and then sonicated for a further 5 minutes to give a homogenous cloudy white solution. This solution was aliquoted (10 μL) and RNA (1 μg) was added directly from the raw material and repipetted 3 times to mix. The solution was left static in a fridge at 5° C. for 4 hours. The solution was then microcentrifuged at 25° C. at 10,900 rpm for 3 minutes. The supernatant was pipetted out to be used for RNA quantification.

Positive control: RNA (1 μg) in nuclease-free 10 mM phosphate buffered saline solution (10 μL) was repipetted 3 times to mix. The solution was left static in a fridge at 5° C. for 4 hours. The solution was then microcentrifuged at 25° C. at 10,900 rpm for 3 minutes. The supernatant was pipetted out to be used for RNA quantification.

Negative control: Nuclease-free 10 mM phosphate buffered saline solution (10 μL).

Fluorescence Analysis of RNA and PEI Loaded Silica Nanoparticles for RNA Quantification

The RNA concentration in the supernatant was determined using a Qubit 3.0 fluorometer and the Qubit RNA BR kit. The samples and kit RNA standards were mixed with the Qubit dye and analysed using the RNA Broad Range Assay program on the Qubit 3.0 fluorometer.

DLS Zeta Potential Analysis of RNA and PEI Loaded Silica Nanopartieles

Analysis was carried out on a Horiba, Scientific Nanopartica, Nano Particle Analyzer, SZ-100 using Horiba SZ-100 software. Measurements were performed at 25° C., in 10 mM phosphate buffered saline solution, in duplicate.

A solution of PEI-SiNP particles (100 μg) in nuclease-free 10 mM phosphate buffered saline solution (200 μL) was sonicated for 5 minutes, repipetted and then sonicated for a further 5 minutes to give a homogenous cloudy white solution. RNA (10 μg) was added directly from the raw material and repipetted 3 times to mix. The solution was left static in a fridge at 5° C. for 4 hours. The resulting product was re-suspended in 10 mM phosphate buffered saline solution (4.8 mL). The electrode cell was filled with the resulting solution using a syringe (2 mL), ensuring no bubbles were visible in the cell and zeta potential was recorded.

In-Vitro DNA Loading of PEI Loaded Silica Nanoparticles for Stability Testing

A stock solution of PEI-SiNP particles (500 rig) in nuclease-free 10 mM phosphate buffered saline solution (1250 μL) was sonicated for 5 minutes, repipetted and then sonicated for a further 5 minutes to give a homogenous cloudy white solution. This solution was aliquoted (100 μL). DNA was made up into a stock solution to be able to aliquot 1 μL. DNA (4 μg) was added from the DNA stock solution and repipetted 3 times to mix. The solution was agitated on a plate shaker at 550 rpm for 6 hours at 4° C. in an ice bath. The mixture was sampled at 0, 2 and 6 hour time points for DNA quantification. At 6 hours, the solution was then microcentrifuged at 25° C. at 10,900 rpm for 13 minutes. The supernatant was sampled for DNA quantification and snap-frozen at 6 hour time point for capillary electrophoresis.

Positive control: DNA (4 μg) in nuclease-free 10 mM phosphate buffered saline solution (100 μL) was repipetted 3 times to mix. This was carried out in duplicate and one sample was sampled for DNA quantification and snap-frozen immediately for capillary electrophoresis. The other solution was agitated on a plate shaker at 550 rpm for 6 hours at 4° C. in an ice bath. The mixture was sampled at 0, 2 and 6 hour time points for DNA quantification. At 6 hours, the solution was then microcentrifuged at 25° C. at 10,900 rpm for 13 minutes. The supernatant was sampled for DNA quantification and snap-frozen at 6 hour time point for capillary electrophoresis.

Negative control: Nuclease-free 10 mM phosphate buffered saline solution (100 μL).

In-Vitro RNA Loading of PEI Loaded Silica Nanoparticles for Stability Testing

A stock solution of PEI-SiNP particles (500 μg) in nuclease-free 10 mM phosphate buffered saline solution (1250 μL) was sonicated for 5 minutes, repipetted and then sonicated for a further 5 minutes to give a homogenous cloudy white solution. This solution was aliquoted (100 μL) and RNA (4 μg) was added directly from the raw material and repipetted 3 times to mix. The solution was agitated on a plate shaker at 550 rpm for 6 hours at 4° C. in an ice bath. The mixture was sampled at 0, 2 and 6 hour time points for DNA quantification and snap-frozen at 6 hour time point for capillary electrophoresis.

Positive control: RNA (4 μg) in nuclease-free 10 mM phosphate buffered saline solution (100 μL) was repipetted 3 times to mix. This was carried out in duplicate and one sample was sampled for RNA quantification and snap-frozen immediately for capillary electrophoresis. The other solution was agitated on a plate shaker at 550 rpm for 6 hours at 4° C. in an ice bath. The mixture was sampled at 0, 2 and 6 hour time points for RNA quantification and snap-frozen at 6 hour time point for capillary electrophoresis.

Negative control: Nuclease-free 10 mM phosphate buffered saline solution (100 μL).

In-Vivo DNA Loading of PEI Loaded Silica Nanoparticles for Stability Testing

A solution of PEI-SiNP particles (500 μg) in 0.22 μm filtered 0.5% (w/v) hydroxymethylcellulose in nuclease-free 10 mM phosphate buffered saline solution (100 μL) was agitated on a plate shaker for 1 minute at 500 rpm. The solution was then sonicated for 10 minutes, repipetted, sonicated for a further 10 minutes, repipetted and then sonicated for a further 10 minutes to give a homogenous cloudy white solution. DNA was made up into a stock solution to be able to aliquot 1 μL. DNA (50 μg) was added from the DNA stock solution and repipetted 3 times to mix. The solution was agitated on a plate shaker at 550 rpm for 6 hours at 4° C. in an ice bath. The mixture was sampled at 0, 2 and 6 hour time points for DNA. At 6 hours, the solution was then microcentrifuged at 25° C. at 10,900 rpm for 13 minutes. The supernatant was sampled for DNA quantification and snap-frozen at 6 hour time point for capillary electrophoresis.

Positive control: DNA (50 μg) in 0.22 μm filtered 0.5% (w/v) hydroxymethylcellulose in nuclease-free 10 mM phosphate buffered saline solution (100 μL) was repipetted 3 times to mix. This was earned out in duplicate and one sample was sampled for DNA quantification and snap-frozen immediately for capillary electrophoresis. The other solution was agitated on a plate shaker at 550 rpm for 6 hours at 4° C. in an ice bath. The mixture was sampled at 0, 2 and 6 hour time points for DNA. At 6 hours, the solution was then microcentrifuged at 25° C. at 10,900 rpm for 13 minutes. The supernatant was sampled for DNA quantification and snap-frozen at 6 hour time point for capillary electrophoresis.

Negative control: 0.22 μm filtered 0.5% (w/v) hydroxymethylcellulose nuclease-free 10 mM phosphate buffered saline solution (100 μL).

In-Vivo RNA Loading of PET Loaded Silica Nanoparticles for Stability Testing

A solution of PEI-SiNP particles (500 μg) in 0.22 μm filtered 0.5% (w/v) hydroxymethylcellulose in nuclease-free 10 mM phosphate buffered saline solution (100 μL) was agitated on a plate shaker for 1 minute at 500 rpm. The solution was then sonicated for 10 minutes, repipetted, sonicated for a further 10 minutes, repipetted and then sonicated for a further 10 minutes to give a homogenous cloudy white solution. RNA (50 μg) was added directly from the raw material and repipetted 3 times to mix. The solution was agitated on a plate shaker at 550 rpm for 6 hours at 4° C. in an ice bath. The mixture was sampled at 0, 2 and 6 hour time points for RNA quantification and snap-frozen at 6 hour time point for capillary electrophoresis.

Positive control: RNA (50 μg) in 0.22 μm filtered 0.5% (w/v) hydroxymethylcellulose nuclease-free 10 mM phosphate buffered saline solution (100 μL) was repipetted 3 times to mix. This was carried out in duplicate and one sample was sampled for DNA quantification and snap-frozen immediately for capillary electrophoresis. The other solution was agitated on a plate shaker at 550 rpm for 6 hours at 4° C. in an ice bath. The mixture was sampled at 0, 2 and 6 hour time points for RNA quantification and snap-frozen at 6 hour time point for capillary electrophoresis.

Negative control: 0.22 μm filtered 0.5% (w/v) hydroxymethylcellulose nuclease-free 10 mM phosphate buffered saline solution (100 μL).

Capillary Electrophoresis (CE) for DNA Analysis

Prior to CE analysis the supernatant plasmid DNA Samples (ovalbumin (OVA) pDNA and human papilloma virus (HPV) pDNA) were digested with BamHI enzyme and purified using Monarch PCR & DNA clean up Kit. The pre-treated plasmid DNA samples were then run on a Lab Chip GXii system. The samples were analysed using the DNA 5K reagent kit from PerkinElmer. A lower and higher molecular weight marker (present in the kit marker buffer) were run with each of the samples. A molecular weight marker (DNA ladder from the DNA 5K reagent kit) was run alongside the samples.

Capillary Electrophoresis for RNA Analysis

The supernatant mRNA samples (OVA mRNA) were run on a Lab Chip GXii system. The samples were analysed using the RNA pico assay reagent kit from PerkinElmer. The mRNA samples were pre-treated with the RNA pico assay reagent kit sample buffer and heated at 70° C. for 2 minutes. A lower molecular weight marker (present in the kit sample buffer) was run with each of the samples. A molecular weight marker (RNA ladder from the RNA Pico Assay Reagent Kit) was run alongside the samples.

Results and Discussion

Synthesis, Characterisation and Loading of Nanoparticles

Synthesis of 10 L Batch of Silica Nanoparticles

A 10 L batch of silica nanoparticles was prepared and is referred to as SiNP NUMed (Batch 11(IV)). The resulting blank silica nanoparticles were characterised by SEM for particle size and appearance.

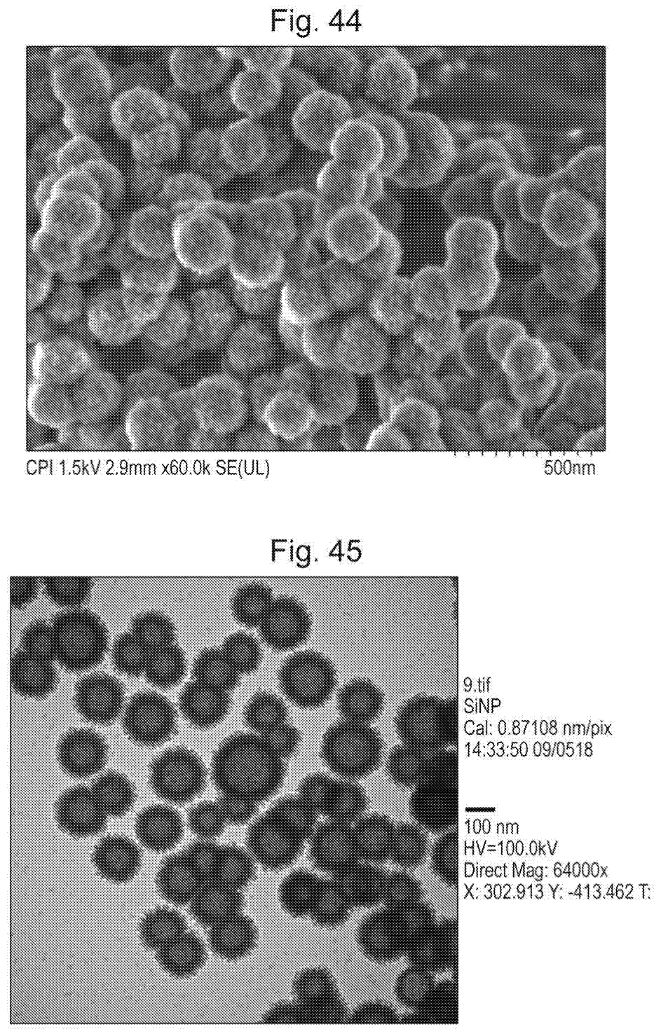

FIG. 44 shows an SEM image for SiNP NUMed. Particles analysis was carried out on SEM images and the results calculated show the particles have an average particle size of 203±25 nm (count 161, standard deviation 24.6 nm, mode 204 nm). Uniform particles (PDI 0.12) were observed in the SEM images and the particles appear to have the desired spiky surface morphology.

FIG. 45 shows a TEM image for SiNP NUMed. An average particle diameter of 195 nm was calculated from analysis of TEM images. The average core diameter was calculated as 96 nm and the average shell thickness was calculated as 51.35 nm.

The surface area of the particles is important for PEI and subsequent nucleic acid loading and is determined by Brunauer-Emmett-Teller (BET) nitrogen sorption. A surface area of 172 m²/g was determined for the SiNP NUMed nanoparticles.

PEI Loading of Silica Nanoparticles at 30 mg Scale

The zeta potentials of PEI SiNP NUMed runs 1, 2 and 3 were found to be 3.9, 7.9 and 22.2 mV respectively. This indicates loading of PEI.

PEI Loading of Silica Nanoparticles at 5 g Scale

The zeta potentials of PEI SiNP 0011 II runs 1 and 2 were found to be 18.1 and 15.8 mV respectively. This indicates loading of PEI.

DNA Loading of PEI Loaded Silica Nanoparticles

Batches of PEI loaded SiNP nanoparticles were loaded with eGFP pDNA in triplicate. Table 18 shows that the particles were successfully loaded with eGFP DNA.

TABLE 18
Results for DNA quantification of eGFP DNA loading

DNA loading on particles ng/μg

| | Relative to positive | Relative to positive |
|---|---|---|
| | control measured | control measured |
| Sample | at 0 hours | at 4 hours |
| Experiment 1 | 135 | 136 |
| Experiment 2 | 144 | 145 |
| Experiment 3 | 130 | 131 |

Loading and Quantification of OVA and HPV Nucleic Acids (pDNA and mRNA)

Loading of OVA pDNA, HPV pDNA and OVA mRNA was tested by analysing DNA/RNA concentration in solution to back calculate the concentration on the particle surface vs a positive control. Zeta potential analysis was also used to conth in a change in the surface charge on the particles from positive (PEI) to negative (nucleic acid).

OVA pDNA Loading of PEI Loaded Silica Nanoparticles

PEI loaded SiNPs were loaded with OVA DNA in triplicate. Loading in the target range of 100-140 ng/μg vs positive controls at 0 and 4 hour time points was achieved as shown in Table 19.

TABLE 19
Results for DNA quantification of OVA DNA loading

DNA loading on particles/ng/μg

| | Relative to positive | Relative to positive |
|---|---|---|
| | control measured | control measured |
| Sample | at 0 hours | at 4 hours |
| Experiment 1 | 138 | 136 |
| Experiment 2 | 150 | 148 |
| Experiment 3 | 142 | 140 |

Zeta potential analysis of particles loaded with OVA DNA shows the expected negative surface charge (−8.8 mV) indicative of nucleic acid loading.

HPV pDNA Loading of PEI Loaded Silica Nanoparticles

Successful loading of PEI loaded SiNP nanoparticles with HPV pDNA was observed. The results of the pDNA loading are shown in Table 20.

TABLE 20
Results for DNA quantification of HPV DNA loading

DNA loading on particles/ng/μg

| | Relative to positive | Relative to positive |
|---|---|---|
| | control measured | control measured |
| Sample | at 0 hours | at 4 hours |
| Experiment 1 | 214 | 217 |
| Experiment 2 | 214 | 217 |
| Experiment 3 | 224 | 227 |

The corresponding zeta potential analysis of particles loaded with HPV DNA shows the expected negative surface charge (−7.8 mV) indicative of nucleic acid loading.

OVA mRNA Loading of PEI Loaded Silica Nanoparticles

Loading experiments were repeated using OVA mRNA, with analysis carried out using the Qubit fluorescence assay. Due to the concerns over stability of mRNA during centrifugation at ambient temperature centrifugation was carried out for a shorter period of time (3 mins) than used for DNA (13 mins).

OVA mRNA was found to successfully load onto the PEI-loaded SiNPs. The loading results are shown in Table 21.

TABLE 21
Results for RNA quantification of
OVA RNA loading on NV00100018

RNA loading on particles/ng/μg

| | Relative to positive | Relative to positive |
|---|---|---|
| | control measured | control measured |
| Sample | at 0 hours | at 4 hours |
| Experiment 1 | 244 | 169 |
| Experiment 2 | 243 | 168 |
| Experiment 3 | 239 | 164 |

The zeta potential analysis for OVA mRNA is similar to the results from OVA and HPV pDNA, showing a negative surface charge (−6.7 mV) indicative that the particle surface has been modified by mRNA.

Stability of pDNA and mRNA Loaded on PEI-SiNPs

The stability of the pDNA and mRNA loaded onto the PEI-SiNPS was assessed six hours after loading by DNA quantification and capillary electrophoresis. It was found that the OVA pDNA, OVA mRNA and HPV pDNA all remained successfully loaded on the SiNP after 6 hours and that there was no degradation observed for the pDNA or mRNA.

### Example 4—Effect of OVA pDNA Loaded SiNP on Splenocyte Proliferation

The effect of SiNP hollow nanoparticles loaded with different amounts of OVA pDNA (Ram-DNA) in causing an immune response was assessed in a mouse splenocyte proliferation and compared with control (PBS), OVA (ovalbumin protein), OVA-CFA (ovalbumin protein/complete Freund's adjuvant), pDNA (ovalbumin DNA alone), JET-DNA (ovalbumin DNA/JET PEI transfection agent) and unloaded SiNPs (Ram-75 mg/kg). The mice were immunised at 0, 7 and 14 days and spleens were collected on day 28 for splenocyte isolation. The Splenocytes were seeded in 96-well plates with and without OVA stimulation for 48 hours. MTT analysis was conducted to assay relative numbers of splenocytes in triplicate with six mice per group.

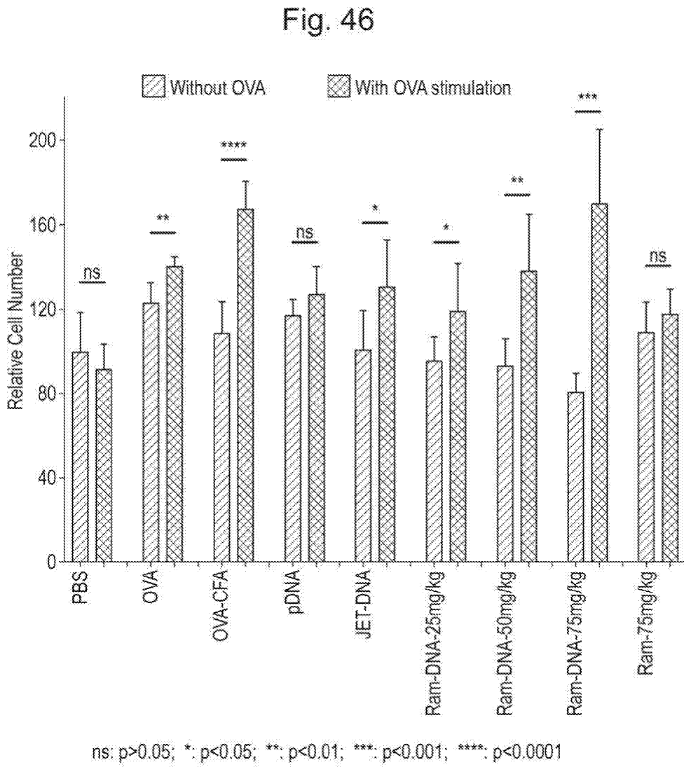

The results are shown in FIG. 46. It can be seen that the SiNP hollow nanoparticles lead to increased stimulation of splenocyte proliferation.

### Example 5—Transfection of Cancer Cell Lines Using SiNPs

Hollow SiNPs were loaded with pGL4.13[luc2/SV40] plasmid DNA (obtained from Promega). pGL4.13[luc2/SV40] pDNA encodes luciferase and can be used to detect successful transfection by luminescence.

Transfection efficiency 48 hours after transfection was assessed in three cell lines: CT26, HCTI 16 and HEK293. CT26 is a mouse colon carcinoma cell line often used as a cancer model. CT26 cells share molecular features with aggressive, undifferentiated, refractory human colorectal carcinoma cells. HCT116 is a human colon cancer cell line used in therapeutic research and drug screenings. HEK293 is a permanent cell line established from primary embryonic human kidney cells. It is used to produce recombinant DNA or gene products and for production of viruses for cell therapy.

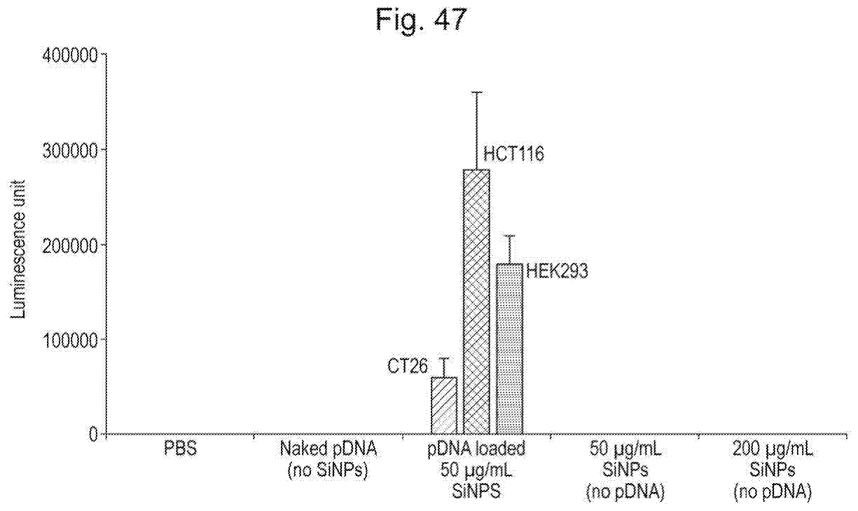

The results of the transfection in the three cell lines are shown in FIG. 47. Transfection with pDNA loaded SiNPs was compared with naked pDNA and SiNPs without pDNA. It was found that the SiNPs allowed successful transfection of the luciferase gene into three different cell types, two of which are models for cancer treatment and one of which is commonly used in production of cell therapy vectors.

### Example 6—Surface Modification of SiNPs

Synthesis of Ram-SNPs with Diameter of Approx. 330 nm

Resorcinol (0.2 g) and formaldehyde (37 wt %, 0.28 mL) were added to the solution composed of ammonia aqueous solution (28 wt %, 3.0 mL), deionized water (10 mL) and ethanol (70 mL). The mixture was vigorously stirred for 6 h at room temperature, then 0.6 mL of tetraethylorthosilicate (TEOS) was added to the solution and stirred for 8 minutes before the second addition of resorcinol (0.4 g) and formaldehyde (37 wt %, 0.56 mL). The mixture was stirred for 2 h at room temperature, and then transferred into an autoclave for hydrothermal treatment at 150° C. for 24 h. The RF-silica particles were then collected by centrifugation, washed with ethanol and dried at 50° C. Finally, Ram-SNPs were collected after calcination at 550° C. for 5 h in air (where “Ram” refers to a rambutan-like structure).

Synthesis of Smooth Silica Nanoparticles (S-SNPs), Raspberry Silica Nanoparticles (Ras-SNPs) and Flower-Like Silica Nanoparticles (Flw-SNPs).

For the synthesis of S-SNPs, resorcinol (0.2 g) and formaldehyde (37 wt %, 0.28 mL) were added to the solution composed of ammonia aqueous solution (28 wt %, 3.0 mL), deionized water (10 mL) and ethanol (70 mL). The mixture was vigorously stirred for 6 h at room temperature, then 1.4 mL of TEOS was added into the solution and stirred for 2 h before centrifugation to collect the solid product. For the synthesis of Ras-SNPs, resorcinol (0.2 g) and formaldehyde (37 wt %, 0.28 mL) were added to the solution composed of ammonia aqueous solution (28 wt %, 3.0 mL), deionized water (10 mL) and ethanol (70 mL). The mixture was vigorously stirred for 18 h at room temperature, then 0.6 mL of TEOS was added into the solution and stirred for 2 h before centrifugation to collect the solid product. For the synthesis of Flw-SNPs, the protocol is based on our previous publication. 9 mL of Milli-Q water, 0.3 g of TEA and 1 mL of CTAC solution were mixed at 60° C. for 1 h, followed by the addition to the mixture of 9.5 mL of chlorobenzene and 25 μL of APTES. The reaction solution was kept under stirring at 60° C. for 1 h. Then, 0.5 mL of TEOS was added in the reaction solution and stirred for 24 h. The solid sample was collected by centrifugation at 20,000 rpm for 10 min and then washed with ethanol. All samples were further calcined at 550° C. for 5 h in air to remove the template or surfactant.

Characterisation

The morphology of silica nanoparticles was characterised by transmission electron microscopy (TEM) using a JEOL 1010 microscope operated at 100 kV. Nitrogen sorption analysis was conducted using a Microrneritics Tristar 3020. Before measurement, all samples were degassed under vacuum 80° C. for at least 12 h. The pore size distribution was calculated according to the Barret-Joyner-Halenda (BJH) method derived from the adsorption branch. The zeta potential of the silica nanoparticles was measured in PBS using a Zetasizer Nano-ZS from Malvern Instrument. The nitrogen content in PEI-conjugated nanoparticles was determined by CHNS-O Elemental Analyzer using a Thermo Flash EA1112 Series.

Results

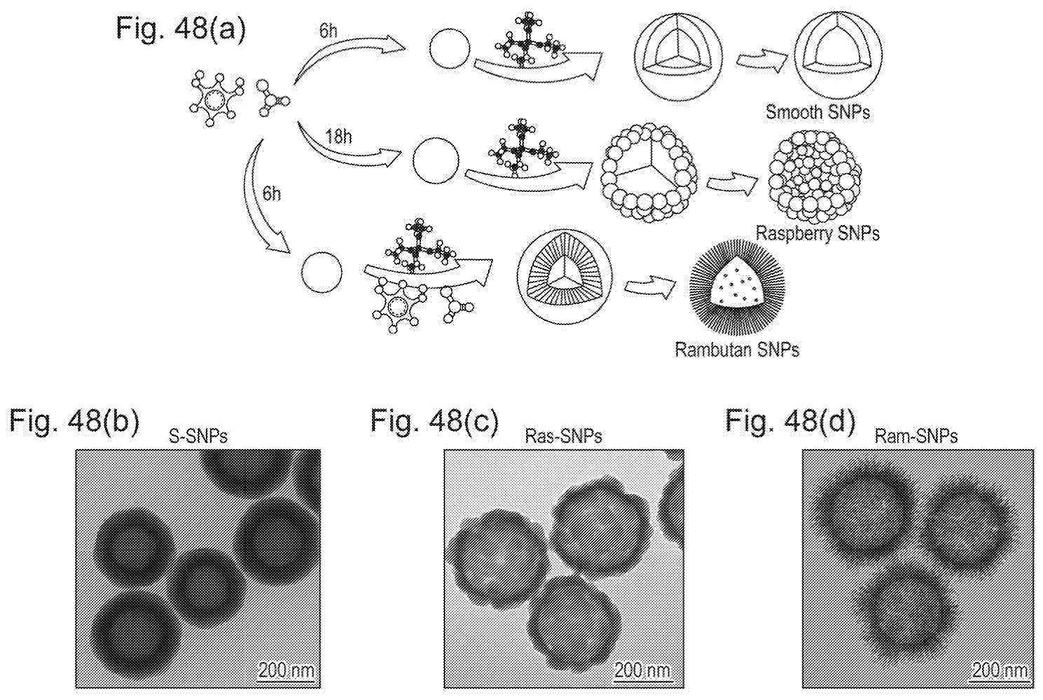

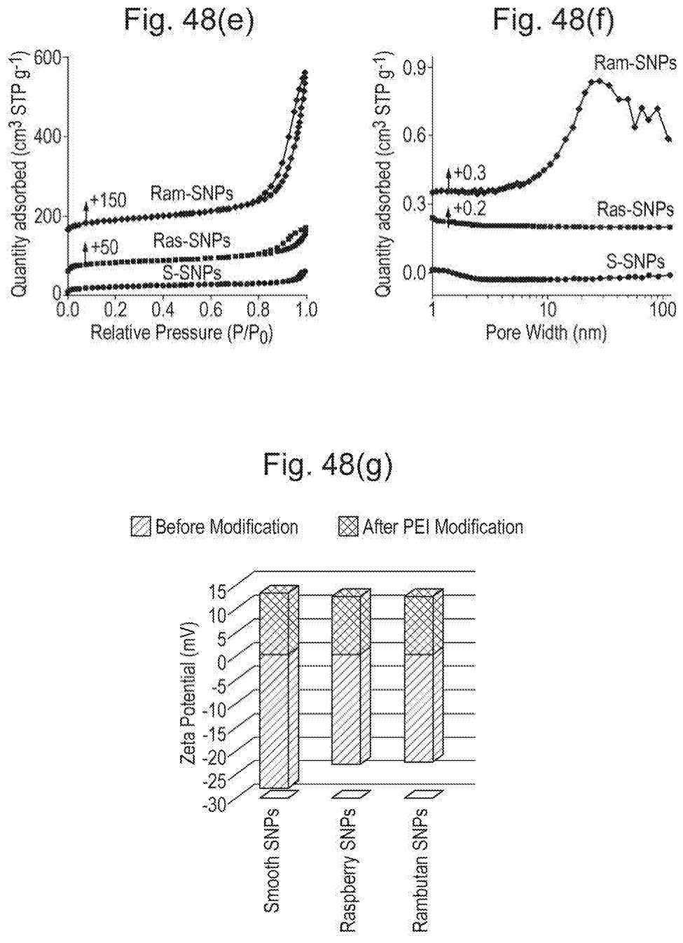

S-SNPs, Ras-SNPs and Ram-SNPs can be obtained using the RF-silica synthesis system by varying the synthesis parameters and the TEM images (FIG. 48 b-d) clearly show their surface topology. The particle size of these three types of SNPs were similar, ranging from 310 to 350 nm as calculated from TEM. The nitrogen sorption analysis results are shown in FIGS. 48 e-f, where Ram-SNPs exhibited a surface area of 142 m²/g, pore volume of 0.64 cm³/g and pore size of larger than 20 nm. The surface charge of bare silica nanoparticles was negative, as shown in FIG. 48 g. The zeta potential of these silica nanoparticles changed from around −20˜−30 mV to +10 mV after PEI conjugation.

Selection of Surface Functionalisation Approach

It is well understood that transfection is more effective when particles crossing a cell membrane barrier are positively charged. This is due to the favourable interaction positively charged particles have with the negatively charged surfaces of cell membranes. Surface modification approaches therefore focussed on different approaches for rendering a positive charge on the inherently negatively charged surface of naked silica. Varying types of polyethylene imine (PEI) modification were explored as this agent is well known for its ability to create positively charged surfaces.

Methodology

PEI Modification of Silica Nanoparticles—Covalent Binding of PEI Via Epoxy Groups

In this group, varying sizes of PEI molecules were attached to the surface of the silica particles by covalent bonding of the PEI molecules with epoxy groups attached to the surface of the silica. 100 mg of silica nanoparticles were immersed into 30 mL of toluene and then refluxed at 70° C. for 15 min under stirring and nitrogen gas blanket protection. Then 1.5 mL of (3-glycidyloxypropyl) trimethoxysilane (3-GPS) was added into the solution to generate a silica surface populated with epoxy groups and further refluxed for 24 h. The solid products were collected by centrifugation at 10,000 rpm for 10 min and washed twice, first using toluene and then with methanol. The particles with epoxy groups were then dried in air at room temperature. 50 mg of epoxy group-modified silica nanoparticles were mixed with 250 mg of PEI molecules (different molecular weights: 1.8 k, 10 k and 25 k) in 100 mL of 50 mM (pH 9.5) carbonate buffer solution. The mixture was stirred for 24 h, then solid products were collected by centrifugation and water washing. The solid products were then resuspended into 20 mL of 1 g/L (pH 9) ethanolamine solution and stirred for 6 h at room temperature. The final PEI modified particles were harvested by centrifugation, purified by water/ethanol washing and dried at room temperature.

PEI Modification of Silica Nanoparticles—Strong Electrostatic Attraction Via Phosphonate Groups

An alternative method of PEI attachment to the silica using strong electrostatic attraction with the PEI was explored. This used phosphonate groups bound to the silica surface to electrostatically bond with the PEI molecules. To attach the phosphonate surface groups, 30 mg of silica nanoparticles were dispersed into 10 mL of water and the pH was adjusted to 10 using ammonium hydroxide. Then 10 mL of the particle solution mixed with 10 mL of 56 mM of 3-(Trihydroxysilyl) propylmethylphosphonate (THPMP) solution for surface phosphonate modification by stirring at 40° C. for 2 h. The solid products were collected by centrifugation, and thoroughly washed with water. The solid product was then resuspended in 15 mL of water or ethanol containing 150 mg of PEI molecules. After stirring for 4 h at room temperature, the PEI modified nanoparticles were obtained by centrifugation, water washing and room temperature drying.

Results

Bare silica nanoparticles had the expected negative surface charge, which is not ideal for the adsorption of negatively charged pcDNA. To achieve strong binding between silica nanoparticles and pcDNA, PEI was conjugated to the silica nanoparticle surfaces to render a positive surface charge. However, there are various approaches to conjugate PEI on silica nanoparticles including covalent binding and strong electrostatic attraction. Silica nanoparticles were modified according to these two

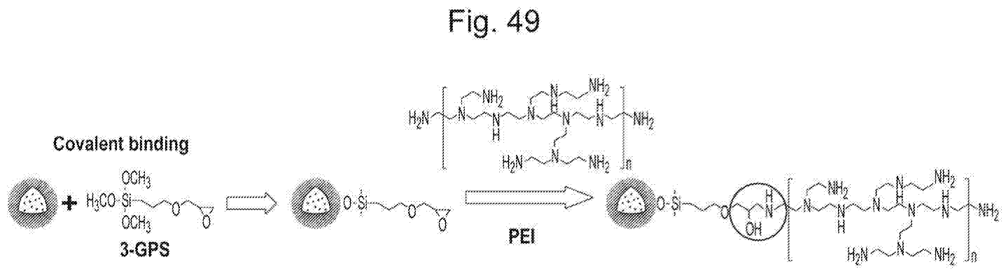

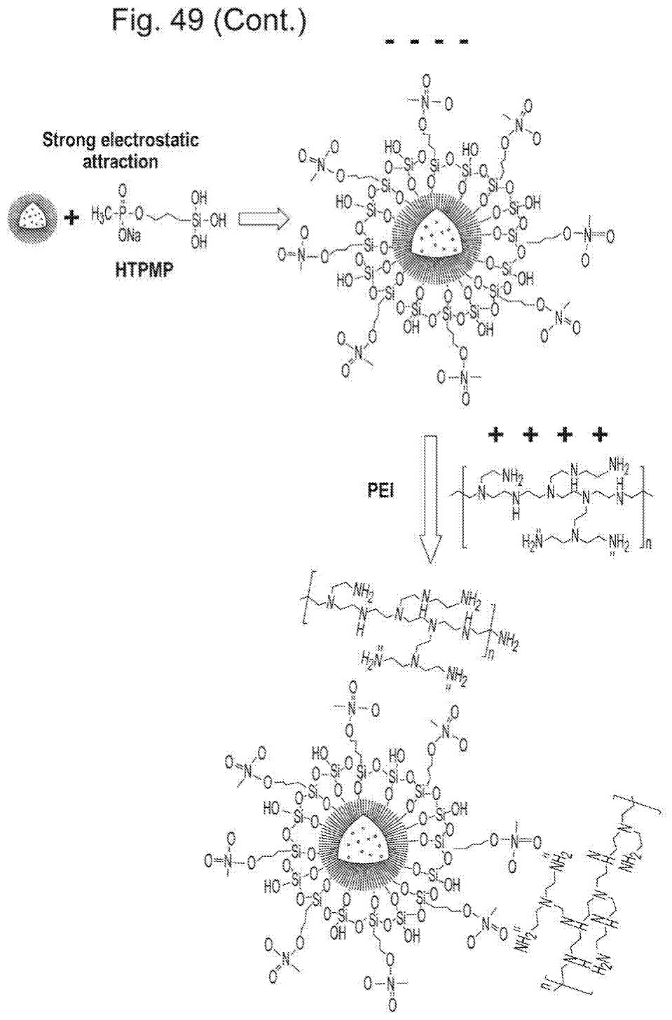

PEI conjugation modes for comparison. As shown in FIG. 49, silica nanoparticles were first modified by 3-GPS, attaching epoxy groups to the particle surface which can further form covalent bonds with the amino groups on the PEI molecule. Alternatively, the surfaces of the silica nanoparticles were modified with THPMP, attaching numerous phosphonate groups to the silica surface, further enhancing the negative surface charge of silica nanoparticles and enabling a strong electrostatic attraction with the positively charged PEI molecules.

The amount of PEI attached during modification was analysed by elemental analysis of the particles after conjugation. As there are no nitrogen atoms contained in the bare silica nanoparticles or in 3-GPS/THPMP modified particles, the only nitrogen content is contributed from PEI attached to the particles. As shown in Table 22, the nitrogen content across the four types of particles tested showed the tendency of Ram-SNPs>Ras-SNPs>S-SNPs, which may be attributed to their surface area differences. Comparing the two types of PEI conjugation, nanoparticles after phosphonate modification bind more PEI on the surface. Besides these two types of PEI conjugation, the physical adsorption of PEI on silica nanoparticles surface was also tested, which showed 3.1% nitrogen content in the particles. However physically adsorbed PEI is expected to be less strongly bound that PEI attached by 3-GPS and THPMP.

TABLE 22

Nitrogen content (%)

| | Epoxy-PEI | Phosphonate-PEI |
|---|---|---|
| S-SNPs | 0.5 | 0.8 |
| Ras-SNPs | 1.1 | 2.4 |
| Ram-SNPs | 2.6 | 3.2 |

Apart from the PEI conjugation mode, the molecular weight of PEI also affects the pcDNA binding and transfection efficiency. Here, PEI with molecular weights of 1.8 k, 10 k and 25 k were covalently conjugated with silica nanoparticles for further comparison.

pcDNA Loading and Gel Electrophoresis

Methodology

pcDNA Loading

1 μg of pcDNA was mixed with 5 μg of PEI covalently modified silica nanoparticles in 10 μL of PBS solution at 4° C. for 4 h. Afterwards, the mixture was centrifuged at 15,000 rpm for 10 min and the supernatant was used for pcDNA residual amount quantification via Nanodrop.

Gel Electrophoresis

0.5 μg of pcDNA was mixed with silica nanoparticles at silica dosages ranging from 0 to 5, 10, 20, 40 and 60 μg. The mixtures were incubated at 4° C. for 4 h and then 2 mL of nucleic acid sample buffer was added into the mixture forming a total solution volume of 10 μL. To prepare agarose gel, 2.5 g of ultrapure agarose was added into 250 mL of Milli-Q water, then boiled under microwave irradiation to fully dissolve the agarose. After the agarose solution had cooled down, 25 μL of SYBR-Safe gel stain (10,000×) was added into the solution. The solution was finally poured into the gel container and cooled for 20 min to form the gel. The gel container with gel was transferred into the tank and filled with TEA buffer to immerse the gel. Then 10 μL of the pcDNA solution was injected into the pores of the gel one by one, and the voltage was set to 80 V for electrophoresis for 50 min. The gel after electrophoresis was recorded one by one.

Results

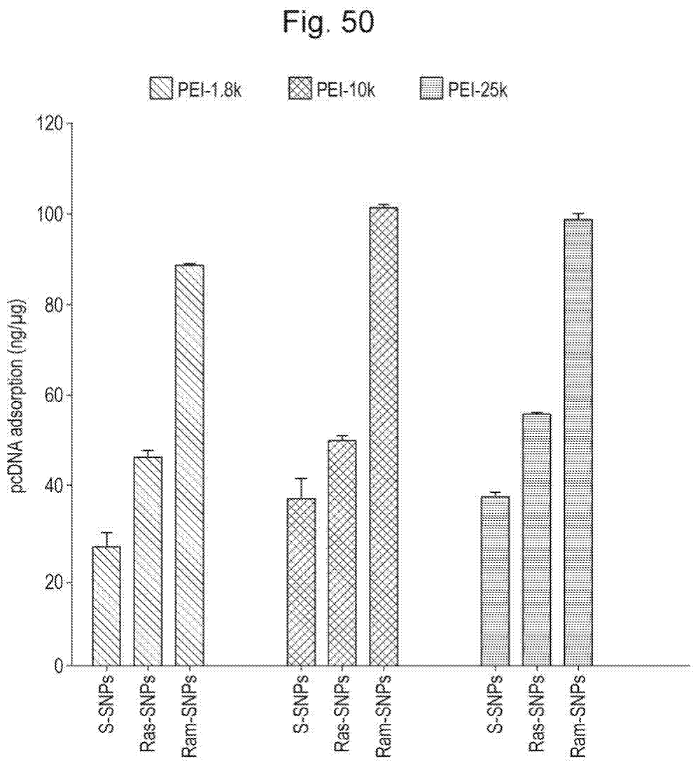

GFP expressing pcDNA with a molecular weight of 6.1 kD was employed in this study. The loading capacity of pcDNA on the silica nanoparticles, which were covalently bind with PEI, was investigated. As shown in FIG. 50, the Ram-SNPs modified with different molecular weights of PEI showed the highest DNA loading capacity of around 100 ng/μg. However, S-SNPs and Ras-SNPs could only achieve loadings of less than 50 ng/μg. This may result from the difference in their surface area and pore volume to accommodate pcDNA. The rambutan-like structure of Ram-SNPs may favour rope-like pcDNA entanglement in the surface spikes, enabling easy and firm binding with pcDNA in solution.

To further demonstrate the difference of binding affinity between PEI-modified silica nanoparticles and pcDNA, gel electrophoresis was used for comparison. Across all groups, increasing the ratio of silica nanoparticles to pcDNA (decreasing the pcDNA loading), decreased the amount of pcDNA released. This makes sense from an intuitive point of view in that the pcDNA at low loading levels is closely bound to the particle surface and therefore will be tightly bound, As more pcDNA is loaded onto particles, it is expected that the additional layers of pcDNA loaded will be less strongly bound as these outer layers are increasingly associated with the underlying pcDNA layers and not the modified silica surface which is designed to adhere pcDNA strongly. This finding may have implications for transfection efficiency in that efficacious transfection will likely be hindered if pcDNA is too tightly bound to the silica particles and unable to be released once the particles enter cells. Transfection efficiency may be promoted therefore by increasing the pcDNA loading on the silica particles.

The larger the molecular weight of PEI conjugated on the surface of silica nanoparticles, the stronger the binding affinity can be achieved for most of the particles measured. Comparing the four types of silica particles, pcDNA band release from the formulations can always be identified at all loading levels. Ras-SNPs showed only slightly improved binding affinity relative to S-SNPs, which indicated the weak binding between pcDNA and S-SNP surface. Ram-SNPs modified with 10 k PEI showed the strongest binding with no pcDNA release at a pcDNA/SiO₂ weight ratio of 1/10.

Screening Particle Library for Transfection Efficiency in HEK-293 Cells

The well-known HEK-293 cell line was used to compare the in vitro transfection efficiency of the above silica/pcDNA variants and Lipofectamine 2000 commercial reagent.

Methodology

Silica nanoparticles conjugated to PEI by covalent bonding were used in this set of tests. For a typical transfection process, HEK-293T cells were seeded in 6-well plates at a density of 2×10⁵ cells per well, and incubated for 24 h to achieve 70-90% confluency. 80 μg of PEI modified UQ silica particles was mixed with 2.5 μg of eGFP-pcDNA (loading of 31 μg pcDNA/mg silica) in 50 μL of PBS at 4° C. for 4 h. Note that this is a relatively low pcDNA loading level (significantly below the 100 μg/mg level measured for the Ram-SNP particles above) but was chosen so that the same loading was used across the different particle types, some of which are not capable of higher loadings, as shown in FIG. 50.

The mixture was then transferred into 2 mL of DMEM culture medium containing 10% FBS and 1% PS. The culture medium in the plates was then replaced by the particle containing medium, and then further cultured for 48 h. Subsequently, the cells were washed with PBS and then fixed with 500 μL of 4% PFA. The cells were viewed using confocal microscopy (LSM Zeiss 710) or collected for flow cytometry analysis (accuri M6).

Results

As shown above, silica nanoparticles covalently bound to PEI showed high pcDNA loading capacity and strong binding affinity. Here, the transfection efficiency is further investigated in the HEK-293T cell line. Confocal microscopy images clearly showed GFP expression in HEK-293T cells using different types of silica nanoparticles. Compared to silica nanoparticles modified with 1.8 k PEI, vectors modified with larger molecular weights of PEI showed improved delivery efficiency of pcDNA with brighter green fluorescence. However, it has been well documented that 25 k PEI exhibits severe cell toxicity. Thus modification using 10 k PEI is considered optimal. Comparing the three types of silica nanoparticles, the Ram-SNPs showed significantly enhanced pcDNA delivery efficiency with obvious and strong green fluorescence. This result clearly demonstrates that the unique structure of the Ram-SNPs provides superior transfection efficiency compared to similar silica particles that do not possess the unique spiky surface of the Ram-SNPs. The significance of this comparison is accentuated by the fact that the Ram-SNPs are disadvantaged versus the other SNPs by the stronger pcDNA binding affinity observed for the former, which likely leads to incomplete release of the pcDNA in the cell cytoplasm.

The pcDNA transfection efficiency was further quantitatively analysed using flow cytometry. As summarised in Table 23, the transfection efficiency of naked pcDNA is negligible at 0.8%, while Ram-SNPs modified with 10 k PEI showed the highest transfection efficiency of more than 27%, higher than the other silica particles that do not possess the same spiky silica surface and which showed efficiencies 4.4% and 9.6% for the S-SNPs and Ras-SNPs respectively. Comparison of the transfection efficiency of Ram-SNPs with PEI modified surfaces using 1.8 k, 10 k and 25 k molecular weight PEI shows the 10 k PEI variant to have the highest transfection efficiency with the efficiency of the 1.8 k and 25 k variants dropping away to 19.7% and 22.8% respectively.

The commercial product Lipofectamine 2000 showed much higher transfection efficacy of 98.8% relative to the non-optimised Ram-SNPs, as expected. The Lipofectamine formulation used the optimal pcDNA loading recommended by the manufacturer.

TABLE 23

| | Transfection efficiency (%) |
|---|---|
| None (naked pcDNA) | 0.8 |
| Ram-SNP (PEI 10k) | 27.2 |
| Ram-SNP (PEI 1.8k) | 19.7 |
| Ram-SNP (PEI 25k) | 22.8 |
| S-SNP (PEI 10k) | 4.4 |
| Ras-SNP (PEI 10k) | 9.6 |
| Lipofectamine 2000 | 98.8 |

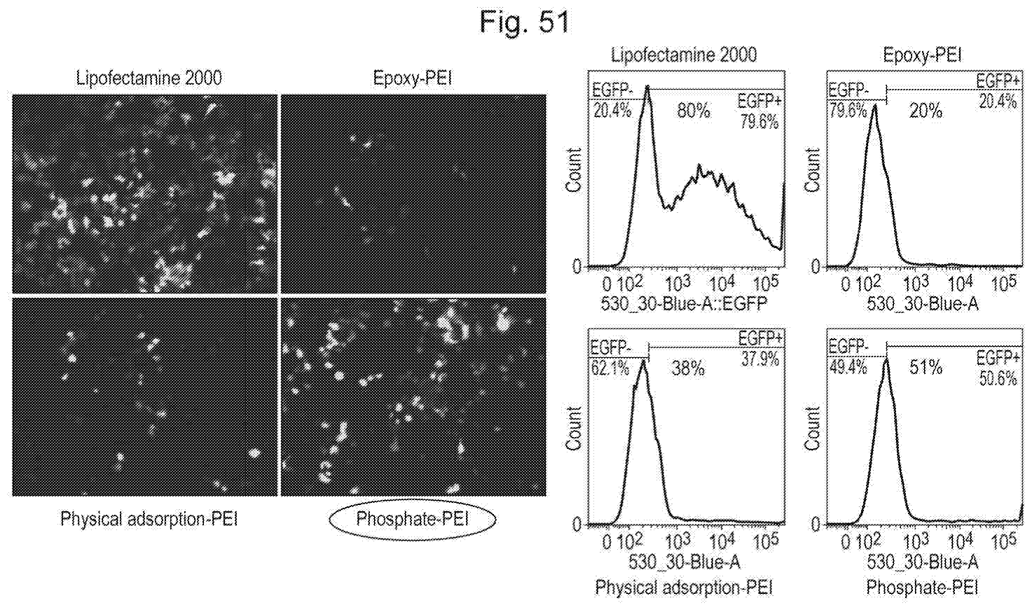

To further improve the transfection efficiency of the Ram-SNPs described above that rely on covalently-bound PEI surface modification, other modes of PEI conjugation were investigated for the Ram-SNPs. Here, Ram-SNPs were modified with 10 k PEI using phosphonate groups bound to the silica surface to act as a linker with the PEI, enabling strong electrostatic attraction with the PEI. Ram-SNPs with physically adsorbed PEI were also investigated. These nanoparticles were loaded with same dosage of pcDNA (31 μg/mg) for transfection in HEK-293T cells. Fluorescent microscopy and flow cytometry were used to analyse the transfection efficiency. As shown in FIG. 51, Lipofectamine 2000 showed strong green fluorescence with more than 80% of cells successfully transfected. The transfection efficiency of both epoxy-PEI modification and physical PEI adsorption were quite limited, with less than 40% of cells transfected. The phosphonate-PEI modification showed significantly improved transfection efficiency as demonstrated in fluorescent microscopy, with more than 51% of cell successfully transfected. Therefore, phosphonate-PEI modification is regarded as the optimal PEI modification mode.

Dose (silica dose) dependent transfection behaviour of Ram-SNPs modified with phosphonate-10 k PEI was also studied. By increasing the silica dosage from 40 μg (silica)/mL to 60 μg/mL and 80 Kg/mL the transfection efficiency increased from 53% to 77% and 89% respectively. The mass of pcDNA used in these experiments was kept constant such that the 80 μg/mL formulation had twice the number of particles and half the pcDNA loading (in terms of μg/mg) as the 40 μg/mL formulation. At the dosage of 80 μg/mL, the transfection efficiency of Ram-SNPs is similar to the commercial product Lipofectamine 2000 (90%). However, to be noted, the cellular toxicity of Ram-SNPs at a dosage of 80 μg/mL is quite high, giving some indication of the likely maximum dosage of silica particles that may be used in practical formulations. It is likely that in developing a commercial formulation, a compromise will have to be reached between transfection efficiency and cytotoxicity. Increasing the loading of pcDNA on the Ram-SNP particles may offer an attractive means of avoiding this trade-off however, as using higher pcDNA loadings would essentially mean less silica is required to be used, and likely lower cytotoxicity.

During the transfection experiment, pcDNA was first loaded onto PEI modified Ram-SNPs and typically 4 h is allowed for pcDNA loading. Investigating the loading process, it was found that more than 90% of the pcDNA is loaded onto the PEI modified Ram-SNPs within the first 5 minutes. This result agrees with the previous observation of strong binding affinity between the pcDNA and Ram-SNPs. After mixing pcDNA and PEI modified Ram-SNPs for 4 h and 5 min, their transfection efficiency was also studied via flow cytometry. Mixing pcDNA and particles for only 5 min results in transfection efficiency of 43.6% which is lower than the efficiency of 53.4% measured following the 4 h loading process.

Nucleic Acid Protection

Objective: Measure the capability of the Ram-SNPs to protect pcDNA from enzymatic degradation and compare performance with the commercial Lipofectamine 2000 product.

Methodology

0.5 μg of pcDNA was mixed with 15 μg of PEI modified Ram-SNPs (phosphonate group), then incubated at 4° C. for 2 h to achieve strong pcDNA and particle binding. The same amount of pcDNA was incubated with 1 μL of Lipofectamine 2000 at room temperature for 5 min. For DNase I digestion of pcDNA in the particles or Lipofectamine, 1 μL of 2 U/μL DNase I was added into the mixture and incubated at 37° C. for 30 min. To terminate the degradation, 1 μL of 500 mM EDTA was added into the mixture and then incubated at 65° C. for 10 min. To further identify the pcDNA residual after DNase I treatment, 1 μL of 40 mg/mL heparin PBS solution was added into the mixture and incubated at 37° C. for 1 h. The pcDNA-transfection agent complexes were analysed by gel electrophoresis. To identify the active pcDNA residual, the pcDNA-transfection agent formulations after DNase I treatment were transferred for transfection efficiency measurement in HEK-293T cells.

Results

To demonstrate the pcDNA protection capability of Ram-SNPs against DNase I, eGFP-pcDNA was loaded onto PEI modified Ram-SNPs then incubated with DNase I solution for 30 min. Electrophoresis results showed that naked pcDNA is easily degraded after DNase I treatment. The pcDNA loaded in the Ram-SNPs are strongly bound to particles without any free pcDNA released. After DNase I treatment, no pcDNA degradation band can be identified. After heparin treatment for pcDNA replacement, no released pcDNA band can be identified in the gel however an obvious band signal emerged in the well, which may result from the strong binding affinity between the pcDNA and Ram-SNPs. Due to the absence of pcDNA fragments, these data suggest that the Ram-SNP particles provide good protection of the pcDNA from nuclease degradation however the strong binding between the Ram-SNPs and the pcDNA prevent the direct visualisation of the intact pcDNA.

For the commercial transfection product Lipofectamine 2000, pcDNA was found to be easily released from the formulation, showing weak binding affinity between pcDNA and Lipofectamine. After DNase I treatment, the loosely bound pcDNA is easily degraded by the enzyme, showing no survival of this loosely bound pcDNA. After heparin replacement, a small amount of protected pcDNA was shown to be released from the Lipofectamine.

To further demonstrate there still remains active pcDNA in Ram-SNPs after DNase I treatment, pcDNA/Ram-SNP particles and pcDNA-Lipofectamine formulations before and after DNase treatment were used in in vitro transfection comparison in HEK-293T cells. Fluorescent microscopy images showed that the transfection efficiency of Lipofectamine 2000 decreased dramatically after DNase I treatment due to the severe degradation of pcDNA. However, the transfection efficiency of the Ram-SNPs remained relatively unchanged before and after DNase I treatment, further demonstrating the successful protection of pcDNA by the Ram-SNPs against enzyme digestion. This result indicates that the Ram-SNPs appear to provide a transfection efficiency advantage over the Lipofectamine agent due to the significant reduction in performance experienced by Lipofectamine in the presence of degradative enzymes.

In-Vitro Transfection Efficiency

Objective: compare the transfection efficiency of the Ram-SNPs with different particle sizes with that of the commercial Lipofectamine and in vivo JET products and elucidate cellular uptake mechanisms.

Compare Transfection Efficiency of Ram-SNPs with Different Particle Sizes, Free pcDNA, Lipofectamine and In-Vivo JET

Methodology—Synthesis Ram-SNPs with Different Particle Size

In the typical RF-silica synthesis, by changing the initial resorcinol and formaldehyde amount from 0.2 g/0.28 mL to 0.1 g/0.14 mL or 0.3 g/0.4 2 mL, the polymer core size is changed accordingly, finally resulting in smaller or larger Ram-SNP particle sizes. To be noted, by decreasing the resorcinol and formaldehyde amount, a longer polymerization time of 8 h is needed before TEOS addition. By increasing the resorcinol and formaldehyde amount, the time before TEOS addition can be shortened to 5 h. After the RF core polymerization, TEOS and second RF addition is followed by the typical synthesis process. The final silica nanoparticles were harvested and modified with phosphonate groups and conjugated with 10 k PEI for further transfection studies.

Results

Ram-SNPs used in the above studies had a particle size of approximately 330 nm, however the particle size may also influence the pcDNA transfection efficiency. Here, by varying the polymer core size in the RF-silica synthesis, Ram-SNPs with smaller diameters (approx. 180 nm) and larger diameters (approx. 500 nm) were fabricated. TEM images of these three Ram-SNP variants all exhibit spiky surface topography.

PEI modified Ram-SNPs with different particle size were used for eGFP-pcDNA transfection in HEK-293T cells at a silica dosage of 40 μg/mL. In comparison, commercially available transfection agents, Lipofetamine 2000 from Invitrogen and In-vivo JET from Polyplus were used according to the manufacturer's recommended protocol. Fluorescent microscopy and flow cytometry were used to analyse the transfection efficiency. Lipofectamine and especially in-vivo JET showed intense green fluorescence, with more than 90% of cells successfully transfected. The Ram-SNPs showed lower fluorescent intensity as expected for the low silica dosage of 40 μg/mL. Most importantly, a clear trend is seen in the increase in the transfection efficiency provided by the Ram-SNPs from 43% to 63% as the particle size is reduced from 500 nm to 180 nm.

Explore Cellular Uptake and Intracellular Trafficking Using Inhibitors of Specific Endocytosis Pathways

Methodology

Ram-SNPs with particle size of 180 nm were used here. After PEI modification, rhodamine isothiocyanate (RITC) was further conjugated to the particles by stirring PEI modified particles in 2 mg/mL RITC ethanol solution for 4 h. The RITC labelled particles were thoroughly washed by ethanol until no red colour could be identified in the supernatant. RITC labelled particles were then loaded with pcDNA for further uptake analysis. Prior to addition of particles, various internalization-inhibiting conditions were achieved via 1 h incubation at 37° C. in the medium. 100 μL of 1 μg/mL sucrose was added to 2 mL of medium (5% w/v) to inhibit clathrin-mediated endocytosis. Dynasore was added into the medium achieving a final concentration of 80 μM to inhibit dynamin dependent endocytosis. Low temperature treatment of cells (4° C.) was used for general endocytosis pathway analysis. pcDNA-nanoparticle formulations were then added to HEK-293T cells at 80-90% confluency and incubated for 4 h at 4 or 37° C. as required. Cells were harvested after 4 h of incubation and analysed via flow cytometry. Each group of experiments was conducted in triplicate.

Results

To identify the specific endocytosis pathways of Ram-SNPs into HEK-293T cells, different type of inhibitors were employed for cell treatment prior to particle addition. Ram-SNPs were stained with RITC exhibiting red fluorescence and flow cytometry was used to analyse the particle uptake with and without inhibitor treatment. There is no significant uptake inhibition after adding sucrose as an inhibitor, indicating the endocytosis pathway is not clathrin-mediated. However, HEK-293T cells by low temperature treatment and Dynasore addition showed significantly decreased particle uptake, indicating the Ram-SNPs are taken up by general and dynamin dependent endocytosis pathways.

pcDNA and Ram-SNP Binding Affinity

Methodology

15 μg of PEI modified Ram-SNPs (180 nm) were incubated with 0.5 μg of pcDNA for 2 h, then the mixture was further incubated with heparin with final concentration ranging from 0.5 to 10 mg/mL at 37° C. for 2 h. Then the mixture was further analysed by gel electrophoresis to identify the binding affinity of pcDNA and Ram-SNP particles.

Results

To further identify the binding affinity of pcDNA and Ram-SNPs, heparin competition assay was studied in a dose dependent manner. It was observed that pcDNA can be replaced from pcDNA-Ram-SNP particles at high concentrations of heparin. To be noted, at the heparin concentration of 0.5 mg/mL, the released pcDNA binding intensity is much lower than the ones treated at higher heparin concentration. This indicates there exists a strong binding affinity between pcDNA and Ram-SNP particles.

1. A process for producing a plurality of hollow inorganic nanoparticles, which process comprises:
(a) contacting a first monomer and a second monomer in a solvent to produce a composition comprising the solvent and a plurality of polymer nanoparticles;
(b) adding an inorganic compound precursor to the composition comprising the solvent and the plurality of polymer nanoparticles to produce a composition comprising the solvent and a plurality of inorganic compound-coated polymer nanoparticles;
(c) adding an additional amount of the first and second monomers to the composition comprising the solvent and the plurality of inorganic compound-coated polymer nanoparticles to produce a composition comprising the solvent and a plurality of composite nanoparticles; and
(d) heating the plurality of composite nanoparticles to produce the plurality of hollow inorganic nanoparticles,
wherein in step (a) the first monomer and the second monomer are contacted in the solvent at a temperature of at least 30° C.

2. A process according to claim 1, wherein the first monomer and the second monomer are contacted in the solvent at a temperature of from 30° C. to 70° C.

3. A process according to claim 1, wherein the first monomer and the second monomer are contacted in the solvent at a temperature of at least 30° C. for no more than four hours.

4. A process according to claim 1, wherein the inorganic compound is silica and the hollow inorganic nanoparticles are hollow silica nanoparticles.

5. A process according to claim 1, wherein step (a) comprises mixing the first monomer and the second monomer in the solvent,
which solvent preferably comprises water, an alcohol and ammonia.

6-13. (canceled)

14. A process according to claim 1, which process further comprises a step of cooling the composition comprising the solvent and the plurality of polymer nanoparticles in between step (a) and step (h),
wherein the composition comprising the solvent and the plurality of polymer nanoparticles is cooled at an average rate of from 0.5° C./min to 1.0° C./min for a time of from 10 minutes to 60 minutes.

15. (canceled)

16. A process according to claim 1, wherein following addition of an additional amount of the first and second monomers to the composition comprising the solvent and the plurality of inorganic compound-coated polymer nanoparticles, the concentration of first monomer is from 2.0 mM to 0.2 M and the concentration of the second monomer is from 2.0 mM to 0.2 M in the composition comprising the solvent, the plurality of inorganic compound-coated polymer nanoparticles and the first and second monomers.

17-20. (canceled)

21. A process according to claim 1, wherein each of the hollow inorganic nanoparticles comprises:
a shell comprising an inorganic compound;
a volume within the shell which does not comprise the inorganic compound; and
disposed on the exterior of the shell, a plurality of protrusions comprising the inorganic compound.

22. (canceled)

23. A process according to claim 1, which process further comprises:
(e) treating the plurality of hollow inorganic nanoparticles with an agent to produce a plurality of hollow inorganic nanoparticles loaded with the agent.

24-26. (canceled)

27. A process according to claim 23, which process comprises a step of treating the plurality of hollow inorganic nanoparticles with a phosphonate linker prior to treating the plurality of hollow inorganic nanoparti cies with the agent.

28-31. (canceled)

32. A plurality of hollow inorganic nanoparticles, wherein each of the hollow inorganic nanoparticles comprises:
a shell comprising an inorganic compound;
a volume within the shell which does not comprise the inorganic compound; and
disposed on the exterior of the shell, a plurality of protrusions comprising the inorganic compound; and
where the particle size of the plurality of hollow inorganic nanoparticles is from 100 to 500 nm.

33. A plurality of hollow inorganic nanoparticles, Wherein each of the hollow inorganic nanoparticles comprises:
a shell comprising an inorganic compound;
a volume within the shell which does not comprise the inorganic compound; and
disposed on the exterior of the shell, a plurality of protrusions comprising the inorganic compound; and
wherein the hollow inorganic nanoparticles further comprise a plurality of acidic groups bound to the inorganic compound.

34. A plurality of hollow inorganic nanoparticles according to claim 33, wherein the acidic groups are selected from phosphonate groups, phosphate groups, carboxylate groups and an alpha-keto carboxylate groups.

35-41. (canceled)

42. A composition comprising a plurality of hollow inorganic nanoparticles according to claim 32 and an agent.

43-44. (canceled)

45. A composition according to claim 42, wherein the agent is a pesticide, a herbicide, a therapeutic agent, a vaccine, a charge modifying agent, a transfection reagent, a nucleic acid, or a dye.

46. A composition according to claim 42, wherein the agent is polyethyleneimine.

47. A composition according to claim 42, wherein the plurality of hollow inorganic nanoparticles are functionalised with a phosphonate linker.

48-49. (canceled)

50. A composition as recited in claim 42, wherein the agent is an active agent, wherein the active agent is a vaccine and the composition is for use in the prevention or treatment of a disease in a subject by immunizing the subject against the disease using the vaccine.

51. A method of transfecting a nucleic acid into a cell, the method comprising treating the cell with a composition as defined in claim 42.

52-53. (canceled)

54. A composition as defined in claim 42 for use in a method of transfecting a nucleic acid into a cell, wherein the agent is a nucleic acid and the method comprises treating the cell with the composition comprising the plurality of hollow inorganic nanoparticles and the nucleic acid, and thereby transfecting the cell with the nucleic acid and stimulating an immune response.

55. (canceled)
